(19) United States
(12) Patent Application Publication
(10) Pub. No.: US 2024/0131189 A1
(43) Pub. Date: Apr. 25, 2024

(54) VARIANT ADENO-ASSOCIATED VIRUS (AAV) CAPSID POLYPEPTIDES AND GENE THERAPEUTICS THEREOF FOR TREATMENT OF HEARING LOSS

(71) Applicant: Eli Lilly and Company, Indianapolis, IN (US)

(72) Inventors: Steven Pennock, Leominster, MA (US); Adrian M. Timmers, Burlington, MA (US); Mark Shearman, Alachua, FL (US); Christopher Bartolome, Billerica, MA (US); Wang Xiaobo, San Diego, CA (US); Prahav Dinesh Mathur, San Diego, CA (US); Phillip M. Uribe, San Diego, CA (US); Stephanie Szobota, San Diego, CA (US); Bonnie E. Jacques, San Diego, CA (US); Alan C. Foster, San Diego, CA (US); Fabrice Piu, San Diego, CA (US)

(21) Appl. No.: 18/143,902

(22) Filed: May 4, 2023

Related U.S. Application Data
(63) Continuation of application No. PCT/US2021/058255, filed on Nov. 5, 2021.
(60) Provisional application No. 63/146,269, filed on Feb. 5, 2021.
(60) Provisional application No. 63/110,697, filed on Nov. 6, 2020.

(51) Int. Cl. A61K 48/00; A61K 38/17; A61P 27/16

(52) U.S. Cl. CPC A61K 48/0058; A61K 38/17; A61K 48/0066; A61P 27/16

(57) ABSTRACT

Described herein are variant adeno-associated virus (AAV) capsid polypeptides and gene therapeutics thereof for use in the treatment or prevention of hearing loss.

### CROSS-REFERENCE TO RELATED APPLICATIONS

This application is a continuation of International Application PCT/US2021/058255 filed on Nov. 5, 2021, which claims benefit under 35 U.S.C. § 119(e) of U.S. Provisional Application No. 63/110,697, filed Nov. 6, 2020, and U.S. Provisional Application No. 63/146,269, filed Feb. 5, 2021, the entire contents of each of which are incorporated herein by reference in their entirety.

### SEQUENCE LISTING

The instant application contains a Sequence Listing which has been submitted electronically in XML file format and is hereby incorporated by reference in its entirety. Said XML copy, created on Jan. 5, 2024, is named 138507-00203_SL.xml and is 96,575 bytes in size.

### TECHNICAL FIELD

Disclosed herein are variant adeno-associated virus (AAV) capsid polypeptides and gene therapeutics thereof for use in the treatment or prevention of hearing loss.

### SUMMARY

According to one aspect, the disclosure provides methods of treating or preventing hearing loss associated with deficiency of a gene, the method comprising administering to a subject in need thereof an effective amount of a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding the gene.

According to another aspect, the disclosure provides methods of delivering a nucleic acid sequence encoding a gene associated with hearing loss to an inner ear tissue or cell comprising administering to a subject in need thereof an effective amount of a recombinant adeno-associated virus (rAAV) virion comprising: (i) a variant AAV capsid polypeptide which exhibits increased tropism in inner ear tissues or cells as compared to a non-variant AAV capsid polypeptide; and (ii) a polynucleotide comprising a nucleic acid sequence encoding the gene.

According to some embodiments, the inner ear tissues or cells are cochlear tissues or cells, or vestibular tissues or cells. According to certain embodiments, the inner ear tissues or cells are cochlear tissues or cells.

According to some embodiments, the variant AAV capsid polypeptide is any variant AAV capsid polypeptide, optionally, selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; a variant AAV12 capsid polypeptide; and a variant Anc80 capsid polypeptide. According to certain embodiments, the variant AAV capsid polypeptide is a variant AAV2 capsid polypeptide.

According to some embodiments, the variant AAV capsid polypeptide comprises an amino acid sequence listed in Table 1, or an amino acid sequence having at least about 85%, 90%, 95%, or 99% sequence identity thereto, optionally, wherein the AAV capsid is selected from the group consisting of a VP1, VP2, or VP3 capsid polypeptide.

According to some embodiments, the variant AAV capsid polypeptide comprises an amino acid sequence listed in Table 1, or an amino acid sequence having at least about 85%, 90%, 95%, or 99% sequence homology thereto, optionally, wherein the AAV capsid is selected from the group consisting of a VP1, VP2, or VP3 capsid polypeptide.

According to some embodiments, the variant AAV capsid polypeptide comprises an amino acid sequence having one or more amino acid substitutions, insertions, and/or deletions relative to a wildtype AAV2 capsid polypeptide (SEQ ID NO: 18), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730. According to certain embodiments, the variant AAV capsid polypeptide comprises an amino acid sequence having one or more amino acid substitutions relative to a wildtype AAV2 capsid polypeptide (SEQ ID NO: 18) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.

According to some embodiments, the variant AAV capsid polypeptide comprises: (i) an amino acid sequence of any one of SEQ ID NOs: 27, 29, 31, 33, or 35; (ii) an amino acid sequence having at least about 85%, 90%, 95%, or 99% sequence identity to any one of SEQ ID NOs: 27, 29, 31, 33, or 35; or (iii) an amino acid sequence encoded by the nucleic acid sequence of any one of SEQ ID NOs: 26, 28, 30, 32, or 34. According to certain embodiments, the variant AAV capsid polypeptide comprises the amino acid sequence of SEQ ID NO: 27. According to certain embodiments, the variant AAV capsid polypeptide comprises the amino acid sequence of SEQ ID NO: 29. According to certain embodiments, the variant AAV capsid polypeptide comprises the amino acid sequence of SEQ ID NO: 31. According to certain embodiments, the variant AAV capsid polypeptide comprises the amino acid sequence of SEQ ID NO: 33. According to certain embodiments, the variant AAV capsid polypeptide comprises the amino acid sequence of SEQ ID NO: 35.

According to some embodiments, the variant AAV capsid polypeptide results in an increased level of rAAV tropism in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to a non-variant AAV capsid polypeptide.

According to some embodiments, the variant AAV capsid polypeptide results in an increased level of rAAV tropism in the inner ear tissues or cells, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a non-variant AAV capsid polypeptide.

According to some embodiments, the variant AAV capsid polypeptide results in an increased level of rAAV transduction efficiency in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to a non-variant AAV capsid polypeptide.

According to some embodiments, the variant AAV capsid polypeptide results in an increased level of rAAV transduction efficiency in the inner ear tissues or cells, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a non-variant AAV capsid polypeptide.

According to some embodiments, the method results in an increased expression of the gene in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to normal expression of the gene.

According to some embodiments, the method results in an increased expression of the gene in the inner ear tissues or cells, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to normal expression of the gene.

According to some embodiments, the method results in an overexpression of GJB2 (Connexin 26) expression in the inner ear tissues or cells.

According to some embodiments, the method results in a decreased level of rAAV neutralizing antibody (NAb) titers, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a control level.

According to some embodiments, the method results in a decreased level of inner ear inflammation or toxicity, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of inner ear inflammation or toxicity prior to administration. According to certain embodiments, the decreased level of inner ear inflammation or toxicity is as compared to a non-variant AAV capsid polypeptide. According to certain embodiments, the decreased level of inner ear inflammation or toxicity is as compared to that cause by a disease or disorder associated with hearing loss.

According to some embodiments, the method results in a delay in progression of inner ear inflammation or toxicity, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to progression of inner ear inflammation or toxicity prior to administration.

According to some embodiments, method results in a decreased level of hair cell loss, degeneration, and/or death, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of hair cell loss, degeneration, and/or death prior to administration.

According to some embodiments, the method results in a decreased level of spiral ganglion neuron loss, degeneration, and/or death, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of spiral ganglion neuron loss, degeneration, and/or death prior to administration.

According to some embodiments, the method results in a decreased auditory brainstem response (ABR) threshold at for example the 1 kHz frequency, 4 kHz frequency, 8 kHz frequency, and/or 16 kHz frequency, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of ABR threshold prior to administration.

According to some embodiments, the method results in an improved Distortion Product Otoacoustic Emissions (DPOAE) profile. According to certain embodiments, the method results in preventing, delaying or slowing down the deterioration of DPOAE profile.

According to some embodiments, the method results in an improved speech comprehension. According to certain embodiments, the method results in preventing, delaying or slowing down the deterioration of speech comprehension.

According to some embodiments, the control level is based on: a level obtained from the subject, optionally, a sample from the subject, prior to administration of the rAAV.

According to some embodiments, the control level is based on: a level resulting from the administration of a rAAV without the variant AAV capsid polypeptide, optionally, wherein the rAAV without the variant AAV capsid polypeptide comprises an rAAV capsid polypeptide selected from AAV2 and Anc80L65.

According to some embodiments, the method results in delivery to, and expression of a nucleic acid sequence encoding a gene associate with hearing loss, such as GJB2 in, a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, a border cell, an inner cochlear hair cell, an outer cochlear hair cell, a spiral ganglion neuron, a vestibular hair cell, a vestibular support cell, and/or a vestibular ganglion neuron.

According to some embodiments, the method results in delivery to, and expression of, a nucleic acid sequence encoding a gene associated with hearing loss, such as GJB2, in at least about 5%, 10%, 15%, 20%, 25%, 30%, 35%, 40%, 45%, 50%, 55%, 60%, 65%, 70%, 75%, 80%, 85%, 90%, 95%, or 99% of cells of the lateral wall or spiral ligament, support cells of the organ of Corti, fibrocytes of the spiral ligament, Claudius cells, Boettcher cells, cells of the spiral prominence, vestibular supporting cells, Hensen's cells, Deiters' cells, pillar cells, inner phalangeal cells, outer phalangeal cells, border cells, inner and outer cochlear hair cells, spiral ganglion neurons, vestibular hair cells, vestibular support cells, and/or vestibular ganglion neurons.

According to some embodiments, the gene is GJB2.

According to some embodiments, the nucleic acid sequence encoding GJB2 is a non-naturally occurring sequence. According to some embodiments, the nucleic acid sequence encoding GJB2 encodes mammalian GJB2. According to some embodiments, the nucleic acid sequence encoding GJB2 encodes human, mouse, non-human primate, or rat GJB2. According to some embodiments, the nucleic acid sequence encoding GJB2 comprises SEQ ID NO: 10. According to some embodiments, the nucleic acid sequence encoding GJB2 is codon optimized for mammalian expression. According to some embodiments, the nucleic acid sequence encoding GJB2 comprises SEQ ID NO: 11, SEQ ID NO: 12 or SEQ ID NO: 13. According to some embodiments, the nucleic acid sequence encoding GJB2 is codon optimized for expression in human, rat, non-human primate, guinea pig, mini pig, pig, cat, sheep, or mouse cells. According to some embodiments, the nucleic acid sequence encoding GJB2 is a cDNA sequence. According to some embodiments, the nucleic acid sequence encoding GJB2 further comprises an operably linked C-terminal tag or N-terminal tag. According to some embodiments, the tag is a FLAG-tag or a HA-tag.

According to some embodiments, the nucleic acid sequence encoding GJB2 is operably linked to a promoter. According to some embodiments, the promoter is an ubiquitously-active CBA, small CBA (smCBA), EF1a, CASI promoter, a cochlear-support cell promoter, GJB2 expression-specific GFAP promoter, small GJB2 promoter, medium GJB2 promoter, large GJB2 promoter, a sequential combination of 2-3 individual GJB2 expression-specific promoters, or a synthetic promoter. According to some embodiments, the promoter is optimized to drive sufficient GJB2 expression to treat or prevent hearing loss.

According to some embodiments, the nucleic acid sequence encoding GJB2 further comprises an operably linked 3′UTR regulatory region. According to some embodiments, the nucleic acid sequence encoding GJB2 further comprises an operably linked 3′UTR regulatory region comprising a Woodchuck Hepatitis Virus Postranscriptional Regulatory Element (WPRE).

According to some embodiments, the nucleic acid sequence encoding GJB2 further comprises an operably linked polyadenylation signal. According to some embodiments, the polyadenylation signal is an SV40 polyadenylation signal. According to some embodiments, the polyadenylation signal is a human growth hormone (hGH) polyadenylation signal.

According to some embodiments, the polynucleotide further comprises a 27-nucleotide hemagglutinin C-terminal tag or a 24-nucleotide Flag tag; operably linked to one of the following promoter elements optimized to drive high GJB2 expression: (a) an ubiquitously-active CBA, small CBA (smCBA), EF1a, or CASI promoter; (b) a cochlear-support cell or GJB2 expression-specific 1.68 kb GFAP, small/medium/large GJB2 promoters, a sequential combination of 2-3 individual GJB2 expression-specific promoters, or a synthetic promoter; operably linked to a 3′-UTR regulatory region comprising the Woodchuck Hepatitis Virus Posttranscriptional Regulatory Element (WPRE) followed by either a SV40 or human growth hormone (hGH) polyadenylation signal.

According to some embodiments, the polynucleotide further comprises an AAV genomic cassette, optionally, wherein: (i) the AAV genomic cassette is flanked by two sequence-modulated inverted terminal repeats, preferably about 143-bases in length; or (ii) the AAV genomic cassette is flanked by a self-complimentary AAV (scAAV) genomic cassette consisting of two inverted identical repeats, preferably no longer than 2.4 kb, separated by an about 113-bases scAAV-enabling ITR (ITRΔtrs) and flanked on either end by about 143-bases sequence-modulated ITRs.

According to some embodiments, the polynucleotide comprises a codon/sequence-optimized human GJB2 cDNA with or without a hemagglutinin C-terminal tag, preferably about 27-nucleotide in length, optionally about a 0.68 kilobase (kb) in size tag or a 24-nucleotide Flag tag; operably linked to one of the following promoter elements optimized to drive high GJB2 expression: (a) an ubiquitously-active CBA, preferably about 1.7 kb in size, small CBA (smCBA), preferably about 0.96 kb in size, EF1a, preferably about 0.81 kb in size, or CASI promoter, preferably about 1.06 kb in size; (b) a cochlear-support cell or GJB2 expression-specific GFAP promoter, preferably about 1.68 kb in size, small GJB2 promoter, preferably about 0.13 kb in size, medium GJB2 promoter, preferably about 0.54 kb in size, large GJB2 promoter, preferably about 1.0 kb in size, or a sequential combination of 2-3 individual GJB2 expression-specific promoters; operably linked to a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, and further comprising either two about 143-base sequence-modulated inverted terminal repeats (ITRs) flanking the AAV genomic cassette or a self-complimentary AAV (scAAV) genomic cassette consisting of two inverted identical repeats, preferably no longer than 2.4 kb, separated by an about 113-base scAAV-enabling ITR (ITRΔtrs) and flanked on either end by about 143-base sequence-modulated ITRs.

According to some embodiments, the hearing loss is genetic hearing loss. According to some embodiments, the hearing loss is DFNB1 hearing loss. According to some embodiments, the hearing loss is caused by a mutation in GJB2. According to some embodiments, the hearing loss is caused by an autosomal recessive GJB2 mutants (DFNB1). According to some embodiments, the hearing loss is caused by an autosomal dominant GJB2 mutants (DFNA3A).

According to some embodiments, the administration is to the cochlea or vestibular system, optionally, wherein the delivery comprises direct administration into the cochlea or vestibular system via the round window membrane (RWM), oval window, or semi-circular canals. According to some embodiments, the direct administration is injection. According to some embodiments, the administration is intravenous, intracerebroventricular, intracochlear, intrathecal, intramuscular, subcutaneous, or a combination thereof.

According to another aspect, the disclosure provides a composition for use in a method of treating or preventing hearing loss associated with deficiency of a gene comprising a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding the gene.

According to another aspect, the disclosure provides a composition for use in a method of delivering a nucleic acid sequence encoding a gene associated with hearing loss to an inner ear tissue or cell comprising administering to a subject in need thereof an effective amount of a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding gap junction protein beta 2 (GJB2).

According to some embodiments of the foregoing compositions, the inner ear tissues or cells are cochlear tissues or cells, or vestibular tissues or cells. According to some embodiments, the inner ear tissues or cells are cochlear tissues or cells.

According to some embodiments of the foregoing compositions, the variant AAV capsid polypeptide is selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; and a variant AAV12 capsid polypeptide. According to some embodiments, the variant AAV capsid polypeptide is a variant AAV2 capsid polypeptide. According to some embodiments, the variant AAV capsid polypeptide comprises an amino acid sequence listed in Table 1, or an amino acid sequence having at least about 85%, 90%, 95%, or 99% sequence identity thereto, optionally, wherein the AAV capsid is selected from the group consisting of a VP1, VP2, or VP3 capsid polypeptide. According to some embodiments, the variant AAV capsid polypeptide comprises an amino acid sequence listed in Table 1, or an amino acid sequence having at least about 85%, 90%, 95%, or 99% sequence homology thereto, optionally, wherein the AAV capsid is selected from the group consisting of a VP1, VP2, or VP3 capsid polypeptide. According to some embodiments, the variant AAV capsid polypeptide comprises an amino acid sequence having one or more amino acid substitutions, insertions, and/or deletions relative to a wildtype AAV2 capsid polypeptide (SEQ ID NO: 1), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730. According to some embodiments, the variant AAV capsid polypeptide comprises an amino acid sequence having one or more amino acid substitutions relative to a wildtype AAV2 capsid polypeptide (SEQ ID NO: 1) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F. According to some embodiments, the variant AAV capsid polypeptide comprises: (i) an amino acid sequence of any one of SEQ ID NOs: 27, 29, 31, 33, or 35; (ii) an amino acid sequence having at least about 85%, 90%, 95%, or 99% sequence identity to any one of SEQ ID NOs: 27, 29, 31, 33, or 35; or (iii) an amino acid sequence encoded by the nucleic acid sequence of any one of SEQ ID NOs: 26, 28, 30, 32, or 34. According to some embodiments, the variant AAV capsid polypeptide comprises the amino acid sequence of SEQ ID NO: 27. According to some embodiments, the variant AAV capsid polypeptide comprises the amino acid sequence of SEQ ID NO: 29. According to some embodiments, the variant AAV capsid polypeptide comprises the amino acid sequence of SEQ ID NO: 31. According to some embodiments, the variant AAV capsid polypeptide comprises the amino acid sequence of SEQ ID NO: 33. According to some embodiments, the variant AAV capsid polypeptide comprises the amino acid sequence of SEQ ID NO: 35.

According to some embodiments of the foregoing compositions, the gene is GJB2. According to some embodiments, the nucleic acid sequence encoding GJB2 is a non-naturally occurring sequence. According to some embodiments, the nucleic acid sequence encoding GJB2 encodes mammalian GJB2. According to some embodiments, the nucleic acid sequence encoding GJB2 encodes human, mouse, non-human primate, or rat GJB2. According to some embodiments, the nucleic acid sequence encoding GJB2 comprises SEQ ID NO: 10. According to some embodiments, the nucleic acid sequence encoding GJB2 is codon optimized for mammalian expression. According to some embodiments, the nucleic acid sequence encoding GJB2 comprises SEQ ID NO: 11, SEQ ID NO: 12 or SEQ ID NO: 13. According to some embodiments, the nucleic acid sequence encoding GJB2 is codon optimized for expression in human, rat, non-human primate, guinea pig, mini pig, pig, cat, sheep, or mouse cells. According to some embodiments, the nucleic acid sequence encoding GJB2 is a cDNA sequence. According to some embodiments of the foregoing compositions, the nucleic acid sequence encoding GJB2 further comprises an operably linked C-terminal tag or N-terminal tag. According to some embodiments, the tag is a FLAG-tag or a HA-tag.

According to some embodiments of the foregoing compositions, the nucleic acid sequence encoding GJB2 is operably linked to a promoter. According to some embodiments, the promoter is an ubiquitously-active CBA, small CBA (smCBA), EF1a, CASI promoter, a cochlear-support cell promoter, GJB2 expression-specific GFAP promoter, small GJB2 promoter, medium GJB2 promoter, large GJB2 promoter, a sequential combination of 2-3 individual GJB2 expression-specific promoters, or a synthetic promoter. According to some embodiments, the promoter is optimized to drive sufficient GJB2 expression to treat or prevent hearing loss.

According to some embodiments of the foregoing compositions, the nucleic acid sequence encoding GJB2 further comprises an operably linked 3′UTR regulatory region. According to some embodiments, the nucleic acid sequence encoding GJB2 further comprises an operably linked 3′UTR regulatory region comprising a Woodchuck Hepatitis Virus Postranscriptional Regulatory Element (WPRE).

According to some embodiments of the foregoing compositions, the nucleic acid sequence encoding GJB2 further comprises an operably linked polyadenylation signal. According to some embodiments, the polyadenylation signal is an SV40 polyadenylation signal. According to some embodiments, the polyadenylation signal is a human growth hormone (hGH) polyadenylation signal.

According to some embodiments of the foregoing compositions, the polynucleotide further comprising a 27-nucleotide hemagglutinin C-terminal tag or a 24-nucleotide Flag tag; operably linked to one of the following promoter elements optimized to drive high GJB2 expression: (a) an ubiquitously-active CBA, small CBA (smCBA), EF1a, or CASI promoter; (b) a cochlear-support cell or GJB2 expression-specific 1.68 kb GFAP, small/medium/large GJB2 promoters, a sequential combination of 2-3 individual GJB2 expression-specific promoters, or a synthetic promoter; operably linked to a 3′-UTR regulatory region comprising the Woodchuck Hepatitis Virus Posttranscriptional Regulatory Element (WPRE) followed by either a SV40 or human growth hormone (hGH) polyadenylation signal.

According to some embodiments of the foregoing compositions, the polynucleotide further comprises an AAV genomic cassette, optionally, wherein: (i) the AAV genomic cassette is flanked by two sequence-modulated inverted terminal repeats, preferably about 143-bases in length; or (ii) the AAV genomic cassette is flanked by a self-complimentary AAV (scAAV) genomic cassette consisting of two inverted identical repeats, preferably no longer than 2.4 kb, separated by an about 113-bases scAAV-enabling ITR (ITRΔtrs) and flanked on either end by about 143-bases sequence-modulated ITRs.

According to some embodiments of the foregoing compositions, the polynucleotide comprises a codon/sequence-optimized human GJB2 cDNA with or without a hemagglutinin C-terminal tag, preferably about 27-nucleotide in length, optionally about a 0.68 kilobase (kb) in size or a 24-nucleotide Flag tag; operably linked to one of the following promoter elements optimized to drive high GJB2 expression: (a) an ubiquitously-active CBA, preferably about 1.7 kb in size, small CBA (smCBA), preferably about 0.96 kb in size, EF1a, preferably about 0.81 kb in size, or CASI promoter, preferably about 1.06 kb in size; (b) a cochlear-support cell or GJB2 expression-specific GFAP promoter, preferably about 1.68 kb in size, small GJB2 promoter, preferably about 0.13 kb in size, medium GJB2 promoter, preferably about 0.54 kb in size, large GJB2 promoter, preferably about 1.0 kb in size, or a sequential combination of 2-3 individual GJB2 expression-specific promoters; operably linked to a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, and further comprising either two about 143-base sequence-modulated inverted terminal repeats (ITRs) flanking the AAV genomic cassette or a self-complimentary AAV (scAAV) genomic cassette consisting of two inverted identical repeats, preferably no longer than 2.4 kb, separated by an about 113-base scAAV-enabling ITR (ITRΔtrs) and flanked on either end by about 143-base sequence-modulated ITRs.

According to another aspect, the disclosure provides, a method of treating or preventing hearing loss comprising administering to a subject in need thereof an effective amount of a composition as described herein.

According to another aspect, the disclosure provides a method of delivering a nucleic acid sequence encoding a gene associated with hearing loss to an inner ear tissue or cell comprising administering to a subject in need thereof an effective amount of a composition as described herein.

According to another aspect, the disclosure provides a method of delivering a nucleic acid sequence encoding GJB2 to an inner ear tissue or cell comprising administering to a subject in need thereof an effective amount of a composition as described herein.

According to another aspect, the disclosure provides a kit comprising a composition as described herein and instructions for use.

### BRIEF DESCRIPTION OF THE DRAWINGS

FIG. 1A is a schematic of cochlear anatomy and cell types.

FIG. 1B shows a close up of the support cells. Shown are outer hair cells (01, 02, 03), inner hair cells (IHC), hensen's cells (h1, h2, h3, h4), deiters' cells (d1, d2, d3), pillar cells (p), inner phalangeal cells (IPC), outer phalangeal cells/border cells (bc).

FIG. 1C is a schematic of cochlear anatomy and cell types indicating regions of GJB2 expression.

FIG. 2 shows a schematic of GJB2 vector (genome) construct single stranded (ss)AAV-GJB2 and self-complementary scAAV-GJB2.

FIG. 3 shows the nucleic acid sequence of the CBA promoter (SEQ ID NO. 1).

FIG. 4 shows the nucleic acid sequence of the EF1a promoter (SEQ ID NO. 2).

FIG. 5 shows the nucleic acid sequence of the CASI promoter (SEQ ID NO. 3).

FIG. 6 shows the nucleic acid sequence of the smCBA promoter (SEQ ID NO. 4).

FIG. 7 shows the nucleic acid sequence of the GFAP promoter (SEQ ID NO. 5).

FIG. 8 shows the nucleic acid sequence of the GJB2 promoter (SEQ ID NO. 6). This promoter can have three different iterations: underlined sequence (128 bp), green shaded region (539 bp), and the entire sequence (1000 bp).

FIG. 9 shows the nucleic acid sequences of the following ITRs (AAV2) 5′-3′: for single stranded (ss) and self-complimentary (sc) AAV genomes (SEQ ID NO. 7); 3′-5′: for single stranded (ss) AAV genomes only (SEQ ID NO. 8); 3′-5′: for self-complimentary (sc) AAV genomes only (SEQ ID NO. 9).

FIG. 10 shows the nucleic acid sequence of the human wild-type GJB2 (hGJB2 wt) (SEQ ID NO. 10).

FIG. 11 shows the nucleic acid sequence of the human codon optimized GJB2 (hGJB2co3) (SEQ ID NO. 11).

FIG. 12 shows the nucleic acid sequence of the human codon optimized GJB2 (hGJB2co6) (SEQ ID NO. 12).

FIG. 13 shows the nucleic acid sequence of the human codon optimized GJB2 (hGJB2co9) (SEQ ID NO. 13).

FIG. 14 shows the nucleic acid sequence of an HA tag (SEQ ID NO. 14).

FIG. 15 shows the nucleic acid sequence of a Woodchuck Hepatitis Virus Posttranscriptional Regulatory Element (WPRE) (SEQ ID NO. 15).

FIG. 16 shows the nucleic acid sequence of a SV40 poly(A) terminator sequence (SEQ ID NO. 16).

FIG. 17 shows the nucleic acid sequence of a bGH poly(A) terminator sequence (SEQ ID NO. 17).

FIG. 18 shows the nucleic acid sequence of a FLAG tag (SEQ ID NO. 40).

FIG. 19 shows a bar graph comparing OMY-903, OMY-907, OMY-911, OMY-912, OMY-913, OMY-914, OMY-915, OMY-916, OMY-917, AAV2, and Anc80 GFP coverage normalized to OMY-906 (gray bar) in the spiral limbus.

FIG. 20 shows a bar graph comparing OMY-903, OMY-907, OMY-911, OMY-912, OMY-913, OMY-914, OMY-915, OMY-916, OMY-917, AAV2, and Anc80 GFP coverage normalized to OMY-906 (gray bar) in the organ of Corti.

FIG. 21 shows a bar graph comparing OMY-903, OMY-907, OMY-911, OMY-912, OMY-913, OMY-914, OMY-915, OMY-916, OMY-917, AAV2, and Anc80 GFP coverage normalized to OMY-906 (gray bar) in the spiral ligament.

FIG. 22 shows representative Z-stack images of GFP reporter expression in the middle region of the cochlea, including in the spiral ligament, organ of Corti, and spiral limbus, after treatment with OMY-903, Anc80, OMY-912, and wildtype AAV2.

FIG. 23 shows fluorescent images comparing OMY-912 GFP coverage at two doses: 2e9 vg and 2e10 vg.

FIG. 24 shows fluorescent images comparing OMY-915 GFP coverage at two doses: 2e9 vg and 2e10 vg.

FIG. 25 shows a bar graph comparing OMY-912 and OMY-915 GFP coverage in the spiral limbus at two doses: 2e9 vg and 2e10 vg.

FIG. 26 shows a bar graph comparing OMY-912 and OMY-915 GFP coverage in the organ of Corti at two doses: 2e9 vg and 2e10 vg.

FIG. 27 shows fluorescent images of FLAG antibody staining in support cells of rat cochlear explants indicating AAV-induced Connexin 26 expression. FLAG staining clearly overlapped with areas of connexin 26 expression demonstrating that the FLAG labeled protein is targeted to normal sites of connexin 26 expression. The FLAG antibody is shown in green, connexin 26 in magenta and nuclear staining by DAPI in blue. The left panel shows all colors merged, the middle panel shows just connexin 26 staining, and the right panel shows FLAG staining.

FIG. 28 shows fluorescent images of FLAG antibody staining in support cells of the mouse cochlea 2-6 weeks following intracochlear injection of AAV-GJB2-Flag indicating AAV-induced Connexin 26 expression. Representative images of the basal (base), middle (mid) and apical (apex) turns of the cochlear are shown. FLAG antibody is shown in red and nuclear staining by DAPI is shown in blue.

FIG. 29 shows fluorescent images of FLAG antibody staining in support cells of the adult mouse cochlea following intracochlear injection of AAV-GJB2-Flag indicating AAV-induced Connexin 26 expression. Representative images of the basal (base), middle (mid) and apical (apex) turns of the cochlear are shown. FLAG antibody is shown in red and nuclear staining by DAPI is shown in blue.

FIG. 30 shows images of non-human primate (NHP) cochlear sections evaluated by immunohistochemistry 12 weeks after intracochlear injection of OMY-913. DAB staining for GFP expression has been pseudo-colored red. FIG. 30 (Top panel) shows a low magnification image of the entire cochlea and demonstrates that consistent expression can be observed from base to apex throughout the cochlea after a single OMY-913 injection administered near the base via round window membrane injection. FIG. 30 (Bottom panel) shows that OMY-913 expression is observed in the regions relevant to GJB2 rescue, including the lateral wall (LW), organ of Corti (OC) support cells, and spiral limbus (SL).

FIG. 31 shows a line graph with hearing thresholds as measured by auditory brain stem response (ABR) across different frequencies in wild-type (WT) mice expressing Cx26 and inducible cre mice with Cx26 knockout (KO) measured at postnatal day 30 and postnatal day 60.

FIG. 32 shows a line graph with hearing thresholds as measured by auditory brain stem response (ABR) across different frequencies in wild-type (WT) mice expressing Cx26 and constitutive cre mice with Cx26 knockout (KO) measured at postnatal day 30.

FIG. 33 shows images of Cx26 expression in inducible cre mice with Cx26 knockout (KO) treated with vehicle or THERAPEUTIC-A.

FIG. 34 shows a timeline (top panel) of photobleaching and image capture for each FRAP trial, and a graph (bottom panel) showing that THERAPEUTIC A and THERAPEUTIC A-FLAG recover fluorescence faster than untransduced HeLa cells signifying that the transgene driven protein is likely forming functional gap junctions.

FIGS. 35A-D show a series of images showing that THERAPEUTIC A-FLAG expression is present at high levels throughout the length of the cochlea and forms membranous, plaque-like structures in the inner sulcus (FIG. 35A), Claudius cells (FIG. 35B), and other support cell types (FIG. 35C).

FIG. 36 is a series of images showing intracochlear injection of THERAPEUTIC A or THERAPEUTIC A-FLAG via the round window membrane with fenestration in the posterior semicircular canal in adult C57BL/6J mice at P30 age was safe and did not cause damage to the inner or outer hair cells at 42 days post surgery.

FIG. 37 is a series of images showing CX26-FLAG transduction (green) in the inner sulcus, Claudius cells and lateral wall fibrocytes cells at 14 days post surgery after THERAPEUTIC A-FLAG administration in adult (P30) C57BL/6J mice.

FIG. 38 is a series of images showing CX26-FLAG transduction (green) in the inner sulcus, Claudius cells and lateral wall fibrocytes cells at 14 days post-surgery after THERAPEUTIC A-FLAG administration in adult (P30) C57BL/6J mice.

FIG. 39A-B show an inducible mouse model (Rosa-cre) of GJB2 congenital hearing loss.

FIG. 39C shows that intracochlear injection of THERAPEUTIC A-FLAG into wildtype mice during the postnatal period provides extensive cochlear coverage including all cell types that natively express CX26. The intracochlear injection was made at the age when rescue studies were performed in the Rosa-cre animal model.

FIG. 39D-E show that THERAPEUTIC A demonstrated substantial rescue of ABR thresholds across multiple frequencies, restoration of CX26 expression and preservation of cochlear morphology in the Rosa-cre animal model.

FIG. 40A-B show a mouse model with inner ear deletion of GJB2 by crossing Cx26^{loxp/loxp} mice with mice expressing Cre driven by the inner ear specific promoter P0 (P0-Cre).

FIG. 40C shows that intracochlear injection of THERAPEUTIC A-FLAG into wildtype mice during the postnatal period provides extensive cochlear coverage including all cell types that natively express CX26. The intracochlear injection was made at the age when rescue studies were performed in the P0-cre animal model.

FIG. 40D-E show that THERAPEUTIC A demonstrated substantial rescue of ABR thresholds across multiple frequencies, restoration of CX26 expression and preservation of cochlear morphology in the P0-cre animal model.

FIG. 41 shows that intracochlear injection of THERAPEUTIC A-FLAG exhibits a high degree of transduction and good tropism in non-human primate (NHP).

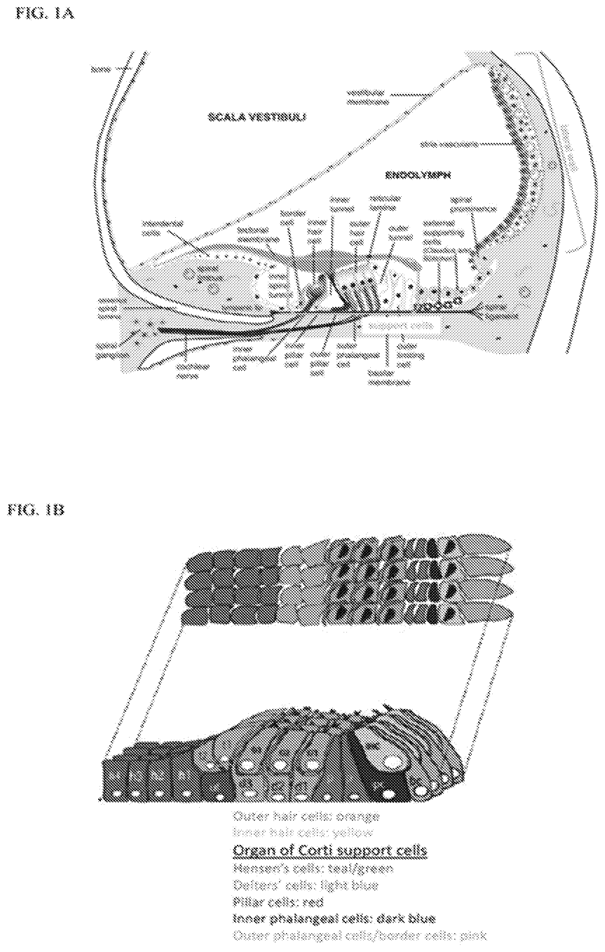

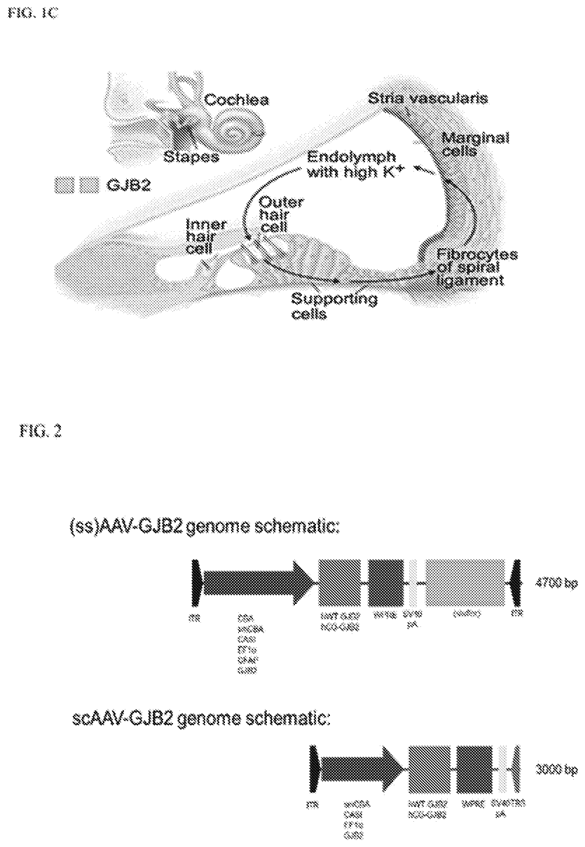

### DETAILED DESCRIPTION

Nonsyndromic hearing loss and deafness (DFNB1; also known as Connexin 26 deafness) is autosomal recessive and is characterized by congenital non-progressive mild-to-profound sensorineural hearing impairment. The GJB2 gene encodes connexin-26 which is expressed in cochlear support cells, forming gap junctions that are involved in intercellular communication that is important for cochlea homeostasis, including the control of potassium gradients which play a significant role in the survival and function of hair cells and normal hearing. Mutations in GJB2 impair gap junctions and cochlear homeostasis leading to hair cell dysfunction and hearing loss.

According to the NIDCD, 2 to 3 out of every 1,000 children in the United States are born with some degree of hearing loss, with more than half due to genetic factors. Mutations in the GJB2 gene which encodes the gap junction protein Connexin 26 (CX26) are the most common forms of hereditary deafness, responsible for >50% of cases across various ethnic groups. While in most subjects the onset of hearing loss is prelingual and moderate to severe, in some subjects, hearing loss due to loss of CX26 is mild and progressive. In the inner ear, expression of CX26 is vital for the function of various non-sensory cell types including support cells and fibrocytes.

Genetic testing can be used to diagnose DFNB1 by identifying biallelic pathogenic variants in GJB2 which encompass sequence variants and variants in upstream cis-regulatory elements that alter expression of the gap junction beta-2 protein (Connexin 26). When the GJB2 pathogenic variants causing DFNB1 are detected in an affected family member, carrier testing for at-risk relatives, prenatal testing for pregnancies at increased risk, and preimplantation genetic diagnosis are possible. Smith & Jones. Nonsyndromic Hearing Loss and Deafness, DFNB1. 1998. In: Adam, et al. Eds. GeneReviews. University of Washington, Seattle; Kemperman et al. Journal of the Royal Society of Medicine 2002 95: 171-177. The present disclosure recognizes that the cochlea is surgically accessible and local application into a relatively immune-protected environment is possible, and that gene therapy using viral vectors is useful for treating hearing loss. The present disclosure also recognizes that gene therapies capable of increased tropism and transduction in inner ear tissues and cells can effectively treat hearing loss associated with deficiency of a gene.

The disclosure relates to variant adeno-associated virus (AAV) capsid polypeptides which exhibit increased tropism in inner ear tissues or cells, e.g., as compared to non-variant AAV capsid polypeptides. The variant AAV capsid polypeptides described herein can be incorporated into an rAAV vector and/or a rAAV virion through which a gene can be packaged for targeted delivery to patients suffering from hearing loss associated with deficiency of the gene, including patients with autosomal mutations, recessive or dominant, in the gene. In particular embodiments, the gene is gap junction protein beta 2 (GJB2). Mutations in GJB2 impair gap junctions and cochlear homeostasis, leading to disruption of cochlear structure, hair cell dysfunction and hearing loss. A goal of GJB2 gene therapy as described herein is to restore functional gap junctions and preserve hair cells to improve hearing.

### Definitions

Unless defined otherwise, all technical and scientific terms used herein have the meaning commonly understood by a person skilled in the art. The following references provide one of skill with a general definition of many of the terms used in this disclosure. Singleton et al. Dictionary of Microbiology and Molecular Biology (2^{nd} Ed. 1994); The Cambridge Dictionary of Science and Technology (Walker ed., 1988); The Glossary of Genetics, 5th Ed., R. Rieger et al. (Eds.), Springer Verlag (1991); and Hale & Marham, The Harper Collins Dictionary of Biology (1991). As used herein, the following terms have the meanings ascribed to them below, unless specified otherwise.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

As used herein, the terms “administer,” “administering,” “administration,” and the like, are meant to refer to methods that are used to enable delivery of therapeutics or pharmaceutical compositions to the desired site of biological action.

As used herein, the term “AAV” is an abbreviation for adeno-associated virus, and may be used to refer to the virus itself or derivatives thereof, e.g., AAV vectors, AAV virus particles, and/or AAV virions. The term covers all subtypes and both naturally occurring and recombinant forms, except where required otherwise. In some embodiments, an AAV may be referred to by its capsid polypeptide, e.g., by its variant capsid polypeptide. For example, an AAV comprising an OMY-913 variant capsid polypeptide may be referred to herein as “OMY-913”.

As used herein, the term “AAV virion” or “AAV virus” or “AAV viral particle” or “AAV vector particle” is meant to refer broadly to a complete virus particle, such as for example a wild type AAV virion particle, which comprises single stranded genome DNA packaged into AAV capsid proteins. The single stranded nucleic acid molecule is either sense strand or antisense strand, as both strands are equally infectious. The term “rAAV viral particle” refers to a recombinant AAV virus particle, i.e., a particle that is infectious but replication defective. A rAAV viral particle comprises single stranded genome DNA packaged into AAV capsid proteins. In certain embodiments, the AAV capsid protein is a variant AAV capsid protein which exhibits increased tropism in inner ear tissues or cells, e.g., as compared to a non-variant AAV capsid protein. The amino acids sequences and nucleotide sequences of exemplary variant AAV capsid proteins are provided in Table 1.

As used herein, the term “bioreactor” is meant to refer broadly to any apparatus that can be used for the purpose of culturing cells.

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

As used herein, the term “gene of interest (GOI),” as used herein refers broadly to a heterologous sequence introduced into an AAV expression vector, and typically refers to a nucleic acid sequence encoding a protein of therapeutic use in humans or animals. In some embodiments, the GOI is a gene associated with hearing loss. In some embodiments, the gene is gap junction protein beta 2 (GJB2). Other genes associated with hearing loss (e.g., hearing loss associated with deficiency of a gene) that may be used according to the methods described herein are known in the art and described, e.g., in Shearer et al., “Hereditary Hearing Loss and Deafness Overview”, 2017, incorporated by reference in its entirety herein. Examples of genes associated with hearing loss (e.g., hearing loss associated with deficiency of a gene) include, but are not limited to, ACTG1, ADCY1, ADGRV1, AIFM1, BDP1, BSND, BTD, CABP2, CCDC50, CD164, CDC14A, CDH23, CEACAM16, CIB2, CLDN14, CLIC5, CLRN1, COCH, COL11A1, COL11A2, COL2A1, COL4A3, COL4A4, COL4A5, COL4A6, COL9A1, COL9A2, COL9A3, DCDC2, DIAPHI, DMXL2, DSPP, EDN3, EDNRB, ELMOD3, EPS8, EPS8L2, ESPN, ESRRB, EYA1, EYA4, FAM189A2, GIPC3, GJB2, GJB3, GJB6, GPSM2, GRHL2, GRXCR1, GRXCR2, GSDME, HARS1, HGF, HOMER2, ILDR1, KARS1, KCNE1, KCNQ1, KCNQ4, LHFPL5, LOXHD1, LRTOMT, MARVELD2, MCM2, MET, MIR96, MITF, MSRB3, MT-CO1, MT-RNR1, MT-TS1, MYH14, MYH9, MYO15A, MYO1A, MYO3A, MYO6, MYO7A, MYO7A, NARS2, NF2, OSBPL2, OTOA, OTOF, OTOG, OTOGL, P2RX2, PAX3, PCDH15, PEX7, PHYH, PJVK, PNPT1, POU3F4, POU4F3, PRPS1, PTPRQ, RDX, RIPOR2, ROR1, S1PR2, SERPINB6, SIX1, SIX5, SLC17A8, SLC22A4, SLC26A4, SLC26A5, SMPX, SOX10, STRC, SYNE4, TBC1D24, TECTA, TIMM8A, TJP2, TMC1, TMEM132E, TMIE, TMPRSS3, TPRN, TRIOBP, TSPEAR, USH1C, USH1G, USH2A, WBP2, WFS1, and WHRN.

As used herein, the term “hearing loss” is meant to refer to a diminished sensitivity to the sounds normally heard by a subject. The severity of a hearing loss is categorized according to the increase in volume above the usual level necessary before the listener can detect it. According to some embodiments, hearing loss may be characterized by increases in the threshold volume at which an individual perceives tones at different frequencies. In certain embodiments, hearing can be measured in decibels (dB). In certain embodiments, the threshold or 0 dB mark for each frequency refers to the level at which a normal subject, e.g., a normal human subject, perceives a tone burst 50% of the time. In certain embodiments, hearing is considered normal if a subject's thresholds are within 15 dB of normal thresholds. In certain embodiments, severity of hearing loss is graded as follows: mild is 26-40 dB, moderate is 41-55 dB, moderately severe is 56-70 dB, severe is 71-90 dB, and profound is 90 dB. In certain embodiments, the methods described herein can reduce and/or slow the progression of hearing loss in a subject from one level, e.g., mild, to another level, e.g., moderate, moderately severe, severe, and/or profound. In certain embodiments, the methods described herein can improve and/or reverse the progression of hearing loss in a subject from one level, e.g., moderate, moderately severe, severe, and/or profound, to another level, e.g., mild. In certain embodiments, hearing loss is associated with deficiency of a gene. In certain embodiments, hearing loss is associated with deficiency of a gene selected from the group consisting of ACTG1, ADCY1, ADGRV1, AIFM1, BDP1, BSND, BTD, CABP2, CCDC50, CD164, CDC14A, CDH23, CEACAM16, CIB2, CLDN14, CLIC5, CLRN1, COCH, COL11A1, COL11A2, COL2A1, COL4A3, COL4A4, COL4A5, COL4A6, COL9A1, COL9A2, COL9A3, DCDC2, DIAPHI, DMXL2, DSPP, EDN3, EDNRB, ELMOD3, EPS8, EPS8L2, ESPN, ESRRB, EYA1, EYA4, FAM189A2, GIPC3, GJB2, GJB3, GJB6, GPSM2, GRHL2, GRXCR1, GRXCR2, GSDME, HARS1, HGF, HOMER2, ILDR1, KARS1, KCNE1, KCNQ1, KCNQ4, LHFPL5, LOXHD1, LRTOMT, MARVELD2, MCM2, MET, MIR96, MITF, MSRB3, MT-CO1, MT-RNR1, MT-TS1, MYH14, MYH9, MYO15A, MYO1A, MYO3A, MYO6, MYO7A, MYO7A, NARS2, NF2, OSBPL2, OTOA, OTOF, OTOG, OTOGL, P2RX2, PAX3, PCDH15, PEX7, PHYH, PJVK, PNPT1, POU3F4, POU4F3, PRPS1, PTPRQ, RDX, RIPOR2, ROR1, S1PR2, SERPINB6, SIX1, SIX5, SLC17A8, SLC22A4, SLC26A4, SLC26A5, SMPX, SOX10, STRC, SYNE4, TBC1D24, TECTA, TIMM8A, TJP2, TMC1, TMEM132E, TMIE, TMPRSS3, TPRN, TRIOBP, TSPEAR, USH1C, USH1G, USH2A, WBP2, WFS1, and WHRN. In certain embodiments, the hearing loss is associated with a mutation, such as a substitution, a deletion, a insertion, and/or a duplication, in a gene described herein. In certain embodiments, the hearing loss is associated with two or more mutations (e.g., 2, 3, 4, 5, 6, 7, 8, 9, or 10 or more mutations) in a gene described herein. In some embodiments, the two or more mutations can occur in the same gene or different genes. In some embodiments, the two or more mutations can occur in the same allele or different alleles of a gene. In some embodiments, the hearing loss may be associated with a heterozygous mutation in a gene described herein. In some embodiments, the hearing loss may be associated with a homozygous mutation in a gene described herein. In certain embodiments, the hearing loss is syndromic, which involves other presenting abnormalities along with hearing impairment. In certain embodiments, the hearing loss is nonsyndromic, which occur when there are no other problems associated with an individual other than hearing loss. In certain embodiments, dominant and recessive hearing loss results from the allelic mutation in some genes, syndromic and non-syndromic hearing loss is caused by mutations in the same gene, and recessive hearing loss may be caused by a combination of two mutations in different genes from the same functional group. In certain embodiments, the hearing loss is hereditary. In certain embodiments, the hearing loss is autosomal dominant nonsyndromic hearing impairment. In certain embodiments, the hearing loss is autosomal recessive nonsyndromic hearing impairment. In certain embodiments, the hearing loss is X-linked nonsyndromic hearing impairment. In certain embodiments, the hearing loss is mitochondrial syndromic hearing impairment. In certain embodiments, the hearing loss is acquired hearing loss. In certain embodiments, the hearing loss is progressive hearing loss. In certain embodiments, hearing loss in a subject may be associated with an underlying disease or disorder, for example, Waardenburg syndrome (WS), a branchiootorenal spectrum disorder, neurofibromatosis 2 (NF2), Stickler syndrome, Usher syndrome type I, Usher syndrome type II, Usher syndrome type III, Pendred syndrome, Jervell and Lange-Nielsen syndrome, biotinidase deficiency, Refsum disease, Alport syndrome, and/or deafness-dystonia-optic neuronopathy syndrome (Mohr-Tranebjaerg syndrome).

As used herein, the terms “herpesvirus” or “herpesviridae family, are meant to refer broadly to the general family of enveloped, double-stranded DNA viruses with relatively large genomes. The family replicates in the nucleus of a wide range of vertebrate and invertebrate hosts, in preferred embodiments, mammalian hosts, for example in humans, horses, cattle, mice, and pigs. Exemplary members of the herpesviridae family include cytomegalovirus (CMV), herpes simplex virus types 1 and 2 (HSV1 and HSV2) and varicella zoster (VZV) and Epstein Barr Virus (EBV).

As used herein, the term “heterologous,” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

As used herein, the term “infection,” is meant to refer broadly to delivery of heterologous DNA into a cell by a virus. The term “co-infection” as used herein means “simultaneous infection,” “double infection,” “multiple infection,” or “serial infection” with two or more viruses. Infection of a producer cell with two (or more) viruses will be referred to as “co-infection.” The term “transfection” refers to a process of delivering heterologous DNA to a cell by physical or chemical methods, such as plasmid DNA, which is transferred into the cell by means of electroporation, calcium phosphate precipitation, or other methods well known in the art.

As used herein, the term “inner ear cells” or “cells of the inner ear” refers to inner hair cells (IHCs) and outer hair cells (OHCs), spiral ganglion neurons, vestibular hair cells, vestibular ganglion neurons, supporting cells and cells in the stria vascularis, spiral ligament or spiral limbus. Supporting cells refer to cells in the ear that are not excitable, e.g., cells that are not hair cells or neurons. In some embodiments, the tissues and cells of the inner ear include cells of the lateral wall or spiral ligament, support cells of the organ of Corti, fibrocytes of the spiral ligament, Claudius cells, Boettcher cells, cells of the spiral prominence, vestibular supporting cells, Hensen's cells, Deiters' cells, pillar cells, inner phalangeal cells, outer phalangeal cells, and/or border cells.

As used herein, the term “inverted terminal repeat” or “ITR” sequence is meant to refer to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost nucleotides of the ITR can be present in either of two alternative orientations, leading to heterogeneity between different AAV genomes and between the two ends of a single AAV genome.

As used herein, the term “isolated” molecule (e.g., an isolated nucleic acid or protein or cell) means it has been identified and separated and/or recovered from a component of its natural environment.

As used herein, the term “middle ear” is meant to refer to the space between the tympanic membrane and the inner ear.

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts.

As used herein, the term “non-naturally occurring” is meant to refer broadly to a protein, nucleic acid, ribonucleic acid, or virus that does not occur in nature. For example, it may be a genetically modified variant, e.g., cDNA or codon-optimized nucleic acid.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the gene of interest or portion thereof (e.g., the GJB2 gene or portion thereof), or regulatory elements. A nucleic acid molecule can be single-stranded or double-stranded. A “GJB2 nucleic acid” refers to a nucleic acid that comprises the GJB2 gene or a portion thereof, or a functional variant of the GJB2 gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

As used herein, the terms “operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

Similarly, “sequence homology”, as used herein, also refers to a method of determining the relatedness of two sequences. To determine sequence homology, two or more sequences are optimally aligned as described above, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 95% of the amino acid residues or nucleotides in the reference sequence must match or comprise a conservative substitution with another amino acid or nucleotide, or a number of amino acids or nucleotides up to 5% of the total amino acid residues or nucleotides, not including conservative substitutions, in the reference sequence may be inserted into the reference sequence.

As used herein, the term “pharmaceutical composition” or “composition” is meant to refer to a composition or agent described herein (e.g. a recombinant adeno-associated (rAAV) expression vector and/or an rAAV virion), optionally mixed with at least one pharmaceutically acceptable chemical component, such as, though not limited to carriers, stabilizers, diluents, dispersing agents, suspending agents, thickening agents, excipients and the like.

As used herein, the terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. According to some embodiments, the promoter is highly specific for support cell expression in the cochlea. According to some embodiments, the promoter is an endogenous GJB2 promoter. According to some embodiments, the promoter is a synthetic promoter. According to some embodiments, the promoter is selected from the group consisting of a CBA promoter, smCBA promoter, a CASI promoter, a GFAP promoter, and an elongation factor-1 alpha (EF1a) promoter. A “chicken beta-actin (CBA) promoter” refers to a polynucleotide sequence derived from a chicken beta-actin gene (e.g., Gallus gallus beta actin, represented by GenBank Entrez Gene ID 396526). A “smCBA” promoter refers to the small version of the hybrid CMV-chicken beta-actin promoter. A “CASI” promoter refers to a promoter comprising a portion of the CMV enhancer, a portion of the chicken beta-actin promoter, and a portion of the UBC enhancer.

As used herein, the term “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “recombinant HSV,” “rHSV,” and “rHSV vector,” is meant to refer broadly to isolated, genetically modified forms of herpes simplex virus type 1 (HSV) containing heterologous genes incorporated into the viral genome. By the term “rHSV-rep2cap2” or “rHSV-rep2cap1” is meant an rHSV in which the AAV rep and cap genes from either AAV serotype 1 or 2 have been incorporated into the rHSV genome, in certain embodiments, a DNA sequence encoding a therapeutic gene of interest has been incorporated into the viral genome.

As used herein, a “subject” or “patient” or “individual” to be treated by the method of the present disclosure is meant to refer to either a human or non-human animal. According to some embodiments, the subject is a child. According to some embodiments, the subject is an infant. A “nonhuman animal” includes any vertebrate or invertebrate organism. In some embodiments, the subject is suffering from hearing loss associated with deficiency of a gene, such as the GJB2 gene.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In certain embodiments, introduction of a GJB2 transgene into a cell results in the formation of functional gap junctions.

As used herein, a “transgene expression cassette” or “expression cassette” comprises the gene sequences that a nucleic acid vector is to deliver to target cells. These sequences include the gene of interest (e.g., GJB2 nucleic acids or variants thereof), one or more promoters, and minimal regulatory elements.

As used herein, the terms “treatment” or “treating” a disease or disorder are meant to refer to alleviation of one or more signs or symptoms of the disease or disorder, diminishment of extent of disease or disorder, stabilized (e.g., not worsening) state of disease or disorder, preventing spread of disease or disorder, delay or slowing of disease or disorder progression, amelioration or palliation of the disease or disorder state, and remission (whether partial or total), whether detectable or undetectable. For example, a gene of interest, such as GJB2, when expressed in an effective amount (or dosage) is sufficient to prevent, correct, and/or normalize an abnormal physiological response, e.g., a therapeutic effect that is sufficient to reduce by at least about 30 percent, more preferably by at least 50 percent, most preferably by at least 90 percent, a clinically significant feature of disease or disorder. “Treatment” can also refer to prolonging survival as compared to expected survival if not receiving treatment.

As used herein, the term “vector” is meant to refer to a recombinant plasmid or virus that comprises a nucleic acid to be delivered into a host cell, either in vitro or in vivo.

As used herein, the term “recombinant viral vector” is meant to refer to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

As used herein, the term “recombinant AAV vector (rAAV vector)” is meant to refer to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”. In certain embodiments, the AAV virus capsid is a variant AAV capsid as described herein.

As used herein, the term a “rAAV virus” or “rAAV viral particle” or “rAAV viron” is meant to refer to a viral particle composed of at least one AAV capsid protein and an encapsidated rAAV vector genome. In certain embodiments, the AAV capsid protein is a variant AAV capsid protein, such as a variant AAV capsid protein which exhibits increased ropism in inner ear tissues or cells, e.g., as compared to a non-variant AAV capsid protein. The amino acids sequences and nucleotide sequences of exemplary variant AAV capsid proteins that may be used according to the methods described herein are provided in Table 1.

The term “variant” or “variants”, with regard to polypeptides, such as capsid polypeptides refers to a polypeptide sequence differing by at least one amino acid from a parent polypeptide sequence, also referred to as a non-variant polypeptide sequence. In some embodiments, the polypeptide is a capsid polypeptide and the variant differs by at least one amino acid substitution. Amino acids also include naturally occurring and non-naturally occurring amino acids as well as derivatives thereof. Amino acids also include both D and L forms.

The terms “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate. In certain embodiments, the variant AAV capsid polypeptides described herein exhibit increased tropism in inner ear tissues or cells e.g., as compared to a non-variant AAV capsid polypeptide. In certain embodiments, the variant AAV capsid polypeptides described herein exhibit increased transduction in inner ear tissues or cells, e.g., as compared to a non-variant AAV capsid polypeptide. In certain embodiments, the variant AAV capsid polypeptides described herein exhibit increased tropism and/or transduction in inner ear tissues or cells e.g., as compared to a non-variant AAV capsid polypeptide. In certain embodiments, the variant AAV capsid polypeptides that exhibit increased tropism and/or transduction in inner ear tissues or cells, e.g., as compared to a non-variant AAV capsid polypeptide, are provided in Table 1.

### Nucleic Acids

The present disclosure provides promoters, expression cassettes, vectors, kits, and methods that can be used in the treatment of hearing loss, e.g., hearing loss associated with deficiency of a gene. In some embodiments, the hearing loss is hereditary hearing impairment. Certain aspects of the disclosure relate to delivering a heterologous nucleic acid to tissues and cells of the inner ear of a subject comprising administering a recombinant adeno-associated virus (rAAV) vector and/or virion. According to some aspects, the disclosure provides methods of treating or preventing hearing loss, e.g., hearing loss associated with deficiency of a gene, comprising delivery of a composition comprising rAAV vectors and/or rAAV virions described herein to the subject, wherein the rAAV vector and/or rAAV virion comprises a heterologous nucleic acid (e.g. a nucleic acid encoding GJB2).

In certain embodiments, the rAAV vector comprises a heterologous nucleic acid encoding a gene associated with hearing loss. Examples of genes associated with hearing loss (e.g., hearing loss associated with deficiency of a gene) include, but are not limited to, ACTG1, ADCY1, ADGRV1, AIFM1, BDP1, BSND, BTD, CABP2, CCDC50, CD164, CDC14A, CDH23, CEACAM16, CIB2, CLDN14, CLIC5, CLRN1, COCH, COL11A1, COL11A2, COL2A1, COL4A3, COL4A4, COL4A5, COL4A6, COL9A1, COL9A2, COL9A3, DCDC2, DIAPHI, DMXL2, DSPP, EDN3, EDNRB, ELMOD3, EPS8, EPS8L2, ESPN, ESRRB, EYA1, EYA4, FAM189A2, GIPC3, GJB2, GJB3, GJB6, GPSM2, GRHL2, GRXCR1, GRXCR2, GSDME, HARS1, HGF, HOMER2, ILDR1, KARS1, KCNE1, KCNQ1, KCNQ4, LHFPL5, LOXHD1, LRTOMT, MARVELD2, MCM2, MET, MIR96, MITF, MSRB3, MT-CO1, MT-RNR1, MT-TS1, MYH14, MYH9, MYO15A, MYO1A, MYO3A, MYO6, MYO7A, MYO7A, NARS2, NF2, OSBPL2, OTOA, OTOF, OTOG, OTOGL, P2RX2, PAX3, PCDH15, PEX7, PHYH, PJVK, PNPT1, POU3F4, POU4F3, PRPS1, PTPRQ, RDX, RIPOR2, ROR1, S1PR2, SERPINB6, SIX1, SIX5, SLC17A8, SLC22A4, SLC26A4, SLC26A5, SMPX, SOX10, STRC, SYNE4, TBC1D24, TECTA, TIMM8A, TJP2, TMC1, TMEM132E, TMIE, TMPRSS3, TPRN, TRIOBP, TSPEAR, USH1C, USH1G, USH2A, WBP2, WFS1, and WHRN. In certain embodiments, the rAAV vector and/or rAAV virion comprises a heterologous nucleic acid encoding GJB2.

The gene most commonly mutated among subjects with hereditary hearing impairment (HI), GJB2, encodes the connexin-26 (Cx26) gap-junction channel protein that underlies both intercellular communication among supporting cells and homeostasis of the cochlear fluids, endolymph and perilymph. GJB2 lies at the DFNB1 locus on 13q12. GJB2 is 5513 bp long and contains two exons (193 bp and 2141 bp long, respectively) separated by a 3179-bp intron (Kiang et al., 1997). Transcription is initiated from a single start site and leads to the synthesis of a 2334-nucleotide mRNA (GenBank NM_004004.5), which is considered canonical.

According to some embodiments, the gene of interest (e.g., GJB2) is optimized to be superior in expression (and/or function) to the wildtype gene (e.g., wildtype GJB2), and further has the ability to discriminate (at the DNA/RNA level) from wildtype (e.g., wildtype GJB2).

FIG. 2 shows a schematic of an exemplary GJB2 vector (genome) construct single stranded (ss)AAV-GJB2 and self-complementary scAAV-GJB2.

The human wild-type GJB2 is an important element that codes for a major gap junction protein that is required for normal hearing. Loss of GJB2 causes massive cell death of various cell types in the inner ear following onset of hearing. A “GJB2 nucleic acid” refers to a nucleic acid that comprises the GJB2 gene or a portion thereof, or a functional variant of the GJB2 gene or a portion thereof. A functional variant of a gene includes a variant of the gene with minor variations such as, for example, silent mutations, single nucleotide polymorphisms, missense mutations, and other mutations or deletions that do not significantly alter gene function.

According to some embodiments, the disclosure provides a nucleic acid encoding a mammalian GJB2 protein. According to some embodiments, the disclosure provides a nucleic acid encoding a wild-type GJB2 protein. According to some embodiments, the disclosure provides a nucleic acid encoding a wild-type human, mouse, non-human primate, or rat GJB2 protein. According to some embodiments, the disclosure provides a nucleic acid encoding a human wild-type GJB2 protein. According to some embodiments, the nucleic acid sequence encoding the human wild-type GJB2 protein is 678 bp in length. According to one embodiment, the nucleic acid encoding the human wild-type GJB2 protein comprises SEQ ID NO: 10. According to one embodiment, the nucleic acid is at least 85% identical to SEQ ID NO: 10. According to one embodiment, the nucleic acid is at least 90% identical to SEQ ID NO: 10. According to one embodiment, the nucleic acid is at least 95% identical to SEQ ID NO: 10. According to one embodiment, the nucleic acid is at least 99% identical to SEQ ID NO: 10. According to one embodiment, the nucleic acid consists of SEQ ID NO: 10.

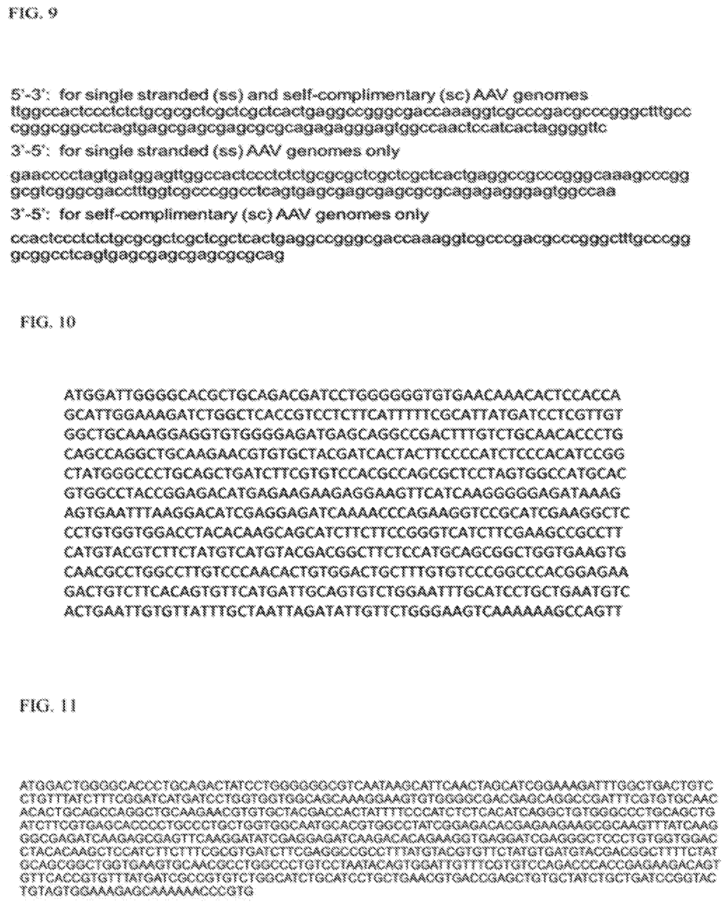

FIG. 10 shows the nucleic acid sequence of the human wild-type GJB2 (hGJB2 wt) (SEQ ID NO. 10).

According to some embodiments, the disclosure provides a nucleic acid encoding a GJB2 protein, wherein the nucleic acid sequence is codon optimized for mammalian expression. According to certain embodiments, the disclosure provides a nucleic acid encoding a GJB2 protein, wherein the nucleic acid sequence is codon optimized for expression in human, rat, non-human primate, guinea pig, mini pig, pig, cat, sheep, or mouse cells. The human codon optimized GJB2 is an important element that codes for a major gap junction protein that is required for normal hearing. Codon optimization is performed to enhance protein expression of GJB2.

According to some embodiments, the disclosure provides a nucleic acid encoding a GJB2 protein, wherein the nucleic acid sequence encoding the GJB2 protein is a non-naturally occurring sequence.

According to some embodiments, the disclosure provides a nucleic acid encoding a human codon optimized GJB2 protein.

According to some embodiments, the nucleic acid sequence encoding the human codon optimized GJB2 protein is 678 bp in length. According to one embodiment, the nucleic acid encoding the human codon optimized GJB2 protein comprises SEQ ID NO: 11. According to one embodiment, the nucleic acid is at least 85% identical to SEQ ID NO: 11. According to one embodiment, the nucleic acid is at least 90% identical to SEQ ID NO: 11. According to one embodiment, the nucleic acid is at least 95% identical to SEQ ID NO: 11. According to one embodiment, the nucleic acid is at least 99% identical to SEQ ID NO: 11. According to one embodiment, the nucleic acid consists of SEQ ID NO: 11.

FIG. 11 shows the nucleic acid sequence of the human codon optimized GJB2 (hGJB2co3) (SEQ ID NO. 11).

According to some embodiments, the nucleic acid sequence encoding the human codon optimized GJB2 protein is 678 bp in length. According to one embodiment, the nucleic acid encoding the human codon optimized GJB2 protein comprises SEQ ID NO: 12. According to one embodiment, the nucleic acid is at least 85% identical to SEQ ID NO: 12. According to one embodiment, the nucleic acid is at least 90% identical to SEQ ID NO: 12. According to one embodiment, the nucleic acid is at least 95% identical to SEQ ID NO: 12. According to one embodiment, the nucleic acid is at least 99% identical to SEQ ID NO: 12. According to one embodiment, the nucleic acid consists of SEQ ID NO: 12.

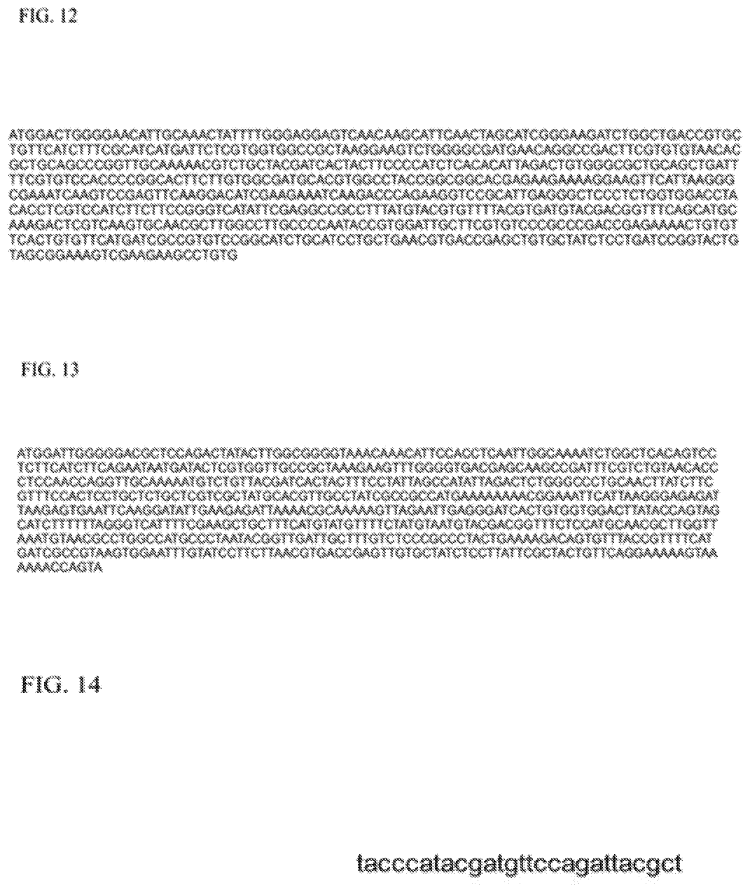

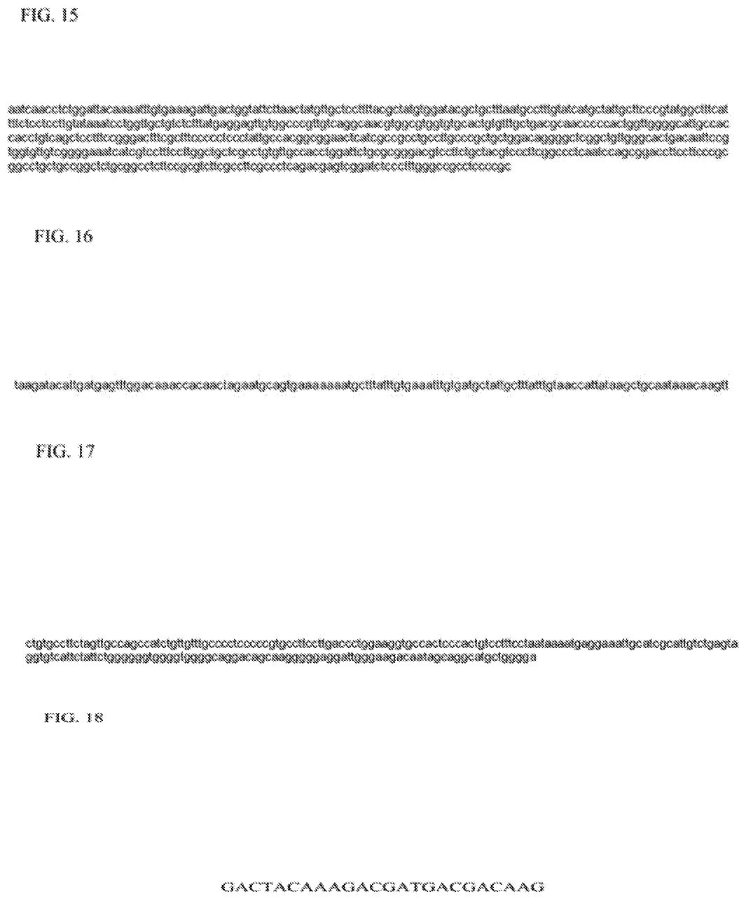

FIG. 12 shows the nucleic acid sequence of the human codon optimized GJB2 (hGJB2co6) (SEQ ID NO. 12).

According to some embodiments, the nucleic acid sequence encoding the human codon optimized GJB2 protein is 678 bp in length. According to one embodiment, the nucleic acid encoding the human codon optimized GJB2 protein comprises SEQ ID NO: 13. According to one embodiment, the nucleic acid is at least 85% identical to SEQ ID NO: 13. According to one embodiment, the nucleic acid is at least 90% identical to SEQ ID NO: 13. According to one embodiment, the nucleic acid is at least 95% identical to SEQ ID NO: 13. According to one embodiment, the nucleic acid is at least 99% identical to SEQ ID NO: 13. According to one embodiment, the nucleic acid consists of SEQ ID NO: 13.

FIG. 13 shows the nucleic acid sequence of the human codon optimized GJB2 (hGJB2co9) (SEQ ID NO. 13).

In certain embodiments, the rAAV vector comprises a nucleic acid sequence encoding a variant AAV capsid polypeptide. According to some embodiments, the rAAV vector comprises a nucleic acid sequence encoding a variant AAV capsid polypeptide that exhibits increased tropism in inner ear tissues or cells, e.g., as compared to a non-variant AAV capsid polypeptide.

According to some embodiments, the rAAV vector comprises a nucleic acid sequence encoding a variant AAV capsid polypeptide selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; and a variant AAV12 capsid polypeptide. According to some embodiments, the rAAV vector comprises a nucleic acid sequence encoding a a variant AAV2 capsid polypeptide.

According to some embodiments, the rAAV vector comprises a nucleic acid sequence encoding a variant AAV capsid polypeptide listed in Table 1, or a nucleic acid sequence having at least about 85%, 90%, 95%, or 99% sequence identity thereto.

According to some embodiments, the rAAV vector comprises a nucleic acid sequence encoding a variant AAV capsid polypeptide which comprises an amino acid sequence listed in Table 1, or an amino acid sequence having at least about 85%, 90%, 95%, or 99% sequence identity thereto.

According to some embodiments, the rAAV vector comprises a nucleic acid sequence encoding an AAV capsid selected from the group consisting of a VP1, VP2, or VP3 capsid polypeptide.

According to some embodiments, the rAAV vector comprises a nucleic acid sequence encoding a variant AAV capsid polypeptide comprising an amino acid sequence having one or more amino acid substitutions, insertions, and/or deletions relative to a wildtype AAV2 capsid polypeptide (SEQ ID NO: 18), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730.

According to some embodiments, the rAAV vector comprises a nucleic acid sequence encoding a variant AAV capsid polypeptide comprising an amino acid sequence having one or more amino acid substitutions relative to a wildtype AAV2 capsid polypeptide (SEQ ID NO: 18) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.

According to some embodiments, the rAAV vector comprises a nucleic acid sequence encoding a variant AAV capsid polypeptide comprising: (i) an amino acid sequence of any one of SEQ ID NOs: 27, 29, 31, 33, or 35; (ii) an amino acid sequence having at least about 85%, 90%, 95%, or 99% sequence identity to any one of SEQ ID NOs: 27, 29, 31, 33, or 35; or (iii) an amino acid sequence encoded by the nucleic acid sequence of any one of SEQ ID NOs: 26, 28, 30, 32, or 34. According to some embodiments, the rAAV vector comprises a nucleic acid sequence encoding, a variant AAV capsid polypeptide comprising the amino acid sequence of SEQ ID NO: 27. According to some embodiments, the rAAV vector comprises a nucleic acid sequence encoding, a variant AAV capsid polypeptide comprising the amino acid sequence of SEQ ID NO: 29. According to some embodiments, the rAAV vector comprises a nucleic acid sequence encoding, a variant AAV capsid polypeptide comprising the amino acid sequence of SEQ ID NO: 31. According to some embodiments, the rAAV vector comprises a nucleic acid sequence encoding, a variant AAV capsid polypeptide comprising the amino acid sequence of SEQ ID NO: 33. According to some embodiments, the rAAV vector comprises a nucleic acid sequence encoding, a variant AAV capsid polypeptide comprising the amino acid sequence of SEQ ID NO: 35.

### Promoters

Various promoters are contemplated for use in the present disclosure.

According to some embodiments, the promoter is an endogenous GJB2 promoter. The GJB2 promoter is a support-cell specific promoter and can transduce cells of the inner ear that express the GJB2 gene; this promoter can be used for production of scAAV given its short length. According to some embodiments, the promoter comprises SEQ ID NO: 6. According to some embodiments, the promoter consists of SEQ ID NO: 6. FIG. 8 shows the nucleic acid sequence of the GJB2 promoter (SEQ ID NO. 6).

According to some embodiments, the promoter is a CBA promoter. The CBA promoter is a strong ubiquitous promoter that can transduce multiple cell types in the inner ear. According to some embodiments, the promoter comprises SEQ ID NO: 1. According to some embodiments, the promoter consists of SEQ ID NO: 1. FIG. 3 shows the nucleic acid sequence of the CBA promoter (SEQ ID NO. 1).

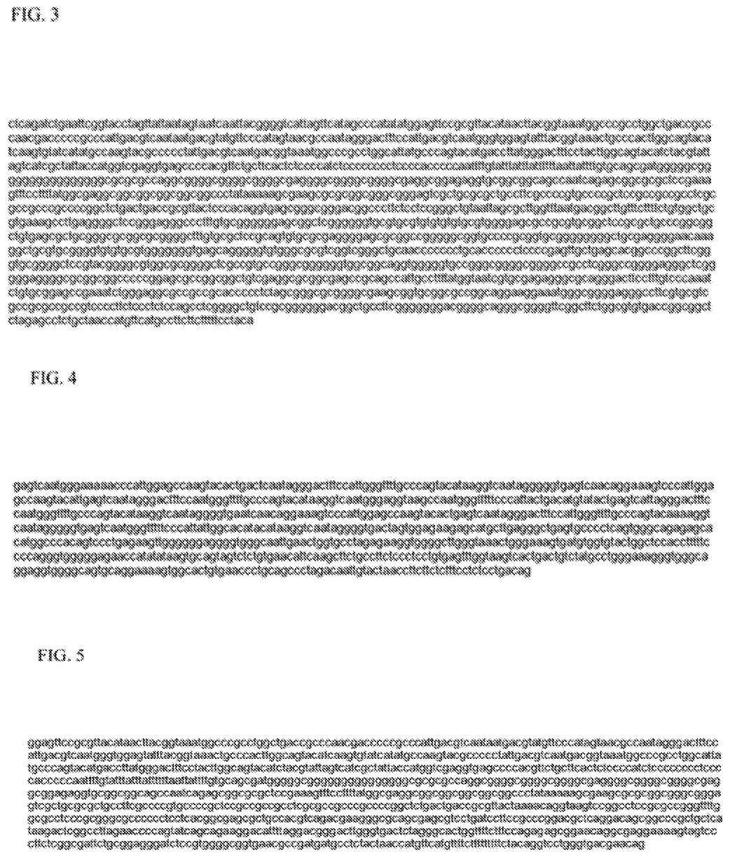

According to some embodiments, the promoter is an EF1a promoter. The EF1a promoter is a strong ubiquitous promoter of mammalian origin that can transduce multiple cell types in the inner ear, and can be used for production of scAAV given its short length. According to some embodiments, the promoter comprises SEQ ID NO: 2. According to some embodiments, the promoter consists of SEQ ID NO: 2. FIG. 4 shows the nucleic acid sequence of the EF1a promoter (SEQ ID NO. 2).

According to some embodiments, the promoter is a CASI promoter. The CASI promoter is a strong ubiquitous promoter that can transduce multiple cell types in the inner ear, and can be used for production of scAAV given its short length. According to some embodiments, the promoter comprises SEQ ID NO: 3. According to some embodiments, the promoter consists of SEQ ID NO: 3. FIG. 5 shows the nucleic acid sequence of the CASI promoter (SEQ ID NO. 3).

According to some embodiments, the promoter is a smCBA promoter. The smCBA promoter is a strong ubiquitous promoter that can transduce multiple cell types in the inner ear, and can be used for production of scAAV given its short length. According to some embodiments, the promoter comprises SEQ ID NO: 4. According to some embodiments, the promoter consists of SEQ ID NO: 4. FIG. 6 shows the nucleic acid sequence of the smCBA promoter (SEQ ID NO. 4.).

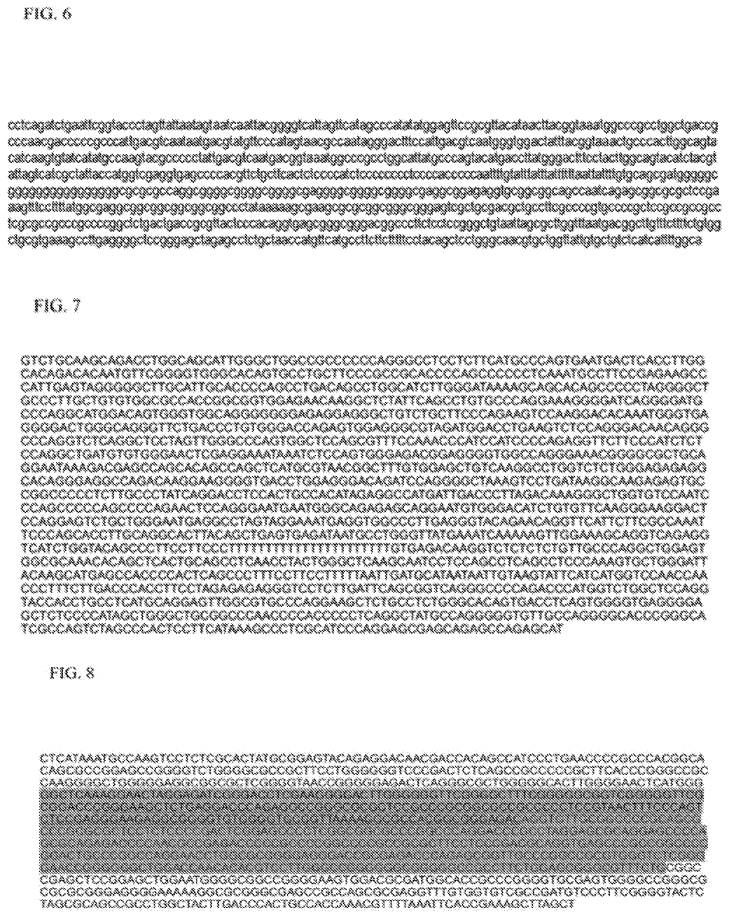

According to some embodiments, the promoter is a GFAP promoter. The GFAP promoter is cell-specific and has activity in support cells of the inner ear. According to some embodiments, the promoter comprises SEQ ID NO: 5. According to some embodiments, the promoter consists of SEQ ID NO: 5. FIG. 7 shows the nucleic acid sequence of the GFAP promoter (SEQ ID NO. 5).

According to some embodiments, the promoter is a synthetic promoter. In certain embodiments, a synthetic promoter is a sequence of DNA that does not exist in nature and which has been designed to control gene expression of a target gene, e.g., GJB2.

### Inverted Terminal Repeats

The inverted terminal repeat (ITR) sequences are required for efficient multiplication of the AAV genome, due to their ability to form hairpin structures that allows synthesis of the second DNA strand. scAAV shortened ITRs (TRS) form an intra-molecular double-stranded DNA template, thus removing the rate-limiting step of second-strand synthesis.

FIG. 9 shows the nucleic acid sequences of the following ITRs (AAV2) 5′-3′: for single stranded (ss) and self-complimentary (sc) AAV genomes (SEQ ID NO. 7); 3′-5′: for single stranded (ss) AAV genomes only (SEQ ID NO. 8); 3′-5′: for self-complimentary (sc) AAV genomes only (SEQ ID NO. 9).

### Gene Therapy for Hearing Loss

The disclosure generally provides methods for producing recombinant adeno-associated virus (AAV) viral particles comprising a gene construct (e.g., a GJB2 gene construct) and their use in methods of gene therapy for hearing loss, e.g., hearing loss associated with deficiency of a gene. The AAV vectors and AAV virions as described herein are particularly efficient at delivering nucleic acids (e.g., GJB2 gene construct) to inner ear tissues and cells. Methods to create, evaluate, and utilize recombinant adeno-associated virus (rAAV) therapeutic vectors capable of efficiently delivering a gene, such as GJB2, into cells for expression and subsequent secretion are described herein. Optimally-modified gene of interest (GOI) cDNA and associated genetic elements for use in recombinant adeno-associated virus (rAAV)-based gene therapy for hearing loss, e.g., hearing loss associated with deficiency of a gene, are described herein. More specifically, optimally-modified GJB2/Connexin26 (Cx26) cDNA and associated genetic elements for use in recombinant adeno-associated virus (rAAV)-based gene therapy for genetic hearing loss, including the treatment and/or prevention of DFNB1 and DFNA3A-associated congenital deafness, are described herein.

Recombinant adeno-associated virus (rAAV) vector can efficiently accommodate both target gene, e.g., GJB2 target gene, and associated genetic elements. Furthermore, such vectors can be designed to specifically express the gene, e.g., GJB2, in therapeutically relevant inner ear tissues and cells, such as the supporting cells of the cochlea. The disclosure describes a method to create, evaluate, and utilize rAAV therapeutic vectors and r AAV virions able to efficiently deliver the functional gene, e.g., GJB2 gene, to patients. of interest

In some embodiments, the GJB2 gene construct may comprise: (1) codon/sequence-optimized 0.68 kb human GJB2 cDNA with or without a 27-nucleotide hemagglutinin (HA)C-terminal tag; (2) one of the following promoter elements optimized to drive high GJB2 expression: (a) an ubiquitously-active 1.7 kb CBA, 0.96 kb small CBA (smCBA), 0.81 kb EF1a, or 1.06 kb CASI promoter; (b) a cochlear-support cell or GJB2 expression-specific 1.68 kb GFAP, 0.13/0.54/1.0 kb small/medium/large GJB2 promoters, or a sequential combination of 2-3 individual GJB2 expression-specific promoters, or a synthetic promoter; (3) a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, (4) either two 143-base sequence-modulated inverted terminal repeats (ITRs) flanking the AAV genomic cassette or a self-complimentary AAV (scAAV) genomic cassette consisting of two inverted identical repeats (each no longer than 3.0 kb) separated by a 113-base scAAV-enabling ITR (ITRΔtrs) and flanked on either end by 143-base sequence-modulated ITRs; and (5) a protein capsid variant optimally suited for cochlear delivery. In some embodiments, the nucleic acid sequence encoding GJB2 may comprise an operably linked C-terminal tag or N-terminal tag, such as a FLAG-tag or a HA-tag.

The HA tag is human influenza hemagglutinin, a surface glycoprotein used as a general epitope tag in expression vectors, facilitating detection of the protein of interest. The FLAG tag (peptide sequence DYKDDDDK (SEQ ID NO: 41)) is a short, hydrophilic protein tag commonly used as a general epitope tag in expression vectors, facilitating detection of the protein of interest. Woodchuck Hepatitis Virus Posttranscriptional Regulatory Element (WPRE) is a DNA sequence that enhances expression of the protein of interest by generating a tertiary structure that stabilizes its mRNA. According to certain embodiments, other regulatory sequences may be used. In certain embodiments, a regulatory sequences that may be used according to the present disclosure comprises a DNA sequence that when transcribed creates a tertiary structure that enhances expression of a target gene, such as GJB2. The poly(A) sequence is an important element that promotes RNA processing and transcript stability. The SV40 and bGH polyA sequences are terminator sequences that signals the end of a transcriptional unit. According to certain embodiments, other polyA terminator sequences may be used.

According to some embodiments, the AAV vectors and AAV virions described herein are particularly suited to deliver and express a gene, such as GJB2, in inner ear tissues or cells, including in the cochlear support cells. According to some embodiments, the AAV vectors and AAV virions described herein are particularly suited to deliver and express a gene, such as GJB2, in one or more of the external support cells and/or the organ of Corti support cells. According to some embodiments, the AAV vectors and AAV virions described herein are particularly suited to deliver and express a gene, such as GJB2, in one or more of the outer hair cells, the inner hair cells, hensen's cells, deiters' cells, pillar cells, inner phalangeal cells and/or outer phalangeal cells/border cells.

### Adeno-Associated Virus (AAV)

Adeno-Associated Virus (AAV) is a non-pathogenic single-stranded DNA parvovirus. AAV has a capsid diameter of about 20 nm. Each end of the single-stranded DNA genome contains an inverted terminal repeat (ITR), which is the only cis-acting element required for genome replication and packaging. The AAV genome carries two viral genes: rep and cap. The virus utilizes two promoters and alternative splicing to generate four proteins necessary for replication (Rep 78, Rep 68, Rep 52 and Rep 40). A third promoter generates the transcript for three structural viral capsid proteins, 1, 2 and 3 (VP1, VP2 and VP3), through a combination of alternate splicing and alternate translation start codons. Berns & Linden Bioessays 1995; 17:237-45. The three capsid proteins share the same C-terminal 533 amino acids, while VP2 and VP1 contain additional N-terminal sequences of 65 and 202 amino acids, respectively. The AAV virion contains a total of 60 copies of VP1, VP2, and VP3 at a 1:1:20 ratio, arranged in a T-1 icosahedral symmetry. Rose et al. J Virol. 1971; 8:766-70. AAV requires Adenovirus (Ad), Herpes Simplex Virus (HSV) or other viruses as a helper virus to complete its lytic life-cycle. Atchison et al. Science, 1965; 149:754-6; Hoggan et al. Proc Natl Acad Sci USA, 1966; 55:1467-74. In the absence of the helper virus, wild-type AAV establishes latency by integration with the assistance of Rep proteins through the interaction of the ITR with the chromosome. Berns & Linden (1995). In certain embodiments, the AAV described herein comprise variant AAV capsid polypeptides that exhibit increased tropism and/or transduction in inner ear tissues or cells, e.g., as compared to a non-variant AAV capsid polypeptide. Exemplary variant AAV capsid polypeptides are provided in Table 1.

### AAV Serotypes

There are a number of different AAV serotypes, including AAV1, AAV2, AAV3, AAV4, AAV5, AAV6, AAV7, AAV8, AAV9, AAV10, AAV11, AAV12, AAVrh8, AAVrh10, Anc80L65, and variants or hybrids thereof. In vivo studies have shown that the various AAV serotypes display different tissue or cell tropisms. For example, AAV1 and AAV6 are two serotypes that, are efficient for the transduction of skeletal muscle. Gao, et al. Proc Nat Acad Sci USA, 2002; 99:11854-11859; Xiao, et al. J Virol. 1999; 73:3994-4003; Chao, et al. Mol Ther. 2000; 2:619-623. AAV-3 has been shown to be superior for the transduction of megakaryocytes. Handa, et al. J Gen Virol. 2000; 81:2077-2084. AAV5 and AAV6 infect apical airway cells efficiently. Zabner, et al. J Virol. 2000; 74:3852-3858; Halbert, et al. J Virol. 2001; 75:6615-6624. AAV2, AAV4, and AAV5 transduce different types of cells in the central nervous system. Davidson, et al. Proc Nat Acad Sci USA. 2000; 97:3428-3432. AAV8 and AAV5 can transduce liver cells better than AAV-2. AAV-5 based vectors transduced certain cell types (cultured airway epithelial cells, cultured striated muscle cells and cultured human umbilical vein endothelial cells) at a higher efficiency than AAV2, while both AAV2 and AAV5 showed poor transduction efficiencies for NIH 3T3, skbr3 and t-47D cell lines. Gao, et al. Proc Natl Acad Sci USA. 2002; 99:11854-11859; Mingozzi, et al. J Virol. 2002; 76:10497-10502. WO 99/61601. AAV4 was found to transduce rat retina most efficiently, followed by AAV5 and AAV1. Rabinowitz, et al. J Virol. 2002; 76:791-801; Weber, et al. Mol Ther. 2003; 7:774-781. In summary, AAV1, AAV2, AAV4, AAV5, AAV8, and AAV9 show tropism for CNS tissues. AAV1, AAV8, and AAV9 show tropism for heart tissues. AAV2 exhibits tropism for kidney tissue. AAV7, AAV8, and AAV9 exhibit tropism for liver tissue. AAV4, AAV5, AAV6, and AAV9 exhibits tropism for lung tissue. AAV8 exhibits tropism for pancreas cells. AAV3, AAV5, and AAV8 show tropism for photoreceptor cells. AAV1, AAV2, AAV4, AAV5, and AAV8 exhibit tropism for retinal pigment epithelium (RPE) cells. AAV1, AAV6, AAV7, AAV8, and AAV9 show tropism for skeletal muscle.

Further modification to the virus can be performed to enhance the efficiency of gene transfer, for example, by improving the tropism of each serotype. One approach is to swap domains from one serotype capsid to another, and thus create hybrid vectors with desirable qualities from each parent. As the viral capsid is responsible for cellular receptor binding, the understanding of viral capsid domain(s) critical for binding is important. Mutation studies on the viral capsid (mainly on AAV2) performed before the availability of the crystal structure were mostly based on capsid surface functionalization by adsorption of exogenous moieties, insertion of peptide at a random position, or comprehensive mutagenesis at the amino acid level. Choi, et al. Curr Gene Ther. 2005 June; 5(3): 299-310, describe different approaches and considerations for hybrid serotypes.

Capsids from other AAV serotypes offer advantages in certain in vivo applications over rAAV vectors based on the AAV2 capsid. First, the appropriate use of rAAV vectors with particular serotypes may increase the efficiency of gene delivery in vivo to certain target cells that are poorly infected, or not infected at all, by AAV2 based vectors. Secondly, it may be advantageous to use rAAV vectors based on other AAV serotypes if re-administration of rAAV vector becomes clinically necessary. It has been demonstrated that re-administration of the same rAAV vector with the same capsid can be ineffective, possibly due to the generation of neutralizing antibodies generated to the vector. Xiao, et al. 1999; Halbert, et al. 1997. This problem may be avoided by administration of a rAAV particle whose capsid is composed of proteins from a different AAV serotype, not affected by the presence of a neutralizing antibody to the first rAAV vector. Xiao, et al. 1999. For the above reasons, recombinant AAV vectors constructed using cap genes from serotypes including and in addition to AAV2 are desirable. It will be recognized that the construction of recombinant HSV vectors similar to rHSV but encoding the cap genes from other AAV serotypes, e.g., AAV1, AAV2, AAV3, AAV5 to AAV9, is achievable using the methods described herein to produce rHSV. In certain preferred embodiments of the present disclosure as described herein, recombinant AAV vectors constructed using cap genes from different AAV are preferred. The significant advantages of construction of these additional rHSV vectors are ease and savings of time, compared with alternative methods used for the large-scale production of rAAV. In particular, the difficult process of constructing new rep and cap inducible cell lines for each different capsid serotypes is avoided.

In certain preferred embodiments of the present disclosure as described herein, recombinant AAV vectors constructed using cap genes encoding variant AAV capsid polypeptides which exhibit increased tropism and/or transduction in inner ear tissues or cell, e.g., as compared to non-variant AAV capsids, are preferred. Such variant variant AAV capsid polypeptides are described herein. Exemplary variant AAV capsid polypeptides are provided in Table 1.

### Variant AAV Capsid Polypeptides

The disclosure generally provides variant adeno-associated virus (AAV) capsid polypeptides which exhibit increased tropism and/or transduction in inner ear tissues or cell, e.g., as compared to non-variant AAV capsid polypeptides, and methods for use in the treatment or prevention of hearing, e.g., hearing loss associated with deficiency of a gene.

According to some embodiments, the variant AAV capsid polypeptide is selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; and a variant AAV12 capsid polypeptide. According to some embodiments, the variant AAV capsid polypeptide is a variant AAV2 capsid polypeptide.

According to some embodiments, the variant AAV capsid polypeptide comprises an amino acid sequence listed in Table 1, or an amino acid sequence having at least about 85%, 90%, 95%, or 99% sequence identity thereto, optionally, wherein the AAV capsid is selected from the group consisting of a VP1, VP2, or VP3 capsid polypeptide.

According to some embodiments, the variant AAV capsid polypeptide comprises an amino acid sequence having one or more amino acid substitutions, insertions, and/or deletions relative to a wildtype AAV2 capsid polypeptide (SEQ ID NO: 18), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730.

According to some embodiments, the variant AAV capsid polypeptide comprises an amino acid sequence having one or more amino acid substitutions relative to a wildtype AAV2 capsid polypeptide (SEQ ID NO: 18) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.

According to some embodiments, the variant AAV capsid polypeptide comprises: (i) an amino acid sequence of any one of SEQ ID NOs: 27, 29, 31, 33, or 35; (ii) an amino acid sequence having at least about 85%, 90%, 95%, or 99% sequence identity to any one of SEQ ID NOs: 27, 29, 31, 33, or 35; or (iii) an amino acid sequence encoded by the nucleic acid sequence of any one of SEQ ID NOs: 26, 28, 30, 32, or 34. According to some embodiments, the variant AAV capsid polypeptide comprises the amino acid sequence of SEQ ID NO: 27. According to some embodiments, the variant AAV capsid polypeptide comprises the amino acid sequence of SEQ ID NO: 29. According to some embodiments, the variant AAV capsid polypeptide comprises the amino acid sequence of SEQ ID NO: 31. According to some embodiments, the variant AAV capsid polypeptide comprises the amino acid sequence of SEQ ID NO: 33. According to some embodiments, the variant AAV capsid polypeptide comprises the amino acid sequence of SEQ ID NO: 35.

According to some embodiments, the variant AAV capsid polypeptide results in an increased level of rAAV tropism in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold as compared to a non-variant AAV capsid polypeptide. In certain embodiments, the variant AAV capsid polypeptide results in an increased level of rAAV tropism in an inner ear tissue or cell selected from the group consisting of a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, and/or a border cell.

According to some embodiments, the variant AAV capsid polypeptide results in an increased level of rAAV transduction efficiency in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to a non-variant AAV capsid polypeptide. In certain embodiments, the variant AAV capsid polypeptide results in an increased level of rAAV transduction efficiency in an inner ear tissue or cell selected from the group consisting of a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, and/or a border cell, an inner cochlear hair cell, an outer cochlear hair cell, a spiral ganglion neuron, a vestibular hair cell, a vestibular support cell, and/or a vestibular ganglion neuron.

Making Recombinant AAV (rAAV) Vectors

The production, purification, and characterization of the rAAV vectors of the present disclosure may be carried out using any of the many methods known in the art. According to some embodiments, the rAAV vetors encode an AAV variant capsid polypeptide as described herein (e.g., Table 1) which exhibits increased tropism and/or transduction in inner ear tissues or cell, e.g., as compared to non-variant AAV capsid polypeptides. For reviews of laboratory-scale production methods, see, e.g., Clark R K, Recent advances in recombinant adeno-associated virus vector production. Kidney Int. 61s:9-15 (2002); Choi V W et al., Production of recombinant adeno-associated viral vectors for in vitro and in vivo use. Current Protocols in Molecular Biology 16.25.1-16.25.24 (2007) (hereinafter Choi et al.); Grieger J C & Samulski R J, Adeno-associated virus as a gene therapy vector: Vector development, production, and clinical applications. Adv Biochem Engin/Biotechnol 99:119-145 (2005) (hereinafter Grieger & Samulski); Heilbronn R & Weger S, Viral Vectors for Gene Transfer: Current Status of Gene Therapeutics, in M. Schäfer-Korting (ed.), Drug Delivery, Handbook of Experimental Pharmacology, 197: 143-170 (2010) (hereinafter Heilbronn); Howarth J L et al., Using viral vectors as gene transfer tools. Cell Biol Toxicol 26:1-10 (2010) (hereinafter Howarth). The production methods described below are intended as non-limiting examples.

AAV vector production may be accomplished by cotransfection of packaging plasmids. Heilbronn. The cell line supplies the deleted AAV genes rep and cap and the required helpervirus functions. The adenovirus helper genes, VA-RNA, E2A and E4 are transfected together with the AAV rep and cap genes, either on two separate plasmids or on a single helper construct. A recombinant AAV vector plasmid wherein the AAV capsid genes are replaced with a transgene expression cassette (comprising the gene of interest, e.g., a GJB2 nucleic acid; a promoter; and minimal regulatory elements) bracketed by ITRs, is also transfected. These packaging plasmids are typically transfected into 293 cells, a human cell line that constitutively expresses the remaining required Ad helper genes, E1A and E1B. This leads to amplification and packaging of the AAV vector carrying the gene of interest.

Multiple serotypes of AAV, including 12 human serotypes and more than 100 serotypes from nonhuman primates have now been identified. Howarth et al. Cell Biol Toxicol 26:1-10 (2010). The AAV vectors of the present disclosure may comprise capsid sequences derived from AAV5 of any known serotype. As used herein, a “known serotype” encompasses capsid mutants that can be produced using methods known in the art. Such methods, include, for example, genetic manipulation of the viral capsid sequence, domain swapping of exposed surfaces of the capsid regions of different serotypes, and generation of AAV chimeras using techniques such as marker rescue. See Bowles et al. Marker rescue of adeno-associated virus (AAV) capsid mutants: A novel approach for chimeric AAV production. Journal of Virology, 77(1): 423-432 (2003), as well as references cited therein. Moreover, the AAV vectors of the present disclosure may comprise ITRs derived from AAVs of any known serotype. Preferentially, the ITRs are derived from one of the human serotypes AAV1-AAV12. In some embodiments of the present disclosure, a pseudotyping approach is employed, wherein the genome of one ITR serotype is packaged into a different serotype capsid.

According to some embodiments, the capsid sequences are derived from one of the human serotypes AAV1-AAV12. According to some embodiments, the capsid sequences are derived from serotype AAV2. According to some embodiments, the capsid sequences are derived from an AAV2 variant with high tropism for targeting inner ear tissues or cells, e.g., support cells (e.g., outer hair cells, inner hair cells, hensen's cells, deiters' cells, pillar cells, inner phalangeal cells, outer phalangeal cells/border cells, inner and outer cochlear hair cells, spiral ganglion neurons, vestibular hair cells, vestibular support cells and vestibular ganglion neurons). In certain embodiments, the variant AAV capsid polypeptide results in an increased level of rAAV tropism and/or transduction efficiency in an inner ear tissue or cell selected from the group consisting of a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, a border cell, an inner cochlear hair cell, an outer cochlear hair cell, a spiral ganglion neuron, a vestibular hair cell, a vestibular support cell and/or a vestibular ganglion neuron. Capsids suitable for this purpose comprise AAV2 and AAV2 variants including AAV2-tYF, AAV2-MeB, AAV2-P2V2, AAV2-MeBtYFTV, AAV2-P2V6; as well as AAV5, AAV8, and Anc80L65. According to some embodiments, the variant AAV capsid polypeptide comprises an amino acid sequence listed in Table 1, or an amino acid sequence having at least about 85%, 90%, 95%, or 99% sequence identity thereto.

According to some embodiments, recombinant AAV vectors can be directly targeted by genetic manipulation of the viral capsid sequence, particularly in the looped out region of the AAV three-dimensional structure, or by domain swapping of exposed surfaces of the capsid regions of different serotypes, or by generation of AAV chimeras using techniques such as marker rescue. See Bowles et al. Marker rescue of adeno-associated virus (AAV) capsid mutants: A novel approach for chimeric AAV production. Journal of Virology, 77(1): 423-432 (2003), as well as references cited therein.

One possible protocol for the production, purification, and characterization of recombinant AAV (rAAV) vectors is provided in Choi et al. Generally, the following steps are involved: design a transgene expression cassette, design a capsid sequence for targeting a specific receptor, generate adenovirus-free rAAV vectors, purify and titer. These steps are summarized below and described in detail in Choi et al.

The transgene expression cassette may be a single-stranded AAV (ssAAV) vector or a “dimeric” or self-complementary AAV (scAAV) vector that is packaged as a pseudo-double-stranded transgene. Choi et al.; Howarth et al. Using a traditional ssAAV vector generally results in a slow onset of gene expression (from days to weeks until a plateau of transgene expression is reached) due to the required conversion of single-stranded AAV DNA into double-stranded DNA. In contrast, scAAV vectors show an onset of gene expression within hours that plateaus within days after transduction of quiescent cells. Heilbronn. According to some embodiments, a scAAV is used, where the scAAV has rapid transduction onset and increased stability compared to single stranded AAV. Alternatively, the transgene expression cassette may be split between two AAV vectors, which allows delivery of a longer construct. See e.g., Daya S. and Berns, K. I., Gene therapy using adeno-associated virus vectors. Clinical Microbiology Reviews, 21(4): 583-593 (2008) (hereinafter Daya et al.). A ssAAV vector can be constructed by digesting an appropriate plasmid (such as, for example, a plasmid containing the GJB2 gene) with restriction endonucleases to remove the rep and cap fragments, and gel purifying the plasmid backbone containing the AAVwt-ITRs. Choi et al. Subsequently, the desired transgene expression cassette can be inserted between the appropriate restriction sites to construct the single-stranded rAAV vector plasmid. A scAAV vector can be constructed as described in Choi et al.

Then, a large-scale plasmid preparation (at least 1 mg) of the rAAV vector and the suitable AAV helper plasmid and pXX6 Ad helper plasmid can be purified by double CsCl gradient fractionation. Choi et al. A suitable AAV helper plasmid may be selected from the pXR series, pXR1-pXR5, which respectively permit cross-packaging of AAV2 ITR genomes into capsids of AAV serotypes 1 to 5. The appropriate capsid may be chosen based on the efficiency of the capsid's targeting of the cells of interest, e.g., inner ear tissues and cells. Known methods of varying genome (i.e., transgene expression cassette) length and AAV capsids may be employed to improve expression and/or gene transfer to specific cell types (e.g., retinal cone cells). See, e.g., Yang G S, Virus-mediated transduction of murine retina with adeno-associated virus: Effects of viral capsid and genome size. Journal of Virology, 76(15): 7651-7660. According to some embodiments, the variant AAV capsid polypeptide comprises an amino acid sequence listed in Table 1, or an amino acid sequence having at least about 85%, 90%, 95%, or 99% sequence identity thereto.

Next, 293 cells are transfected with pXX6 helper plasmid, rAAV vector plasmid, and AAV helper plasmid. Choi et al. Subsequently the fractionated cell lysates are subjected to a multistep process of rAAV purification, followed by either CsCl gradient purification or heparin sepharose column purification. The production and quantitation of rAAV virions may be determined using a dot-blot assay. In vitro transduction of rAAV in cell culture can be used to verify the infectivity of the virus and functionality of the expression cassette.

In addition to the methods described in Choi et al., various other transfection methods for production of AAV may be used in the context of the present disclosure. For example, transient transfection methods are available, including methods that rely on a calcium phosphate precipitation protocol.

In addition to the laboratory-scale methods for producing rAAV vectors, the present disclosure may utilize techniques known in the art for bioreactor-scale manufacturing of AAV vectors, including, for example, Heilbronn; Clement, N. et al. Large-scale adeno-associated viral vector production using a herpesvirus-based system enables manufacturing for clinical studies. Human Gene Therapy, 20: 796-606.

Advances toward achieving the desired goal of scalable production systems that can yield large quantities of clinical grade rAAV vectors have largely been made in production systems that utilize transfection as a means of delivering the genetic elements needed for rAAV production in a cell. For example, removal of contaminating adenovirus helper has been circumvented by replacing adenovirus infection with plasmid transfection in a three-plasmid transfection system in which a third plasmid comprises nucleic acid sequences encoding adenovirus helper proteins (Xiao, et al. 1998), Improvements in two-plasmid transfection systems have also simplified the production process and increased rAAV vector production efficiency (Grimm, et al. 1998).

Several strategies for improving yields of rAAV from cultured mammalian cells are based on the development of specialized producer cells created by genetic engineering. In one approach, production of rAAV on a large scale has been accomplished by using genetically engineered “proviral” cell lines in which an inserted AAV genome can be “rescued” by infecting the cell with helper adenovirus or HSV. Proviral cell lines can be rescued by simple adenovirus infection, offering increased efficiency relative to transfection protocols.

A second cell-based approach to improving yields of rAAV from cells involves the use of genetically engineered “packaging” cell lines that harbor in their genomes either the AAV rep and cap genes, or both the rep-cap and the ITR-gene of interest (Qiao, et al. 2002). In the former approach, in order to produce rAAV, a packaging cell line is either infected or transfected with helper functions, and with the AAV ITR-GOI elements. The latter approach entails infection or transfection of the cells with only the helper functions. Typically, rAAV production using a packaging cell line is initiated by infecting the cells with wild-type adenovirus, or recombinant adenovirus. Because the packaging cells comprise the rep and cap genes, it is not necessary to supply these elements exogenously.

rAAV yields from packaging cell lines have been shown to be higher than those obtained by proviral cell line rescue or transfection protocols.

Improved yields of rAAV have been made using approaches based on delivery of helper functions from herpes simplex virus (HSV) using recombinant HSV amplicon systems. Although modest levels of rAAV vector yield, of the order of 150-500 viral genomes (vg) per cell, were initially repotted (Conway, et al. 1997), more recent improvements in rHSV amplicon-based systems have provided substantially higher yields of rAAV v.g. and infectious particles (ip) per cell (Feudner, et al. 2002). Amplicon systems are inherently replication-deficient; however the use of a “gutted” vector, replication-competent (rcHSV), or replication-deficient rHSV still introduces immunogenic HSV components into rAAV production systems. Therefore, appropriate assays for these components and corresponding purification protocols for their removal are implemented.

In addition to these methods, methods for producing recombinant AAV viral particles in a mammalian cell are described herein comprising co-infecting a mammalian cell capable of growing in suspension with a first recombinant herpesvirus comprising a nucleic acid sequence encoding an AAV rep and an AAV cap gene each operably linked to a promoter, and a second recombinant herpesvirus comprising a gene, e.g., a GJB2 gene, and a promoter operably linked to said gene, e.g., GJB2 gene, flanked by AAV inverted terminal repeats to facilitate packaging of the gene of interest, and allowing the virus to infect the mammalian cell, thereby producing recombinant AAV viral particles in a mammalian cell. In some embodiments, the AAV cap gene encodes an AAV variant capsid polypeptide as described herein (e.g., Table 1) which exhibits increased tropism in inner ear tissues or cell, e.g., as compared to non-variant AAV capsid polypeptides.

Any type of mammalian cell that is capable of supporting replication of herpesvirus is suitable for use according to the methods of the present disclosure as described herein. Accordingly, the mammalian cell can be considered a host cell for the replication of herpesvirus as described in the methods herein. Any cell type for use as a host cell is contemplated by the present disclosure, as long as the cell is capable of supporting replication of herpesvirus. Examples of suitable genetically unmodified mammalian cells include but are not limited to cell lines such as HEK-293 (293), Vero, RD, BHK-21, HT-1080, A549, Cos-7, ARPE-19, and MRC-5.

The host cells used in the various embodiments of the present disclosure may be derived, for example, from mammalian cells such as human embryonic kidney cells or primate cells. Other cell types might include, but are not limited to BHK cells, Vero cells, CHO cells or any eukaryotic cells for which tissue culture techniques are established as long as the cells are herpesvirus permissive. The term “herpesvirus permissive” means that the herpesvirus or herpesvirus vector is able to complete the entire intracellular virus life cycle within the cellular environment. In certain embodiments, methods as described occur in the mammalian cell line BHK, growing in suspension. The host cell may be derived from an existing cell line, e.g., from a BHK cell line, or developed de novo.

The methods for producing a rAAV gene construct described herein include also a recombinant AAV viral particle produced in a mammalian cell by the method comprising co-infecting a mammalian cell capable of growing in suspension with a first recombinant herpesvirus comprising a nucleic acid encoding an AAV rep and an AAV cap gene each operably linked to a promoter; and (ii) a second recombinant herpesvirus comprising a gene, e.g., a GJB2, and a promoter operably linked to said gene, e.g., GJB2 gene; and allowing the virus to infect the mammalian cell, and thereby producing recombinant AAV viral particles in a mammalian cell. As described herein, the herpesvirus is a virus selected from the group consisting of: cytomegalovirus (CMV), herpes simplex (HSV) and varicella zoster (VZV) and epstein barr virus (EBV). The recombinant herpesvirus is replication defective. According to some embodiments, the AAV cap gene has a serotype selected from the group consisting of AAV1, AAV2, AAV3, AAV4, AAV5, AAV6, AAV7, AAV8, AAV9, AAV10, AAV11, AAV12, AAVrh8, AAVrh10, Anc80L65, including variants or hybrids (e.g., capsid hybrids of two or more serotypes). According to some embodiments, the AAV cap gene encodes an AAV variant capsid polypeptide as described herein (e.g., Table 1) which exhibits increased tropism in inner ear tissues or cell, e.g., as compared to non-variant AAV capsid polypeptides.

U.S. Patent Application Publication No. 2007/0202587, incorporated by reference in its entirety herein, describes required elements of rAAV Production Systems. Recombinant AAV is produced in vitro by introduction of gene constructs into cells known as producer cells. Known systems for production of rAAV employ three fundamental elements: (1) a gene cassette containing the gene of interest, (2) a gene cassette containing AAV rep and cap genes and (3) a source of “helper” virus proteins.

The first gene cassette is constructed with the gene of interest flanked by inverted terminal repeats (ITRs) from AAV. ITRs function to direct integration of the gene of interest into the host cell genome and play a significant role in encapsidation of the recombinant genome. Hermonat and Muzyczka, 1984; Samulski et al. 1983. The second gene cassette contains rep and cap, AAV genes encoding proteins needed for replication and packaging of rAAV. The rep gene encodes four proteins (Rep 78, 68, 52 and 40) required for DNA replication. The cap genes encode three structural proteins (VP1, VP2, and VP3) that make up the virus capsid. Muzyczka and Berns, 2001.

The third element is required because AAV does not replicate on its own. Helper functions are protein products from helper DNA viruses that create a cellular environment conducive to efficient replication and packaging of rAAV. Traditionally, adenovirus (Ad) has been used to provide helper functions for rAAV, but herpesviruses can also provide these functions as discussed herein.

Production of rAAV vectors for gene therapy is carried out in vitro, using suitable producer cell lines such as BHK cells grown in suspension. Other cell lines suitable for use in the present disclosure include HEK-293 (293), Vero, RD, BHK-21, HT-1080, A549, Cos-7, ARPE-19, and MRC-5.

Any cell type can be used as a host cell, as long as the cell is capable of supporting replication of a herpesvirus. One of skill in the art would be familiar with the wide range of host cells that can be used in the production of herpesvirus from host cells. Examples of suitable genetically unmodified mammalian host cells, for example, may include but are not limited to cell lines such as HEK-293 (293), Vero, RD, BHK-21, HT-1080, A549, Cos-7, ARPE-19, and MRC-5.

A host cell may be adapted for growth in suspension culture. The host cells may be Baby Hamster Kidney (BHK) cells. BHK cell line grown in suspension is derived from an adaptation of the adherent BHK cell line. Both cell lines are available commercially.

One strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes. To introduce these various elements into the cells, the cells are infected with Ad as well as transfected with the two plasmids. The gene products provided by Ad are encoded by the genes Ela, E1b, E2a, E4orf6, and Va. Samulski et al. 1998: Hauswirth et al. 2000; Muzyczka and Burns, 2001. Alternatively, in more recent protocols, the Ad infection step can be replaced by transfection with an adenovirus “helper plasmid” containing the VA, E2A and E4 genes. Xiao et al. 1998; Matsushita, et al. 1998.

While Ad has been used conventionally as the helper virus for rAAV production, other DNA viruses, such as herpes simplex virus type 1 (HSV-1) can be used as well. The minimal set of HSV-1 genes required for AAV2 replication and packaging has been identified, and includes the early genes UL5, UL8, UL52 and UL29. Muzyczka and Burns, 2001. These genes encode components of the HSV-1 core replication machinery, i.e., the helicase, primase, primase accessory proteins, and the single-stranded DNA binding protein. Knipe, 1989; Weller, 1991. This rAAV helper property of HSV-1 has been utilized in the design and construction of a recombinant herpes virus vector capable of providing helper virus gene products needed for rAAV production. Conway et al. 1999.

Production of rAAV vectors for gene therapy is carried out in vitro, using suitable producer cell lines such as BHK cells grown in suspension. Other cell lines suitable for use in the present disclosure include HEK-293 (293), Vero, RD, BHK-21, HT-1080, A549, Cos-7, ARPE-19, and MRC-5.

Any cell type can be used as a host cell, as long as the cell is capable of supporting replication of a herpesvirus. One of skill in the art would be familiar with the wide range of host-cells that can be used in the production of herpesvirus from host cells. Examples of suitable genetically unmodified mammalian host cells, for example, may include but are not limited to cell lines such as HEK-293 (293), Vero, RD, BHK-21, HT-1080, A549, Cos-7, ARPE-19, and MRC-5.

A host cell may be adapted for growth in suspension culture. In certain embodiments of the present disclosure, the host cells are Baby Hamster Kidney (BHK) cells. BHK cell line grown in suspension is derived from an adaptation of the adherent BHK cell line. Both cell lines are available commercially.

rHSV-Based rAAV Manufacturing Process

Methods for the production of recombinant AAV viral particles in cells growing in suspension are described herein. According to some embodiments, the AAV particles comprise an AAV variant capsid polypeptide as described herein (e.g., Table 1) which exhibits increased tropism and/or transduction in inner ear tissues or cell, e.g., as compared to non-variant AAV capsid polypeptides. Suspension or non-anchorage dependent cultures from continuous established cell lines are the most widely used means of large scale production of cells and cell products. Large scale suspension culture based on fermentation technology has clear advantages for the manufacturing of mammalian cell products. Homogeneous conditions can be provided in the bioreactor which allows for precise monitoring and control of temperature, dissolved oxygen, and pH, and ensure that representative samples of the culture can be taken. The rHSV vectors used are readily propagated to high titer on permissive cell lines both in tissue culture flasks and bioreactors, and provided a production protocol amenable to scale-up for virus production levels necessary for clinical and market production.

Cell culture in stirred tank bioreactors provides very high volume-specific culture surface area and has been used for the production of viral vaccines (Griffiths, 1986). Furthermore, stirred tank bioreactors have industrially been proven to be scalable. One example is the multiplate CELL CUBE cell culture system. The ability to produce infectious viral vectors is increasingly important to the pharmaceutical industry, especially in the context of gene therapy.

Growing cells according to methods described herein may be done in a bioreactor that allows for large scale production of fully biologically-active cells capable of being infected by the Herpes vectors of the present disclosure. Bioreactors have been widely used for the production of biological products from both suspension and anchorage dependent animal cell cultures. Most large-scale suspension cultures are operated as batch or fed-batch processes because they are the most straightforward to operate and scale up. However, continuous processes based on chemostat or perfusion principles are available. The bioreactor system may be set up to include a system to allow for media exchange. For example, filters may be incorporated into the bioreactor system to allow for separation of cells from spent media to facilitate media exchange. In some embodiments of the present methods for producing Herpes virus, media exchange and perfusion is conducted beginning on a certain day of cell growth. For example, media exchange and perfusion can begin on day 3 of cell growth. The filter may be external to the bioreactor, or internal to the bioreactor.

A method for producing recombinant AAV viral particles may comprise: co-infecting a suspension cell with a first recombinant herpesvirus comprising a nucleic acid encoding an AAV rep and an AAV cap gene each operably linked to a promoter; and a second recombinant herpesvirus comprising a gene construct, e.g., a GJB2 gene construct, and a promoter operably linked to said gene of interest; and allowing the cell to produce the recombinant AAV viral particles, thereby producing the recombinant AAV viral particles. The cell may be HEK-293 (293), Vero, RD, BHK-21, HT-1080, A549, Cos-7, ARPE-19, and MRC-5. According to some embodiments, the cap gene may be selected from an AAV with a serotype selected from the group consisting of AAV1, AAV2, AAV-, AAV4, AAV5, AAV6, AAV7, AAV8, AAV9, AAV10, AAV11, AAV12, AAVrh8, AAVrh10, Anc80L65, including variants or hybrids thereof (e.g., capsid hybrids of two or more serotypes). According to some embodiments, the AAV cap gene encodes an AAV variant capsid polypeptide as described herein (e.g., Table 1) which exhibits increased tropism and/or transduction in inner ear tissues or cell, e.g., as compared to non-variant AAV capsid polypeptides. The cell may be infected at a combined multiplicity of infection (MOI) of between 3 and 14. The first herpesvirus and the second herpesvirus may be viruses selected from the group consisting of: cytomegalovirus (CMV), herpes simplex (HSV) and varicella zoster (VZV) and epstein barr virus (EBV). The herpesvirus may be replication defective. The co-infection may be simultaneous.

A method for producing recombinant AAV viral particles in a mammalian cell may comprise co-infecting a suspension cell with a first recombinant herpesvirus comprising a nucleic acid encoding an AAV rep and an AAV cap gene each operably linked to a promoter; and a second recombinant herpesvirus comprising a gene construct, e.g., a GJB2 gene construct, and a promoter operably linked to said gene construct, e.g., GJB2 gene construct; and allowing the cell to propagate, thereby producing the recombinant AAV viral particles, whereby the number of viral particles produced is equal to or greater than the number of viral particles grown in an equal number of cells under adherent conditions. The cell may be HEK-293 (293), Vero, RD, BHK-21, HT-1080, A549, Cos-7, ARPE-19, and MRC-5. The cap gene may be selected from an AAV with a serotype selected from the group consisting of AAV1, AAV2, AAV3, AAV4, AAV5, AAV6, AAV7, AAV8, AAV9, AAV10, AAV11, AAV12, AAVrh8, AAVrh10, Anc80L65, including variants or hybrids thereof (e.g., capsid hybrids of two or more serotypes). According to some embodiments, the AAV cap gene encodes an AAV variant capsid polypeptide as described herein (e.g., Table 1) which exhibits increased tropism and/or transduction in inner ear tissues or cell, e.g., as compared to non-variant AAV capsid polypeptides. The cell may be infected at a combined multiplicity of infection (MOI) of between 3 and 14. The first herpesvirus and the second herpesvirus may be viruses selected from the group consisting of: cytomegalovirus (CMV), herpes simplex (HSV) and varicella zoster (VZV) and epstein barr virus (EBV). The herpesvirus may be replication defective. The co-infection may be simultaneous.

A method for delivering a nucleic acid sequence encoding a therapeutic protein to a suspension cell, the method comprising: co-infecting the BHK cell with a first recombinant herpesvirus comprising a nucleic acid encoding an AAV rep and an AAV cap gene each operably linked to a promoter; and a second herpesvirus comprising a gene construct, e.g., a GJB2 gene construct, wherein the gene of interest comprises a therapeutic protein coding sequence, and a promoter operably linked to said gene, e.g., GJB2 gene; and wherein said cell is infected at a combined multiplicity of infection (MOI) of between 3 and 14; and allowing the virus to infect the cell and express the therapeutic protein, thereby delivering the nucleic acid sequence encoding the therapeutic protein to the cell. The cell may be HEK-293 (293), Vero, RD, BHK-21, HT-1080, A549, Cos-7, ARPE-19, and MRC-5. See, e.g., U.S. Pat. No. 9,783,826. According to some embodiments, the AAV cap gene encodes an AAV variant capsid polypeptide as described herein (e.g., Table 1) which exhibits increased tropism and/or transduction in inner ear tissues or cell, e.g., as compared to non-variant AAV capsid polypeptides.

### Methods of Treatment

AAV and Gene Therapy

Gene therapy refers to treatment of inherited or acquired diseases by replacing, altering, or supplementing a gene responsible for the disease. It is achieved by introduction of a corrective gene or genes into a host cell, generally by means of a vehicle or vector. According to some embodiments, the rAAV described herein comprise AAV variant capsid polypeptide (e.g., Table 1) which exhibits increased tropism and/or transduction in inner ear tissues or cell, e.g., as compared to non-variant AAV capsid polypeptides.

According to some embodiments, the GJB2 AAV construct provides a gene therapy vehicle for the treatment of DFNB1 deafness phenotype. The GJB2 AAV gene therapy construct and methods of use described herein provides a therapy for DFNB1 deafness, a long-felt unmet need as there are no gene therapy-based treatments available for patients.

Methods of Treating Hearing Loss

Methods are provided herein that can be used to treat a hearing disorder or to prevent hearing loss (or further hearing loss) in a subject. Delivery of one or more of the nucleic acids described herein to cells within the inner ear, e.g., in the cochlea (or cells of the cochlea or cochlear cells) can be used to treat hearing disorders, which are typically defined by partial hearing loss or complete deafness.

According to some embodiments, methods are provided herein that employ GJB2 AAV-based gene therapy for treating non-syndromic hearing loss and deafness characterized by congenital progressive and non-progressive mild-to-profound sensorineural hearing impairment. The GJB2 AAV gene therapy construct and methods of use described herein provide an example of a long term (e.g., lifelong) therapy for correcting congenital deafness by gene supplementation. Importantly, the GJB2 AAV gene therapy construct and methods of use described herein would preserve natural hearing, while cochlear implants do not.

The methods described herein allow for the production of recombinant AAV viral particles in a mammalian cell comprises co-infecting a mammalian cell capable of growing in suspension with a first recombinant herpesvirus and a second recombinant herpesvirus comprising a GJB2 gene construct that has therapeutic value in the treatment of genetic deafness.

GJB2 codes for the major gap junction protein Connexin 26 (Cx26), which, in association with other gap junction proteins, provides an extensive network allowing for intercellular coupling among non-sensory cells in the cochlea. Furthermore, GJB2/Cx26 can play a significant role in the formation of a gap junction network required for normal hearing by maintaining potassium gradient homeostasis in the Organ of Corti. Individuals with autosomal recessive mutations in GJB2 manifest the DFNB1 deafness phenotype, and this accounts for nearly half of all cases of genetic hearing loss, with a prevalence of about 2-3 in every 1000 births. These are manifested as homozygous or compound heterozygous mutations (del Castillo & del Castillo, Front Mol Neurosci. 2017; 10: 428). In addition, there are heterozygous carriers that are at risk for accelerated age-related hearing loss (del Castillo & del Castillo, Front Mol Neurosci. 2017; 10: 428).

In one aspect, the present disclosure relates to a novel rAAV-based gene therapy for treating or preventing genetic hearing loss due to GJB2 mutation, accounting for approximately 45% of all cases of congenital deafness. In addition, the disclosure relates to the treatment or prevention of hearing loss that is associated with heterozygous mutations. The rAAV constructs detailed in this disclosure will correspond to pre-lingual or post-lingual therapies for the prevention or treatment of both autosomal recessive GJB2 mutants (DFNB1) and autosomal dominant GJB2 mutants (DFNA3A), and administered by whatever method is necessary for intracochlear delivery. The gene constructs described herein may be used in methods and/or compositions to treat and/or prevent DFNB1 deafness.

According to some embodiments, the GJB2 AAV gene therapy is administered to a subject that has already developed significant hearing loss. According to some embodiments, the GJB2 AAV gene therapy is administered before the subject has developed hearing loss. According to some embodiments, the subject is diagnosed with DFNB1 by molecular genetic testing to identify deafness-causing mutations in GJB2. According to some embodiments, the subject has a family member with nonsyndromic hearing loss and deafness. According to some embodiments, the subject is a child. According to some embodiments, the subject is an infant.

The rAAV constructs described herein transduce inner ear cells and tissues, e.g., cochlear cells, with greater efficiency than do conventional AAV vectors. According to some embodiments, the compositions and methods described herein enable the highly efficient delivery of nucleic acids to inner ear cells, e.g., cochlear cells. According to some embodiments, the compositions and methods described herein enable the delivery to, and expression of, a transgene in at least 30% (e.g., at least 30, 35, 40, 45, 50, 55, 60, 65, 70, 75, 80, 85, 90, 91, 92, 93, 94, 95, 96, 97, 98, or 99%) of inner ear cells, e.g., cochlear cells. According to some embodiments, the compositions and methods described herein enable the delivery to, and expression of, a transgene in at least 50% (e.g., at least 50, 55, 60, 65, 70, 75, 80, 85, 90, 91, 92, 93, 94, 95, 96, 97, 98, or 99%) of inner ear cells, e.g., cochlear cells. According to some embodiments, the compositions and methods described herein enable the delivery to, and expression of, a transgene in at least 70% (e.g., at least 70, 75, 80, 85, 90, 91, 92, 93, 94, 95, 96, 97, 98, or 99%) of inner ear cells, e.g., cochlear cells. According to some embodiments, the compositions and methods described herein enable the delivery to, and expression of, a transgene in at least 90% (e.g., at least 90, 91, 92, 93, 94, 95, 96, 97, 98, or 99%) of inner ear cells, e.g., cochlear cells.

The rAAV constructs described herein transduce auditory hair cells, e.g., inner hair cells and/or outer hair cells, with greater efficiency than do conventional AAV vectors. According to some embodiments, the compositions and methods described herein enable the highly efficient delivery of nucleic acids to auditory hair cells, e.g., inner hair cells and/or outer hair cells. According to some embodiments, the compositions and methods described herein enable the delivery to, and expression of, a transgene in at least 30% (e.g., at least 30, 35, 40, 45, 50, 55, 60, 65, 70, 75, 80, 85, 90, 91, 92, 93, 94, 95, 96, 97, 98, or 99%) of inner hair cells or delivery to, and expression in, at least 30% (e.g., at least 30, 35, 40, 45, 50, 55, 60, 65, 70, 75, 80, 85, 90, 91, 92, 93, 94, 95, 96, 97, 98, or 99) of outer hair cells. According to some embodiments, the compositions and methods described herein enable the delivery to, and expression of, a transgene in at least 50% (e.g., at least 50, 55, 60, 65, 70, 75, 80, 85, 90, 91, 92, 93, 94, 95, 96, 97, 98, or 99%) of inner hair cells or delivery to, and expression in, at least 50% (e.g., at least 50, 55, 60, 65, 70, 75, 80, 85, 90, 91, 92, 93, 94, 95, 96, 97, 98, or 99) of outer hair cells. According to some embodiments, the compositions and methods described herein enable the delivery to, and expression of, a transgene in at least 70% (e.g., at least 70, 75, 80, 85, 90, 91, 92, 93, 94, 95, 96, 97, 98, or 99%) of inner hair cells or delivery to, and expression in, at least 70% (e.g., at least 70, 75, 80, 85, 90, 91, 92, 93, 94, 95, 96, 97, 98, or 99) of outer hair cells. According to some embodiments, the compositions and methods described herein enable the delivery to, and expression of, a transgene in at least 90% (e.g., at least 90, 91, 92, 93, 94, 95, 96, 97, 98, or 99%) of inner hair cells or delivery to, and expression in, at least 90% (e.g., at least 90, 91, 92, 93, 94, 95, 96, 97, 98, or 99) of outer hair cells.

The rAAV constructs described herein transduce inner ear supporting cells with greater efficiency than do conventional AAV vectors. According to some embodiments, the compositions and methods described herein enable the highly efficient delivery of nucleic acids to inner ear supporting cells. According to some embodiments, the compositions and methods described herein enable the delivery to, and expression of, a transgene in at least 30% (e.g., at least 30, 35, 40, 45, 50, 55, 60, 65, 70, 75, 80, 85, 90, 91, 92, 93, 94, 95, 96, 97, 98, or 99%) of inner ear supporting cells. According to some embodiments, the compositions and methods described herein enable the delivery to, and expression of, a transgene in at least 50% (e.g., at least 50, 55, 60, 65, 70, 75, 80, 85, 90, 91, 92, 93, 94, 95, 96, 97, 98, or 99%) of inner ear supporting cells. According to some embodiments, the compositions and methods described herein enable the delivery to, and expression of, a transgene in at least 70% (e.g., at least 70, 75, 80, 85, 90, 91, 92, 93, 94, 95, 96, 97, 98, or 99%) of inner ear supporting cells. According to some embodiments, the compositions and methods described herein enable the delivery to, and expression of, a transgene in at least 90% (e.g., at least 90, 91, 92, 93, 94, 95, 96, 97, 98, or 99%) of inner ear supporting cells.

According to some embodiments, the nucleic acid sequences described herein are directly introduced into a cell, where the nucleic acid sequences are expressed to produce the encoded product, prior to administration in vivo of the resulting recombinant cell. This can be accomplished by any of numerous methods known in the art, e.g., by such methods as electroporation, lipofection, calcium phosphate mediated transfection.

Accordingly, methods are provided herein for treating or preventing hearing loss associated with deficiency of a gene, such as GJB2. In some embodiments, the method comprises administering to a subject in need thereof an effective amount of a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding the gene.

Additionally, methods are provided herein for delivering a nucleic acid sequence encoding a gene, such as GJB2, associated with hearing loss to an inner ear tissue or cell. In some embodiments, the method comprises administering to a subject in need thereof an effective amount of a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding the gene.

The methods provided herein can result in increased expression of the gene in the inner ear tissues or cells. According to some embodiments, the methods described herein increase the expression of the gene, e.g., GJB2, at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to normal expression of the gene. In some embodiments, the methods can result in the overexpression of the gene, e.g., GJB2, in the inner ear tissues or cells.

The methods provided herein can result in decreased level of rAAV neutralizing antibody (NAb) titers. According to some embodiments, the methods described herein decrease the level of rAAV Nab titers by at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a control level.

The methods provided herein can result in a decreased level of inner ear inflammation and/or toxicity. According to some embodiments, the methods described herein decrease the level of inner ear inflammation and/or toxicity by at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of inner ear inflammation or toxicity prior to administration. In some embodiments, the methods provided herein can result in a delay in progression of inner ear inflammation or toxicity, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to progression of inner ear inflammation or toxicity prior to administration. In certain embodiments, the level of inner ear inflammation and/or toxicity is a level of inner ear inflammation and/or toxicity associated with administration of a AAV virion comprising a non-variant AAV capsid. In certain embodiments, the level of inner ear inflammation and/or toxicity is a level of inner ear inflammation and/or toxicity associated with an underlying disease and/or disorder characterized by hearing loss in a subject.

The methods provided herein can result in a decreased level of hair cell loss, degeneration, and/or death. According to some embodiments, the methods described herein decrease the level of hair cell loss, degeneration, and/or death by at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of hair cell loss, degeneration, and/or death prior to administration.

The methods provided herein can result in a decreased level of spiral ganglion neuron loss, degeneration, and/or death. According to some embodiments, the methods described herein decrease the level of spiral ganglion neuron loss, degeneration, and/or death by at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of spiral ganglion neuron loss, degeneration, and/or death prior to administration.

The methods provided herein can result in various improvements in hearing. Improvements in hearing can be evaluated in numerous ways known in the art. For example, physiologic tests may be used to objectively determine the functional status of the auditory system and can be performed at any age. Exemplary physiologic tests include the following: auditory brain stem response testing (ABR, also known as BAER, BSER), auditory steady-state response testing (ASSR), evoked otoacoustic emissions (EOAEs), and immittance testing (tympanometry, acoustic reflex thresholds, acoustic reflex decay).

Auditory brain stem response testing (ABR, also known as BAER, BSER) uses a stimulus (clicks or pure tones) to evoke electrophysiologic responses, which originate in the eighth cranial nerve and auditory brain stem and are recorded with surface electrodes.

Auditory steady-state response testing (ASSR) is like ABR in that both are auditory evoked potentials and they are measured in similar ways. ASSR uses an objective, statistics-based mathematical detection algorithm to detect and define hearing thresholds. ASSR can be obtained using broadband or frequency-specific stimuli and can offer hearing threshold differentiation in the severe-to-profound range. It is frequently used to give frequency-specific information that ABR does not give. Test frequencies of 500, 1000, 2000, and 4000 Hz are commonly used. In some embodiments, the methods provided herein result in an improved ASSR response.

Evoked otoacoustic emissions (EOAEs) are sounds originating within the cochlea that are measured in the external auditory canal using a probe with a microphone and transducer. EOAEs reflect primarily the activity of the outer hair cells of the cochlea across a broad frequency range and are present in ears with hearing sensitivity better than 40-50 dB HL. In some embodiments, the methods provided herein result in an improved EOAEs response.

Immittance testing (tympanometry, acoustic reflex thresholds, acoustic reflex decay) assesses the peripheral auditory system, including middle ear pressure, tympanic membrane mobility, Eustachian tube function, and mobility of the middle ear ossicles. In some embodiments, the methods provided herein result in an improved immittance testing response.

The methods provided herein can result in an improved Distortion Product Otoacoustic Emissions (DPOAE) profile. For example, DPOAEs may be generated in the cochlea in response to two tones of a given frequency and sound pressure level presented in the ear canal. In certain embodiments, DPOAEs can serve as an objective indicator of normally functioning cochlea outer hair cells. According to some embodiments, the methods described herein results in preventing, delaying or slowing down the deterioration of DPOAE profile.

The methods provided herein can result in an improved speech comprehension. In some embodiments, method result can result in preventing, delaying or slowing down the deterioration of speech comprehension.

As used herein, a control level may be based on, for example, a level obtained from the subject, optionally, a sample from the subject, prior to administration of the rAAV. In some embodiments, the control level is based on a level resulting from the administration of a rAAV without the variant AAV capsid polypeptide, optionally, wherein the rAAV without the variant AAV capsid polypeptide comprises an rAAV capsid polypeptide selected from AAV2 and Anc80L65.

The methods provided herein can result in delivery to, and expression of a nucleic acid sequence encoding a gene of interest, such as GJB2, in a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, a border cell, an inner and/or outer cochlea hair cell, a spiral ganglion neuron, a vestibular hair cell, a vestibular support cell and/or a neuron of the vestibular ganglion. In certain embodiments, the method results in delivery to, and expression of, a nucleic acid sequence encoding a gene of interest, such as GJB2, in at least about 5%, 10%, 15%, 20%, 25%, 30%, 35%, 40%, 45%, 50%, 55%, 60%, 65%, 70%, 75%, 80%, 85%, 90%, 95%, or 99% of cells of the lateral wall or spiral ligament, support cells of the organ of Corti, fibrocytes of the spiral ligament, Claudius cells, Boettcher cells, cells of the spiral prominence, vestibular supporting cells, Hensen's cells, Deiters' cells, pillar cells, inner phalangeal cells, outer phalangeal cells, border cells, inner and outer cochlea hair cells, spiral ganglion neurons, vestibular hair cells, vestibular support cells and/or neurons of the vestibular ganglion.

### Pharmaceutical Compositions

According to some aspects, the disclosure provides pharmaceutical compositions comprising any of the AAV described herein, optionally in a pharmaceutically acceptable excipient. For example, the disclosure provides various compositions comprising an effective amount of a recombinant adeno-associated virus (rAAV) virion comprising: (i) a variant AAV capsid polypeptide which exhibits increased tropism in inner ear tissues or cells, optionally, as compared to a non-variant AAV capsid polypeptide; and (ii) a polynucleotide comprising a nucleic acid sequence encoding a gene of interest, such as GJB2.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for direct delivery to the ear (e.g., inner ear) which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991).

According to some embodiments, the pharmaceutical composition comprises one or more of BSST, PBS or BSS.

According to some embodiments, the pharmaceutical composition further comprises histidine buffer.

Although not required, the compositions may optionally be supplied in unit dosage form suitable for administration of a precise amount.

According to some embodiments, the compositions are administered to a subject prior to cochlear implant.

### Methods of Administration

Generally, the compositions described herein are formulated for administration to the ear. According to some embodiments, the compositions are formulated for administration to cells in the organ of Corti (OC) in the cochlea. Cells in the OC include hensen's cells, deiters' cells, pillar cells, inner phalangeal cells and/or outer phalangeal cells/border cells. The OC includes two classes of sensory hair cells: inner hair cells (IHCs), which convert mechanical information carried by sound into electrical signals transmitted to neuronal structures and outer hair cells (OHCs) which serve to amplify and tune the cochlear response, a process required for complex hearing function. According to some embodiments, the compositions are formulated for administration to the IHCs and/or the OHCs.

Injection to the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed through the oval or round window membrane. The round window membrane, which is a non-bony opening into the inner ear, is accessible in many animal models and administration of viral vector using this route is well tolerated. In humans, cochlear implant placement routinely relies on surgical electrode insertion through the round window membrane. According to some embodiments, the compositions are administered by injection via the round window membrane. According to some embodiments, the compositions are administered by injection into the scala tympani or scala media. According to some embodiments, the compositions are administered during a surgical procedure, e.g. during a cochleostomy or during a canalostomy.

According to some embodiments, the compositions are administered to the cochlea or vestibular system, optionally, wherein the delivery comprises direct administration into the cochlea or vestibular system via the round window membrane (RWM), oval window, or semi-circular canals. In some embodiments, the direct administration is by injection. In some embodiments, the administration is intravenous, intracerebroventricular, intracochlear, intrathecal, intramuscular, subcutaneous, or a combination thereof.

By safely and effectively transducing cochlear cells as described herein, the methods of the present disclosure may be used to treat an individual e.g., a human, wherein the transduced cells produce GJB2 in an amount sufficient to restore hearing or vestibular function for an extended period of time (e.g., months, years, decades, a lifetime)

According to the methods of treatment of the present disclosure, the volume of vector delivered may be determined based on the characteristics of the subject receiving the treatment, such as the age of the subject and the volume of the area to which the vector is to be delivered. According to some embodiments, the volume of the composition injected is between about 10 μl to about 1000 μl, or between about 10 μl and about 50 μl, or between about 25 μl and about 35 μl, or between about 100 μl to about 1000 μl, or between about between about 100 μl to about 500 μl, or between about 500 μl to about 1000 μl. According to some embodiments, the volume of the composition injected is more than about any one of 1 μl, 2 μl, 3 μl, 4 μl, 5 μl, 6 μl, 7 μl, 8 μl, 9 μl, 10 μl, 15 μl, 20 μl, 25 μl, 30 μl, 35 μl, 40 μl, 45 μl, 50 μl, 75 μl, 100 μl, 200 μl, 300 μl, 400 μl, 500 μl, 600 μl, 700 μl, 800 μl, 900 μl, or 1 mL, or any amount there between. According to some embodiments, the volume of the composition injected is at least about any one of 1 μl, 2 μl, 3 μl, 4 μl, 5 μl, 6 μl, 7 μl, 8 μl, 9 μl, 10 μl, 15 μl, 20 μl, 25 μl, 30 μl, 35 μl, 40 μl, 45 μl, 50 μl, 75 μl, 100 μl, 200 μl, 300 μl, 400 μl, 500 μl, 600 μl, 700 μl, 800 μl, 900 μl, or 1 mL, or any amount there between. According to some embodiments, the volume of the composition injected is about any one of 1 μl, 2 μl, 3 μl, 4 μl, 5 μl, 6 μl, 7 μl, 8 μl, 9 μl, 10 μl, 15 μl, 20 μl, 25 μl, 30 μl, 35 μl, 40 μl, 45 μl, 50 μl, 75 μl, 100 μl, 200 μl, 300 μl, 400 μl, 500 μl, 600 μl, 700 μl, 800 μl, 900 μl, or 1 mL, or any amount there between.

According to the methods of treatment of the present disclosure, the concentration of vector that is administered may differ depending on production method and may be chosen or optimized based on concentrations determined to be therapeutically effective for the particular route of administration. According to some embodiments, the concentration in vector genomes per milliliter (vg/ml) is selected from the group consisting of about 10⁸ vg/ml, about 10⁹ vg/ml, about 10¹⁰ vg/ml, about 10¹¹ vg/ml, about 10¹² vg/ml, about 10¹³ vg/ml, and about 10¹⁴ vg/ml. In preferred embodiments, the concentration is in the range of 10¹⁰ vg/ml-10¹³ vg/ml.

The effectiveness of the compositions described herein can be monitored by several criteria. For example, after treatment in a subject using methods of the present disclosure, the subject may be assessed for e.g., an improvement and/or stabilization and/or delay in the progression of one or more signs or symptoms of the disease state by one or more clinical parameters including those described herein. Examples of such tests are known in the art, and include objective as well as subjective (e.g., subject reported) measures. According to some embodiments, these tests may include, but are not limited to, auditory brainstem response (ABR) measurements, speech perception, mode of communication, and subjective assessments of aural response recognition.

According to some embodiments, subjects exhibiting nonsyndromic hearing loss and deafness (DFNB1) were first tested to determine their threshold hearing sensitivity over the auditory range. The subjects were then treated with the rAAV compositions described herein. Changes in the threshold hearing levels as a function of frequency measured in dB are determined. According to some embodiments, an improvement in hearing is determined as a 5 dB to 50 dB improvement in threshold hearing sensitivity in at least one ear at any frequency. According to some embodiments, an improvement in hearing is determined as a 10 dB to 30 dB improvement in threshold hearing sensitivity in at least one ear at any frequency. According to some embodiments, an improvement in hearing is determined as a 10 dB to 20 dB improvement in threshold hearing sensitivity in at least one ear any frequency.

### NON-LIMITING EMBODIMENTS

1. A method of treating or preventing hearing loss associated with deficiency of a gene, the method comprising administering to a subject in need thereof an effective amount of a recombinant adeno-associated virus (rAAV) virion comprising:
(i) a variant AAV capsid polypeptide which exhibits increased tropism in inner ear tissues or cells, optionally, as compared to a non-variant AAV capsid polypeptide; and
(ii) a polynucleotide comprising a nucleic acid sequence encoding the gene.
   2. A method of delivering a nucleic acid sequence encoding a gene associated with hearing loss to an inner ear tissue or cell comprising administering to a subject in need thereof an effective amount of a recombinant adeno-associated virus (rAAV) virion comprising:
(i) a variant AAV capsid polypeptide which exhibits increased tropism in inner ear tissues or cells, optionally, as compared to a non-variant AAV capsid polypeptide; and
(ii) a polynucleotide comprising a nucleic acid sequence encoding the gene.
   3. The method of Embodiment 1 or 2, wherein the inner ear tissues or cells are cochlear tissues or cells, or vestibular tissues or cells.
   4. The method of Embodiment 1 or 2, wherein the inner ear tissues or cells are cochlear tissues or cells.
   5. The method of any one of the preceding Embodiments, wherein the variant AAV capsid polypeptide is any variant AAV capsid polypeptide, optionally, selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; a variant AAV12 capsid polypeptide; and a variant Anc80 capsid polypeptide.
   6. The method of any one of the preceding Embodiments, wherein the variant AAV capsid polypeptide is a variant AAV2 capsid polypeptide.
   7. The method of any one of the preceding Embodiments, wherein:
(i) the variant AAV capsid polypeptide comprises an amino acid sequence listed in Table 1, or an amino acid sequence having at least about 85%, 90%, 95%, or 99% sequence identity thereto, optionally, wherein the AAV capsid is selected from the group consisting of a VP1, VP2, or VP3 capsid polypeptide; and/or
(ii) the variant AAV capsid polypeptide comprises an amino acid sequence listed in Table 1, or an amino acid sequence having at least about 85%, 90%, 95%, or 99% sequence homology thereto, optionally, wherein the AAV capsid is selected from the group consisting of a VP1, VP2, or VP3 capsid polypeptide.
   8. The method of any one of the preceding Embodiments, wherein the variant AAV capsid polypeptide comprises an amino acid sequence having one or more amino acid substitutions, insertions, and/or deletions relative to a wildtype AAV2 capsid polypeptide (SEQ ID NO: 18), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730.
   9. The method of Embodiment 8, wherein the variant AAV capsid polypeptide comprises an amino acid sequence having one or more amino acid substitutions relative to a wildtype AAV2 capsid polypeptide (SEQ ID NO: 18) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.
   10. The method of any one of the preceding Embodiments, wherein the variant AAV capsid polypeptide comprises:
(i) an amino acid sequence of any one of SEQ ID NOs: 27, 29, 31, 33, or 35;
(ii) an amino acid sequence having at least about 85%, 90%, 95%, or 99% sequence identity to any one of SEQ ID NOs: 27, 29, 31, 33, or 35; or
(iii) an amino acid sequence encoded by the nucleic acid sequence of any one of SEQ ID NOs: 26, 28, 30, 32, or 34.
   11. The method of any one of Embodiments 1-10, wherein the variant AAV capsid polypeptide comprises the amino acid sequence of SEQ ID NO: 27.
   12. The method of any one of Embodiments 1-10, wherein the variant AAV capsid polypeptide comprises the amino acid sequence of SEQ ID NO: 29.
   13. The method of any one of Embodiments 1-10, wherein the variant AAV capsid polypeptide comprises the amino acid sequence of SEQ ID NO: 31.
   14. The method of any one or Embodiments 1-10, wherein the variant AAV capsid polypeptide comprises the amino acid sequence of SEQ ID NO: 33.
   15. The method of any one of Embodiments 1-10, wherein the variant AAV capsid polypeptide comprises the amino acid sequence of SEQ ID NO: 35.
   16. The method of any one of the preceding Embodiments, wherein the variant AAV capsid polypeptide results in an increased level of rAAV tropism in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to a non-variant AAV capsid polypeptide.
   17. The method of any one of the preceding Embodiments, wherein the variant AAV capsid polypeptide results in an increased level of rAAV transduction efficiency in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to a non-variant AAV capsid polypeptide.
   18. The method of any one of the preceding Embodiments, wherein the method results in an increased expression of the gene in the inner ear tissues or cells, optionally, of at least about 1-fold, 1.25-fold, 1.5-fold, 1.75-fold, 2-fold, 2.5-fold, 3-fold, 4-fold, 5-fold, 6-fold, 7-fold, 8-fold, 9-fold, 10-fold, 12-fold, 14-fold, 16-fold, 18-fold, 20-fold, optionally, as compared to normal expression of the gene.
   19. The method of any one of the preceding Embodiments, wherein the method results in an overexpression of GJB2 expression in the inner ear tissues or cells.
   20. The method of any one of the preceding Embodiments, wherein the method results in a decreased level of rAAV neutralizing antibody (NAb) titers, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a control level.
   21. The method of any one of the preceding Embodiments, wherein the method results in a decreased level of inner ear inflammation or toxicity, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of inner ear inflammation or toxicity prior to administration.
   22. The method of any one of the preceding Embodiments, wherein the method results in a delay in progression of inner ear inflammation or toxicity, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to progression of inner ear inflammation or toxicity prior to administration.
   23. The method of any one of the preceding Embodiments, wherein the method results in a decreased level of hair cell loss, degeneration, and/or death, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of hair cell loss, degeneration, and/or death prior to administration.
   24. The method of any one of the preceding Embodiments, wherein the method results in a decreased level of spiral ganglion neuron loss, degeneration, and/or death, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of spiral ganglion neuron loss, degeneration, and/or death prior to administration.
   25. The method of any one of the preceding Embodiments, wherein the method results in an decreased auditory brainstem response (ABR) threshold at any frequency, optionally, of at least about 5%, 10%, 15%, 20%, 25%, 30%, 35%, 40%, 45%, 50%, 55%, 60%, 65%, 70%, 75%, 80%, 85%, 90%, 95%, or 99%, optionally, as compared to a level of ABR threshold prior to administration.
   26. The method of any one of the preceding Embodiments, wherein the method results in an improved Distortion Product Otoacoustic Emissions (DPOAE) profile.
   27. The method of any one of the preceding Embodiments, wherein the method results in preventing, delaying or slowing down the deterioration of DPOAE profile.
   28. The method of any one of the preceding Embodiments, wherein the method results in an improved speech comprehension and/or speech intelligibility.
   29. The method of any one of the preceding Embodiments, wherein the method results in preventing, delaying or slowing down the deterioration of speech comprehension and/or speech intelligibility.
   30. The method of any one of Embodiments 16-29, wherein the control level is based on: a level obtained from the subject, optionally, a sample from the subject, prior to administration of the rAAV.
   31. The method of any one of Embodiments 16-29, wherein the control level is based on: a level resulting from the administration of a rAAV without the variant AAV capsid polypeptide, optionally, wherein the rAAV without the variant AAV capsid polypeptide comprises an rAAV capsid polypeptide selected from AAV2 and Anc80L65.
   32. The method of any one of the preceding Embodiments, wherein the method results in delivery to, and expression of a nucleic acid sequence encoding GJB2 in, a cell of the lateral wall or spiral ligament, a support cell of the organ of Corti, a fibrocyte of the spiral ligament, a Claudius cell, a Boettcher cell, a cell of the spiral prominence, a vestibular supporting cell, a Hensen's cell, a Deiters' cell, a pillar cell, an inner phalangeal cell, an outer phalangeal cell, and/or a border cell.
   33. The method of any one of the preceding Embodiments, wherein the method results in delivery to, and expression of, a nucleic acid sequence encoding GJB2 in at least about 5%, 10%, 15%, 20%, 25%, 30%, 35%, 40%, 45%, 50%, 55%, 60%, 65%, 70%, 75%, 80%, 85%, 90%, 95%, or 99% of cells of the lateral wall or spiral ligament, support cells of the organ of Corti, fibrocytes of the spiral ligament, Claudius cells, Boettcher cells, cells of the spiral prominence, vestibular supporting cells, Hensen's cells, Deiters' cells, pillar cells, inner phalangeal cells, outer phalangeal cells, border cells, inner cochlear hair cells, outer cochlear hair cells, spiral ganglion neurons, vestibular hair cells, vestibular support cells, and/or vestibular ganglion neurons.
   34. The method of any one of the preceding Embodiments, wherein the gene is GJB2.
   35. The method of any one of the preceding Embodiments, wherein the nucleic acid sequence encoding GJB2 is a non-naturally occurring sequence.
   36. The method of any one of the preceding Embodiments, wherein the nucleic acid sequence encoding GJB2 encodes mammalian GJB2.
   37. The method of any one of the preceding Embodiments, wherein the nucleic acid sequence encoding GJB2 encodes human, mouse, non-human primate, or rat GJB2.
   38. The method of any one of the preceding Embodiments, wherein the nucleic acid sequence encoding GJB2 comprises SEQ ID NO: 10.
   39. The method of any one of the preceding Embodiments, wherein the nucleic acid sequence encoding GJB2 is codon optimized for mammalian expression.
   40. The method of Embodiment 39, wherein the nucleic acid sequence encoding GJB2 comprises SEQ ID NO: 11, SEQ ID NO: 12 or SEQ ID NO: 13.
   41. The method of Embodiment 40, wherein the nucleic acid sequence encoding GJB2 is codon optimized for expression in human, rat, non-human primate, guinea pig, mini pig, pig, cat, sheep, or mouse cells.
   42. The method of any one of the preceding Embodiments, wherein the nucleic acid sequence encoding GJB2 is a cDNA sequence.
   43. The method of any one of the preceding Embodiments, wherein the nucleic acid sequence encoding GJB2 further comprises an operably linked C-terminal tag or N-terminal tag.
   44. The method of Embodiment 43, wherein the tag is a FLAG-tag or a HA-tag.
   45. The method of any one of the preceding Embodiments, wherein the nucleic acid sequence encoding GJB2 is operably linked to a promoter.
   46. The method of Embodiment 45, wherein the promoter is an ubiquitously-active CBA, small CBA (smCBA), EF1a, CASI promoter, a cochlear-support cell promoter, GJB2 expression-specific GFAP promoter, small GJB2 promoter, medium GJB2 promoter, large GJB2 promoter, a sequential combination of 2-3 individual GJB2 expression-specific promoters, or a synthetic promoter.
   47. The method of Embodiment 45 or 46, wherein the promoter is optimized to drive sufficient GJB2 expression to treat or prevent hearing loss.
   48. The method of any one of the preceding Embodiments, wherein the nucleic acid sequence encoding GJB2 further comprises an operably linked 3′UTR regulatory region.
   49. The method of any one of the preceding Embodiments, wherein the nucleic acid sequence encoding GJB2 further comprises an operably linked 3′UTR regulatory region comprising a Woodchuck Hepatitis Virus Postranscriptional Regulatory Element (WPRE).
   50. The method of any one of the preceding Embodiments, wherein the nucleic acid sequence encoding GJB2 further comprises an operably linked polyadenylation signal.
   51. The method of Embodiment 50, wherein the polyadenylation signal is an SV40 polyadenylation signal.
   52. The method of Embodiment 50, wherein the polyadenylation signal is a human growth hormone (hGH) polyadenylation signal.
   53. The method of any one of the preceding Embodiments, wherein the polynucleotide further comprises a 27-nucleotide hemagglutinin C-terminal tag or a 24-nucleotide Flag tag; operably linked to one of the following promoter elements optimized to drive high GJB2 expression: (a) an ubiquitously-active CBA, small CBA (smCBA), EF1a, or CASI promoter; (b) a cochlear-support cell or GJB2 expression-specific 1.68 kb GFAP, small/medium/large GJB2 promoters, a sequential combination of 2-3 individual GJB2 expression-specific promoters, or a synthetic promoter; operably linked to a 3′-UTR regulatory region comprising the Woodchuck Hepatitis Virus Posttranscriptional Regulatory Element (WPRE) followed by either a SV40 or human growth hormone (hGH) polyadenylation signal.
   54. The method of any one of the preceding Embodiments, wherein the polynucleotide further comprises an AAV genomic cassette, optionally, wherein:
(i) the AAV genomic cassette is flanked by two sequence-modulated inverted terminal repeats, preferably about 143-bases in length; or
(ii) the AAV genomic cassette is flanked by a self-complimentary AAV (scAAV) genomic cassette consisting of two inverted identical repeats, preferably no longer than 2.4 kb, separated by an about 113-bases scAAV-enabling ITR (ITRΔtrs) and flanked on either end by about 143-bases sequence-modulated ITRs.
   55. The method of any one of the preceding Embodiments, wherein the polynucleotide comprises a codon/sequence-optimized human GJB2 cDNA with or without a hemagglutinin C-terminal tag or a Flag tag, preferably about 27-nucleotide in length, optionally about a 0.68 kilobase (kb) in size; operably linked to one of the following promoter elements optimized to drive high GJB2 expression: (a) an ubiquitously-active CBA, preferably about 1.7 kb in size, small CBA (smCBA), preferably about 0.96 kb in size, EF1a, preferably about 0.81 kb in size, or CASI promoter, preferably about 1.06 kb in size; (b) a cochlear-support cell or GJB2 expression-specific GFAP promoter, preferably about 1.68 kb in size, small GJB2 promoter, preferably about 0.13 kb in size, medium GJB2 promoter, preferably about 0.54 kb in size, large GJB2 promoter, preferably about 1.0 kb in size, or a sequential combination of 2-3 individual GJB2 expression-specific promoters; operably linked to a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, and further comprising either two about 143-base sequence-modulated inverted terminal repeats (ITRs) flanking the AAV genomic cassette or a self-complimentary AAV (scAAV) genomic cassette consisting of two inverted identical repeats, preferably no longer than 2.4 kb, separated by an about 113-base scAAV-enabling ITR (ITRΔtrs) and flanked on either end by about 143-base sequence-modulated ITRs.
   56. The method of any one of the preceding Embodiments, wherein the hearing loss is genetic hearing loss.
   57. The method of any one of the preceding Embodiments, wherein the hearing loss is DFNB1 hearing loss.
   58. The method of any one of the preceding Embodiments, wherein the hearing loss is caused by a mutation in GJB2, optionally, wherein the mutation is a homozygous mutation or a heterozygous mutation.
   59. The method of any one of the preceding Embodiments, wherein the hearing loss is caused by an autosomal recessive GJB2 mutants (DFNB1).
   60. The method of any one of the preceding Embodiments, wherein the hearing loss is caused by an autosomal dominant GJB2 mutants (DFNA3A).
   61. The method of any one of the preceding Embodiments, wherein the administration is to the cochlea or vestibular system, optionally, wherein the delivery comprises direct administration into the cochlea or vestibular system via the round window membrane (RWM), oval window, or semi-circular canals.
   62. The method of Embodiment 61, wherein the direct administration is injection.
   63. The method of any one of Embodiments 1-60, wherein the administration is intravenous, intracerebroventricular, intracochlear, intrathecal, intramuscular, subcutaneous, or a combination thereof.
   64. A composition for use in treating or preventing hearing loss associated with deficiency of a gene in a subject in need thereof, wherein the composition comprises a recombinant adeno-associated virus (rAAV) virion comprising:
(i) a variant AAV capsid polypeptide which exhibits increased tropism in inner ear tissues or cells, optionally, as compared to a non-variant AAV capsid polypeptide; and
(ii) a polynucleotide comprising a nucleic acid sequence encoding the gene.
   65. A composition for delivering a nucleic acid sequence encoding a gene associated with hearing loss to an inner ear tissue or cell of a subject in need thereof, wherein the composition comprises an effective amount of a recombinant adeno-associated virus (rAAV) virion comprising:
(i) a variant AAV capsid polypeptide which exhibits increased tropism in inner ear tissues or cells, optionally, as compared to a non-variant AAV capsid polypeptide; and
(ii) a polynucleotide comprising a nucleic acid sequence encoding the gene.
   66. The composition of Embodiment 64 or 65, wherein the inner ear tissues or cells are cochlear tissues or cells, or vestibular tissues or cells.
   67. The composition of Embodiment 64 or 65, wherein the inner ear tissues or cells are cochlear tissues or cells.
   68. The composition of any one of Embodiments 64-67, wherein the variant AAV capsid polypeptide is selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; and a variant AAV12 capsid polypeptide.
   69. The composition of any one of Embodiments 64-68, wherein the variant AAV capsid polypeptide is a variant AAV2 capsid polypeptide.
   70. The composition of any one of Embodiments 64-69, wherein the variant AAV capsid polypeptide comprises an amino acid sequence listed in Table 1, or an amino acid sequence having at least about 85%, 90%, 95%, or 99% sequence identity thereto, optionally, wherein the AAV capsid is selected from the group consisting of a VP1, VP2, or VP3 capsid polypeptide.
   71. The composition of any one of Embodiments 64-70, wherein the variant AAV capsid polypeptide comprises an amino acid sequence having one or more amino acid substitutions, insertions, and/or deletions relative to a wildtype AAV2 capsid polypeptide (SEQ ID NO: 1), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730.
   72. The composition of Embodiment 71, wherein the variant AAV capsid polypeptide comprises an amino acid sequence having one or more amino acid substitutions relative to a wildtype AAV2 capsid polypeptide (SEQ ID NO: 1) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.
   73. The composition of any one of Embodiments 64-72, wherein the variant AAV capsid polypeptide comprises:
(i) an amino acid sequence of any one of SEQ ID NOs: 27, 29, 31, 33, or 35;
(ii) an amino acid sequence having at least about 85%, 90%, 95%, or 99% sequence identity to any one of SEQ ID NOs: 27, 29, 31, 33, or 35; or
(iii) an amino acid sequence encoded by the nucleic acid sequence of any one of SEQ ID NOs: 26, 28, 30, 32, or 34.
   74. The composition of any one of Embodiments 64-73, wherein the variant AAV capsid polypeptide comprises the amino acid sequence of SEQ ID NO: 27.
   75. The composition of any one of Embodiments 64-73, wherein the variant AAV capsid polypeptide comprises the amino acid sequence of SEQ ID NO: 29.
   76. The composition of any one of Embodiments 64-73, wherein the variant AAV capsid polypeptide comprises the amino acid sequence of SEQ ID NO: 31.
   77. The composition of any one of Embodiments 64-73, wherein the variant AAV capsid polypeptide comprises the amino acid sequence of SEQ ID NO: 33.
   78. The composition of any one of Embodiments 64-73, wherein the variant AAV capsid polypeptide comprises the amino acid sequence of SEQ ID NO: 35.
   79. The composition of any one of Embodiments 64-78, wherein the gene is GJB2.
   80. The composition of any one of Embodiments 64-79, wherein the nucleic acid sequence encoding GJB2 is a non-naturally occurring sequence.
   81. The composition of any one of Embodiments 64-80, wherein the nucleic acid sequence encoding GJB2 encodes mammalian GJB2.
   82. The composition of any one of Embodiments 64-81, wherein the nucleic acid sequence encoding GJB2 encodes human, mouse, non-human primate, mini pig, or rat GJB2.
   83. The composition of any one of Embodiments 64-82, wherein the nucleic acid sequence encoding GJB2 comprises SEQ ID NO: 10.
   84. The composition of any one of Embodiments 64-83, wherein the nucleic acid sequence encoding GJB2 is codon optimized for mammalian expression.
   85. The composition of Embodiment 84, wherein the nucleic acid sequence encoding GJB2 comprises SEQ ID NO: 11, SEQ ID NO: 12 or SEQ ID NO: 13.
   86. The composition of Embodiment 84, wherein the nucleic acid sequence encoding GJB2 is codon optimized for expression in human, rat, non-human primate, guinea pig, mini pig, pig, cat, sheep, or mouse cells.
   87. The composition of any one of Embodiments 64-86, wherein the nucleic acid sequence encoding GJB2 is a cDNA sequence.
   88. The composition of any one of Embodiments 64-87, wherein the nucleic acid sequence encoding GJB2 further comprises an operably linked C-terminal tag or N-terminal tag.
   89. The composition of Embodiment 88, wherein the tag is a FLAG-tag or a HA-tag.
   90. The composition of any one of Embodiments 64-89, wherein the nucleic acid sequence encoding GJB2 is operably linked to a promoter.
   91. The composition of Embodiment 90, wherein the promoter is an ubiquitously-active CBA, small CBA (smCBA), EF1a, CASI promoter, a cochlear-support cell promoter, GJB2 expression-specific GFAP promoter, small GJB2 promoter, medium GJB2 promoter, large GJB2 promoter, a sequential combination of 2-3 individual GJB2 expression-specific promoters, or a synthetic promoter.
   92. The composition of Embodiment 90 or 91, wherein the promoter is optimized to drive sufficient GJB2 expression to treat or prevent hearing loss.
   93. The composition of any one of Embodiments 64-92, wherein the nucleic acid sequence encoding GJB2 further comprises an operably linked 3′UTR regulatory region.
   94. The composition of any one of Embodiments 64-93, wherein the nucleic acid sequence encoding GJB2 further comprises an operably linked 3′UTR regulatory region comprising a Woodchuck Hepatitis Virus Postranscriptional Regulatory Element (WPRE).
   95. The composition of any one of Embodiments 64-94, wherein the nucleic acid sequence encoding GJB2 further comprises an operably linked polyadenylation signal.
   96. The composition of Embodiment 95, wherein the polyadenylation signal is an SV40 polyadenylation signal.
   97. The composition of Embodiment 95, wherein the polyadenylation signal is a human growth hormone (hGH) polyadenylation signal.
   98. The composition of any one of Embodiments 64-97, wherein the polynucleotide further comprising a 27-nucleotide hemagglutinin C-terminal tag or a 24-nucleotide Flag tag; operably linked to one of the following promoter elements optimized to drive high GJB2 expression: (a) an ubiquitously-active CBA, small CBA (smCBA), EF1a, or CASI promoter; (b) a cochlear-support cell or GJB2 expression-specific 1.68 kb GFAP, small/medium/large GJB2 promoters, a sequential combination of 2-3 individual GJB2 expression-specific promoters, or a synthetic promoter; operably linked to a 3′-UTR regulatory region comprising the Woodchuck Hepatitis Virus Posttranscriptional Regulatory Element (WPRE) followed by either a SV40 or human growth hormone (hGH) polyadenylation signal.
   99. The composition of any one of Embodiments 64-98, wherein the polynucleotide further comprises an AAV genomic cassette, optionally, wherein:
(i) the AAV genomic cassette is flanked by two sequence-modulated inverted terminal repeats, preferably about 143-bases in length; or
(ii) the AAV genomic cassette is flanked by a self-complimentary AAV (scAAV) genomic cassette consisting of two inverted identical repeats, preferably no longer than 2.4 kb, separated by an about 113-bases scAAV-enabling ITR (ITRΔtrs) and flanked on either end by about 143-bases sequence-modulated ITRs.
   100. The composition of any one of Embodiments 64-99, wherein the polynucleotide comprises a codon/sequence-optimized human GJB2 cDNA with or without a hemagglutinin C-terminal tag or a Flag tag, preferably about 27-nucleotide in length, optionally about a 0.68 kilobase (kb) in size; operably linked to one of the following promoter elements optimized to drive high GJB2 expression: (a) an ubiquitously-active CBA, preferably about 1.7 kb in size, small CBA (smCBA), preferably about 0.96 kb in size, EF1a, preferably about 0.81 kb in size, or CASI promoter, preferably about 1.06 kb in size; (b) a cochlear-support cell or GJB2 expression-specific GFAP promoter, preferably about 1.68 kb in size, small GJB2 promoter, preferably about 0.13 kb in size, medium GJB2 promoter, preferably about 0.54 kb in size, large GJB2 promoter, preferably about 1.0 kb in size, or a sequential combination of 2-3 individual GJB2 expression-specific promoters; operably linked to a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, and further comprising either two about 143-base sequence-modulated inverted terminal repeats (ITRs) flanking the AAV genomic cassette or a self-complimentary AAV (scAAV) genomic cassette consisting of two inverted identical repeats, preferably no longer than 2.4 kb, separated by an about 113-base scAAV-enabling ITR (ITRΔtrs) and flanked on either end by about 143-base sequence-modulated ITRs.
   101. A method of treating or preventing hearing loss comprising administering to a subject in need thereof an effective amount of a composition of any one of Embodiments 64-100.
   102. A method of delivering a nucleic acid sequence encoding a gene associated with hearing loss to an inner ear tissue or cell comprising administering to a subject in need thereof an effective amount of a composition of any one of Embodiments 64-100.
   103. A method of delivering a nucleic acid sequence encoding GJB2 to an inner ear tissue or cell comprising administering to a subject in need thereof an effective amount of a composition of any one of Embodiments 64-100.
   104. The method of composition of any one of previous Embodiments, wherein the subject is a mammal.
   105. The method of composition of any one of previous Embodiments, wherein the subject is a ptimate.

Further embodiments of the present disclosure will now be described with reference to the following examples. The examples contained herein are offered by way of illustration and not by any way of limitation.

### NON-LIMITING EXAMPLES

### Example 1. Characterization of Novel AAV Capsid Variants for Delivery of GJB2 Gene Therapy for Congenital Hearing Loss

To identify optimal capsids for gene therapy, novel and previously described AAV capsid variants were evaluated for expression ex vivo in rat and mouse inner ear tissues and in vivo in non-human primates (NHP). Exemplary AAV capsid sequences are provided in Table 1.

TABLE 1
Amino Acid and Nucleic Acid Sequences of Exemplary AAV
Capsid Polypepties

| | SEQ | |
|---|---|---|
| | ID | |
| Capsid Name | NO: | Sequence |
| wtAAV2 | 18 | ATGGCTGCCGATGGTTATCTTCCAGATTGGCTCGAGGACA |
| reference | | CTCTCTCTGAAGGAATAAGACAGTGGTGGAAGCTCAAACC |
| (nucleotide) | | TGGCCCACCACCACCAAAGCCCGCAGAGCGGCATAAGGA |
| | | CGACAGCAGGGGTCTTGTGCTTCCTGGGTACAAGTACCTC |
| | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGCGAGTATCAAAGACATC |
| | | TGCGGATAACAACAACAGTGAATACTCGTGGACTGGAGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| wtAAV2 | 19 | MAADGYLPDWLEDTLSEGIRQWWKLKPGPPPPKPAERHKD |
| reference | | DSRGLVLPGYKYLGPFNGLDKGEPVNEADAAALEHDKAYD |
| (amino acid) | | RQLDSGDNPYLKYNHADAEFQERLKEDTSFGGNLGRAVFQA |
| | | KKRVLEPLGLVEEPVKTAPGKKRPVEHSPVEPDSSSGTGKAG |
| | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTSA |
| | | DNNNSEYSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0010 | 20 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also refered to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-903 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTTCTTAAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGCGAGTATCAAAGACATC |
| | | TGCGGATAACAACAACAGTGAATTCTCGTGGACCGGTGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGTACCAGATTCCT |
| | | GACTCGTAATCTGTAA |
| R-AGTC-0010 | 21 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also refered to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-903 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYFLSRTNTPS |
| | | GTTTQSRLQFSQAGASDIRDQSRNWLPGPCYRQQRVSKTSA |
| | | DNNNSEFSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRFLTRNL |
| R-AGTC-0018 | 22 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-906 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCAACGCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGCGAGTATCAAAGACAGA |
| | | TGGAGAAAACAACAACAGTGATTTCTCGTGGACTGGAGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | AGCGCAGCGGGAGCAGATGTGGCAATTGATAGTGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0018 | 23 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-906 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSNAGASNDNHYFGYSTPWGYFDEN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTDG |
| | | ENNNSDFSWTGATKYHLNGRDSLVNPGPAMASHKDDEEKF |
| | | FPQSGVLIFGKQSAAGADVAIDSVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0030 | 24 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| here as: | | CGACAGCAGGGGTCTTGTGCTTCCTGGGTACAAGTACCTC |
| OMY-907 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCAACGCA |
| | | GGAGCCAGCAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTTCTTGAGCAGAACAAACACCGCGAG |
| | | CGGAAACGTCACGCAGTCAACGCTTCAGTTTTCTCAGGCC |
| | | GGAGCGAGTGACATTCGGGACCAGTCTAGGAACTGGCTTC |
| | | CTGGACCCTGTTACCGCCAGCAGCGAGTATCAAAGACATC |
| | | TGCGGATAACAACAACAGTGAATACTCGTGGACTGGAGCT |
| | | ACCAAGTACCACCTCAATGGCAGAGACTCTCTGGTGAATC |
| | | CGGGCCCGGCCATGGCAAGCCACAGGGACGATGACGACA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0030 | 25 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| here as: | | KKRVLEPLGLVEEPVKTAPGKKRPVEHSPVEPDSSSGTGKAG |
| OMY-907 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSNAGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYFLSRTNTAS |
| | | GNVTQSTLQFSQAGASDIRDQSRNWLPGPCYRQQRVSKTSA |
| | | DNNNSEYSWTGATKYHLNGRDSLVNPGPAMASHRDDDDKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0053 | 26 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-911 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGAGAGTCTCAAAAACAGA |
| | | CGGCGAGAACAACAACAGTGACTTCTCCTGGACAGGAGCT |
| | | ACCAAGTACCACCTCAATGGCAGAGACTCTCTGGTGAATC |
| | | CCGGACCAGCTATGGCAAGCCACAAGGACGATGAAGAAA |
| | | AGTTTTTTCCTCAGAGCGGGGTTCTCATCTTTGGGAAGGA |
| | | AGACGCCACCGAAAACAATATCGACATCGACCGGGTCAT |
| | | GATTACAGACGAAGAGGAAATCAGGACAACCAATCCCGT |
| | | GGCTACGGAGCAGTATGGTTCTGTATCTACCAACCTCCAG |
| | | AGAGGCAACAGACAAGCAGCTACCGCAGATGTCAACACA |
| | | CAAGGCGTTCTTCCAGGCATGGTCTGGCAGGACAGAGATG |
| | | TGTACCTTCAGGGGCCCATCTGGGCAAAGATTCCACACAC |
| | | GGACGGACATTTTCACCCCTCTCCCCTCATGGGTGGATTCG |
| | | GACTTAAACACCCTCCTCCACAGATTCTCATCAAGAACAC |
| | | CCCGGTACCTGCGAATCCTTCGACCACCTTCAGTGCGGCA |
| | | AAGTTTGCTTCCTTCATCACACAGTACTCCACGGGACAGG |
| | | TCAGCGTGGAGATCGAGTGGGAGCTGCAGAAGGAAAACA |
| | | GCAAACGCTGGAATCCCGAAATTCAGTACACTTCCAACTA |
| | | CAACAAGTCTGTTAATGTGGACTTTACTGTGGACACTAAT |
| | | GGCGTGTATTCAGAGCCTCGCCCCATTGGCACCAGATACC |
| | | TGACTCGTAATCTGTAA |
| R-AGTC-0053 | 27 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-911 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTDG |
| | | ENNNSDFSWTGATKYHLNGRDSLVNPGPAMASHKDDEEKF |
| | | FPQSGVLIFGKEDATENNIDIDRVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0054 | 28 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| hereing as: | | CGACAGCAGGGGTCTTGTGCTTCCTGGGTACAAGTACCTC |
| OMY-912 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGCGAGTATCAAAGACATC |
| | | TGCGGATAACAACAACAGTGAATACTCGTGGACTGGAGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0054 | 29 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| hereing as: | | KKRVLEPLGLVEEPVKTAPGKKRPVEHSPVEPDSSSGTGKAG |
| OMY-912 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTSA |
| | | DNNNSEYSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0055 | 30 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-913 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCAACGCA |
| | | GGAGCCAGCAACGACAATCACTTCTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGATGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTTCTTAAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGAGAGTCTCAAAAGTCGA |
| | | CGGCGAGAACAACAACAGTGACTTCTCCTGGACAGGAGCT |
| | | ACCAAGTACCACCTCAATGGCAGAGACTCTCTGGTGAATC |
| | | CGGGACCGGCCATGGCAAGCCACAAGGACGATGAAGAAA |
| | | AGTTTTTTCCTCAGAGCGGGGTTCTCATCTTTGGGAAGCAA |
| | | AGCGCCGCCGGAGCCGATGTCGCGATCGACAGCGTCATGA |
| | | TTACAGACGAAGAGGAAATCAGGACAACCAATCCCGTGG |
| | | CTACGGAGCAGTATGGTTCTGTATCTACCAACCTCCAGAG |
| | | AGGCAACAGACAAGCAGCTACCGCAGATGTCAACACACA |
| | | AGGCGTTCTTCCAGGCATGGTCTGGCAGGACAGAGATGTG |
| | | TACCTTCAGGGGCCCATCTGGGCAAAGATTCCACACACGG |
| | | ACGGACATTTTCACCCCTCTCCCCTCATGGGTGGATTCGGA |
| | | CTTAAACACCCTCCTCCACAGATTCTCATCAAGAACACCC |
| | | CGGTACCTGCGAATCCTTCGACCACCTTCAGTGCGGCAAA |
| | | GTTTGCTTCCTTCATCACACAGTACTCCACGGGACAGGTC |
| | | AGCGTGGAGATCGAGTGGGAGCTGCAGAAGGAAAACAGC |
| | | AAACGCTGGAATCCCGAAATTCAGTACACTTCCAACTACA |
| | | ACAAGTCTGTTAATGTGGACTTTACTGTGGACACTAATGG |
| | | CGTGTATTCAGAGCCTCGCCCCATTGGTACCAGATTCCTG |
| | | ACTCGTAATCTGTAA |
| R-AGTC-0055 | 31 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-913 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSNAGASNDNHFFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYFLSRTNTPS |
| | | GTTTQSRLQFSQAGASDIRDQSRNWLPGPCYRQQRVSKVDG |
| | | ENNNSDFSWTGATKYHLNGRDSLVNPGPAMASHKDDEEKF |
| | | FPQSGVLIFGKQSAAGADVAIDSVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRFLTRNL |
| R-AGTC-0056 | 32 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-914 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCGCATCA |
| | | GGAGCCAGCAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGAGAGTCTCAAAACAAGA |
| | | CGCCGAGAACAACAACAGTGAGTTCTCCTGGCCAGGAGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAATAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0056 | 33 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-914 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSASGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKQDA |
| | | ENNNSEFSWPGATKYHLNGRDSLVNPGPAMASHKDDEEKFF |
| | | PQSGVLIFGKQGSEKTNVDIEKVMITDEEEIRTTNPVATEQYG |
| | | SVSTNLQRGNRQAATADVNTQGVLPGMVWQDRDVYLQGPI |
| | | WAKIPHTDGHFHPSPLMGGFGLKHPPPQILIKNTPVPANPSTT |
| | | FSAAKFASFITQYSTGQVSVEIEWELQKENSKRWNPEIQYTSN |
| | | YNKSVNVDFTVDTNGVYSEPRPIGTRYLTRNL |
| R-AGTC-0057 | 34 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| hereing as: | | CGACAGCAGGGGTCTTGTGCTTCCTGGGTACAAGTACCTC |
| OMY-915 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGACGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTTCTTGAGCAGAACAAACACCGGGGC |
| | | CGGAAACATGACGACCTCAGCGCTTAGGTTTTCTCAGGCC |
| | | GGAGCGAGTGACATTCGGGACCAGTCTAGGAACTGGCTTC |
| | | CTGGACCCTGTTACCGCCAGCAGCGAGTATCAACAACACC |
| | | CGCCGACAACAACAACAGTGACTTCTCGTGGACTGGAGCT |
| | | ACCAAGTACCACCTCAATGGCAGAGACTCTCTGGTGAATC |
| | | CGGGCCCGGCCATGGCAAGCCACAAGGACGATGACGAGA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAAATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0057 | 35 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| hereing as: | | KKRVLEPLGLVEEPVKTAPGKKRPVEHSPVEPDSSSGTGKAG |
| OMY-915 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYFLSRTNTGA |
| | | GNMTTSALRFSQAGASDIRDQSRNWLPGPCYRQQRVSTTPA |
| | | DNNNSDFSWTGATKYHLNGRDSLVNPGPAMASHKDDDEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0058 | 36 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| hereing as: | | CGACAGCAGGGGTCTTGTGCTTCCTGGGTACAAGTACCTC |
| OMY-916 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCCAATCA |
| | | GGAGCCTCGAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGATGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTACTTGAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGAGAGTCTCAAAAACAGA |
| | | CGGCGAGAACAACAACAGTGACTTCTCCTGGACAGGAGCT |
| | | ACCAAGTACCACCTCAATGGCAGAGACTCTCTGGTGAATC |
| | | CGGGCCCGGCCATGGCAAGCCACAAGGACGATGAAGAAA |
| | | AGTTTTTTCCTCAGAGCGGGGTTCTCATCTTTGGGAAGCAA |
| | | GGCTCAGAGAAAACAAATGTGGACATTGAAAAGGTCATG |
| | | ATTACAGACGAAGAGGAGATCAGGACAACCAATCCCGTG |
| | | GCTACGGAGCAGTATGGTTCTGTATCTACCAACCTCCAGA |
| | | GAGGCAACAGACAAGCAGCTACCGCAGATGTCAACACAC |
| | | AAGGCGTTCTTCCAGGCATGGTCTGGCAGGACAGAGATGT |
| | | GTACCTTCAGGGGCCCATCTGGGCAAAGATTCCACACACG |
| | | GACGGACATTTTCACCCCTCTCCCCTCATGGGTGGATTCGG |
| | | ACTTAAACACCCTCCTCCACAGATTCTCATCAAGAACACC |
| | | CCGGTACCTGCGAATCCTTCGACCACCTTCAGTGCGGCAA |
| | | AGTTTGCTTCCTTCATCACACAGTACTCCACGGGACAGGT |
| | | CAGCGTGGAGATCGAGTGGGAGCTGCAGAAGGAAAACAG |
| | | CAAACGCTGGAATCCCGAAATTCAGTACACTTCCAACTAC |
| | | AACAAGTCTGTTAATGTGGACTTTACTGTGGACACTAATG |
| | | GCGTGTATTCAGAGCCTCGCCCCATTGGCACCAGATACCT |
| | | GACTCGTAATCTGTAA |
| R-AGTC-0058 | 37 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| hereing as: | | KKRVLEPLGLVEEPVKTAPGKKRPVEHSPVEPDSSSGTGKAG |
| OMY-916 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTDG |
| | | ENNNSDFSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| R-AGTC-0059 | 38 | ATGGCTGCCGATGGTTATCTTCCAGATTGGCTCGAGGACA |
| (nucleotide) | | CTCTCTCTGAAGGAATAAGACAGTGGTGGAAGCTCAAACC |
| Also referred to | | TGGCCCACCACCACCAAAGCCCGCAGAGCGGCATAAGGA |
| herein as: | | CGACAGCAGGGGTCTTGTGCTTCCTGGGTACAAGTACCTC |
| OMY-917 | | GGACCCTTCAACGGACTCGACAAGGGAGAGCCGGTCAAC |
| | | GAGGCAGACGCCGCGGCCCTCGAGCACGACAAAGCCTAC |
| | | GACCGGCAGCTCGACAGCGGAGACAACCCGTACCTCAAG |
| | | TACAACCACGCCGACGCGGAGTTTCAGGAGCGCCTTAAAG |
| | | AAGATACGTCTTTTGGGGGCAACCTCGGACGAGCAGTCTT |
| | | CCAGGCGAAAAAGAGGGTTCTTGAACCTCTGGGCCTGGTT |
| | | GAGGAACCTGTTAAGACGGCTCCGGGAAAAAAGAGGCCG |
| | | GTAGAGCACTCTCCTGTGGAGCCAGACTCCTCCTCGGGAA |
| | | CCGGAAAGGCGGGCCAGCAGCCTGCAAGAAAAAGATTGA |
| | | ATTTTGGTCAGACTGGAGACGCAGACTCAGTACCTGACCC |
| | | CCAGCCTCTCGGACAGCCACCAGCAGCCCCCTCTGGTCTG |
| | | GGAACTAATACGATGGCTACAGGCAGTGGCGCACCAATG |
| | | GCAGACAATAACGAGGGCGCCGACGGAGTGGGTAATTCC |
| | | TCGGGAAATTGGCATTGCGATTCCACATGGATGGGCGACA |
| | | GAGTCATCACCACCAGCACCCGAACCTGGGCCCTGCCCAC |
| | | CTACAACAACCACCTCTACAAACAAATTTCCAGCAACGCA |
| | | GGAGCCAGCAACGACAATCACTACTTTGGCTACAGCACCC |
| | | CTTGGGGGTATTTTGACTTCAACAGATTCCACTGCCACTTT |
| | | TCACCACGTGACTGGCAAAGACTCATCAACAACAACTGGG |
| | | GATTCCGACCCAAGAGACTCAACTTCAAGCTCTTTAACAT |
| | | TCAAGTCAAAGAGGTCACGCAGAATGACGGTACGACGAC |
| | | GATTGCCAATAACCTTACCAGCACGGTTCAGGTGTTTACT |
| | | GACTCGGAGTACCAGCTCCCGTACGTCCTCGGCTCGGCGC |
| | | ATCAAGGATGCCTCCCGCCGTTCCCAGCAGATGTCTTCAT |
| | | GGTGCCACAGTATGGATACCTCACCCTGAACAACGGGAGT |
| | | CAGGCAGTAGGACGCTCTTCATTTTACTGCCTGGAGTACTT |
| | | TCCTTCTCAGATGCTGCGTACCGGAAACAACTTTACCTTCA |
| | | GCTACACTTTTGAGGACGTTCCTTTCCACAGCAGCTACGCT |
| | | CACAGCCAGAGTCTGGACCGTCTCATGAATCCTCTCATCG |
| | | ACCAGTACCTGTATTTCTTAAGCAGAACAAACACTCCAAG |
| | | TGGAACCACCACGCAGTCAAGGCTTCAGTTTTCTCAGGCC |
| | | GGAGCGAGTGACATTCGGGACCAGTCTAGGAACTGGCTTC |
| | | CTGGACCCTGTTACCGCCAGCAGAGAGTCTCAAAAACAGA |
| | | CGGCGAGAACAACAACAGTGACTTCTCCTGGACAGGAGCT |
| | | ACCAAGTACCACCTCAATGGCAGAGACTCTCTGGTGAATC |
| | | CGGGACCGGCCATGGCAAGCCACAAGGACGATGAAGAAA |
| | | AGTTTTTTCCTCAGAGCGGGGTTCTCATCTTTGGGAAGCAA |
| | | AGCGCCGCCGGAGCCGATGTCGCGATCGACAGCGTCATGA |
| | | TTACAGACGAAGAGGAAATCAGGACAACCAATCCCGTGG |
| | | CTACGGAGCAGTATGGTTCTGTATCTACCAACCTCCAGAG |
| | | AGGCAACAGACAAGCAGCTACCGCAGATGTCAACACACA |
| | | AGGCGTTCTTCCAGGCATGGTCTGGCAGGACAGAGATGTG |
| | | TACCTTCAGGGGCCCATCTGGGCAAAGATTCCACACACGG |
| | | ACGGACATTTTCACCCCTCTCCCCTCATGGGTGGATTCGGA |
| | | CTTAAACACCCTCCTCCACAGATTCTCATCAAGAACACCC |
| | | CGGTACCTGCGAATCCTTCGACCACCTTCAGTGCGGCAAA |
| | | GTTTGCTTCCTTCATCACACAGTACTCCACGGGACAGGTC |
| | | AGCGTGGAGATCGAGTGGGAGCTGCAGAAGGAAAACAGC |
| | | AAACGCTGGAATCCCGAAATTCAGTACACTTCCAACTACA |
| | | ACAAGTCTGTTAATGTGGACTTTACTGTGGACACTAATGG |
| | | CGTGTATTCAGAGCCTCGCCCCATTGGTACCAGATTCCTG |
| | | ACTCGTAATCTGTAA |
| R-AGTC-0059 | 39 | MAADGYLPDWLEDTLSEGIRQWWKLKPGPPPPKPAERHKD |
| (amino acid) | | DSRGLVLPGYKYLGPFNGLDKGEPVNEADAAALEHDKAYD |
| Also referred to | | RQLDSGDNPYLKYNHADAEFQERLKEDTSFGGNLGRAVFQA |
| herein as: | | KKRVLEPLGLVEEPVKTAPGKKRPVEHSPVEPDSSSGTGKAG |
| OMY-917 | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSNAGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYFLSRTNTPS |
| | | GTTTQSRLQFSQAGASDIRDQSRNWLPGPCYRQQRVSKTDG |
| | | ENNNSDFSWTGATKYHLNGRDSLVNPGPAMASHKDDEEKF |
| | | FPQSGVLIFGKQSAAGADVAIDSVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRFLTRNL |

### Methods

Animals: P3-P5 Sprague Dawley rat pups were used for all explant experiments. Cynomolgus monkeys (non-human primates, “NHP”), age 3-5 years, were pre-screened for AAV neutralizing antibodies and then dosed bilaterally (10¹⁰ vg/ear in 30 μL volume) via injection into the cochlea via the round window membrane (RWM). NHP were euthanized and cochlear sections were evaluated 12 weeks after AAV administration for expression of GFP by immunohistochemistry.

Cochlear explants: Day 0: Dissected whole cochleae were mounted onto Cell-Tak-coated mesh inserts and incubated overnight in growth medium with antibiotics. Day 1: Cochleae were transferred to antibiotic free media supplemented with 2% FBS and treated with AAV (range of concentrations) for 120 hr continuously. Day 6: Cochleae were fixed in 4% PFA overnight and then immunostained with Phalloidin, anti-GFP, and DAPI.

Adeno-associated viruses: All capsid variants used the CBA promoter to drive expression of a Green Fluorescent Protein (GFP) reporter construct to allow for rapid and easily quantifiable assessment of tropism.

GFP quantification: Z-stacks from the middle region of the cochlea were imaged at 63× and stitched together using Zeiss Zen Black software. A region of interest box was drawn for the three relevant regions (spiral ligament, organ of Corti, and spiral limbus). GFP/anti-GFP pixel density above threshold was measured for each of the three areas. The unit of pixel density measurement is arbitrary.

### Comparison of Capsid Variant Tropism in Rat Explants

Methods for cochlear explants: P3-P5 Sprague Dawley rat pups were used for all ex vivo studies. Day 0: Dissected whole cochleae were mounted onto Cell-Tak-coated mesh inserts and incubated overnight in growth medium with antibiotics. Day 1: Cochleae were transferred to antibiotic free media supplemented with 10% FBS and treated continuously with 2e10 vg AAV for 120 hrs. Day 6: Cochleae were fixed in 4% PFA overnight then immunohistochemically processed with the following: Phalloidin (1:500), anti-GFP (1:250), and DAPI (1:1000) then mounted in anti-fade mounting media. GFP transduction was measured from three regions of interests placed over the organ of Corti, spiral limbus, or spiral ligament. Anti-GFP pixel density above threshold was measured for each region over a z-series and data presented are in arbitrary units.

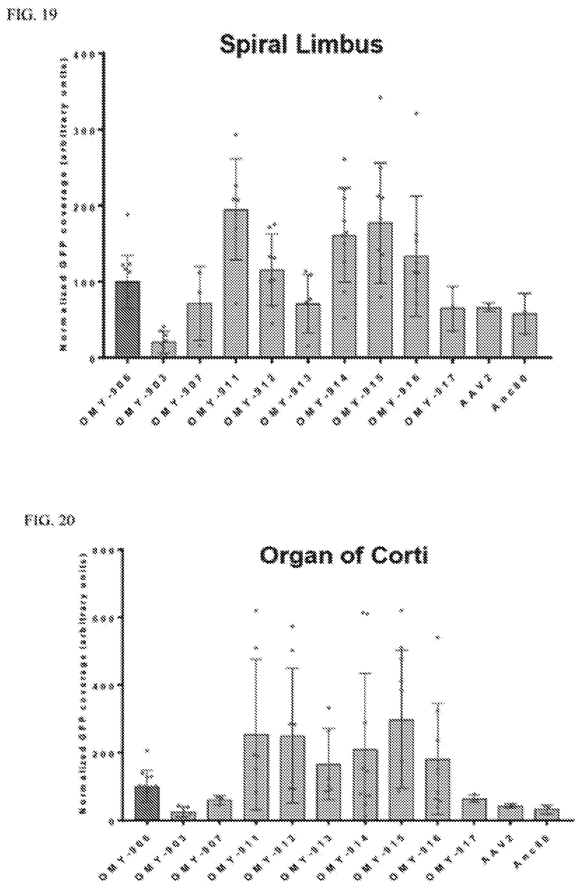

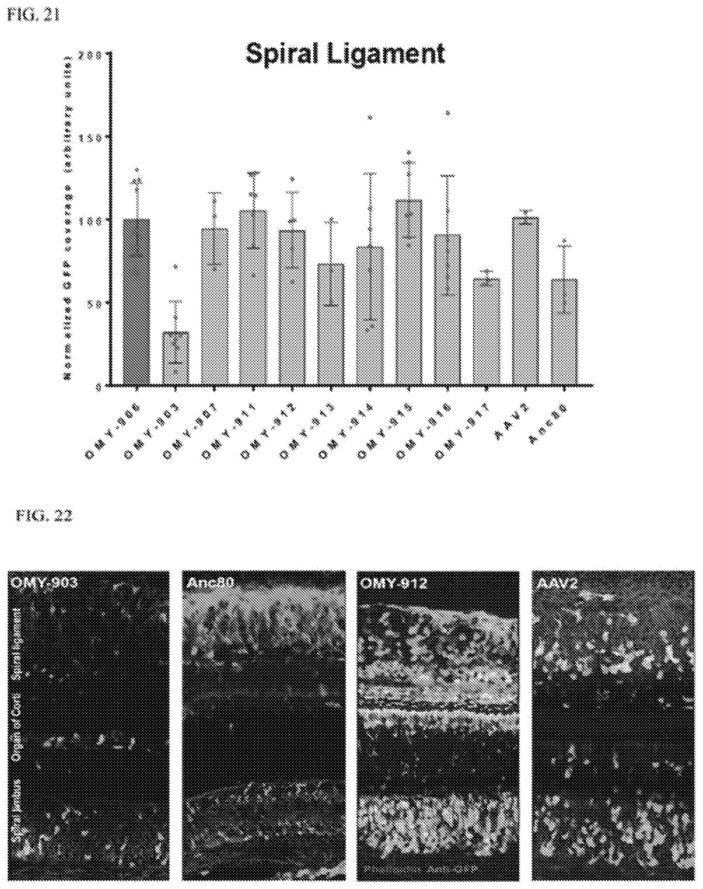

Results: FIGS. 19-21 show a comparison of AAV capsid variant GFP coverage normalized to OMY-906 (gray bars) in the spiral limbus (FIG. 19), in the Organ of Corti (FIG. 20), and in the spiral ligament (FIG. 21). All capsid variants were treated at a dose of 2e10 vg. FIG. 22 shows representative images as blended z-stacks to highlight the spiral ligament, organ of Corti support cell layer, and spiral limbus. Capsids OMY-911, OMY-912, OMY-914, and OMY-915 were among the top performers overall.

Overall, these data demonstrate that in cochlear explants, the novel AAV capsids exhibit high levels of transduction in comparison to AAV-Anc80 and wildtype AAV2.

### Comparison of Capsid Variant Tropism at Different Doses

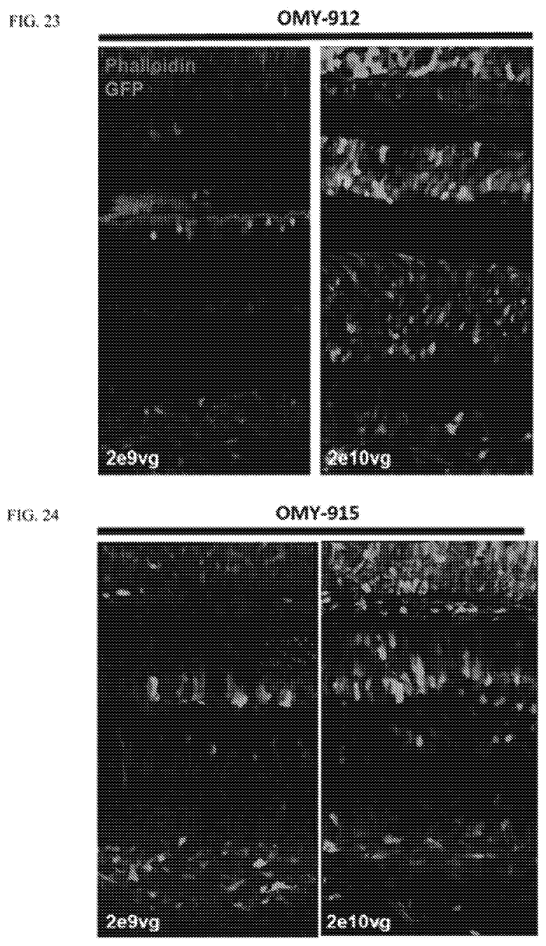

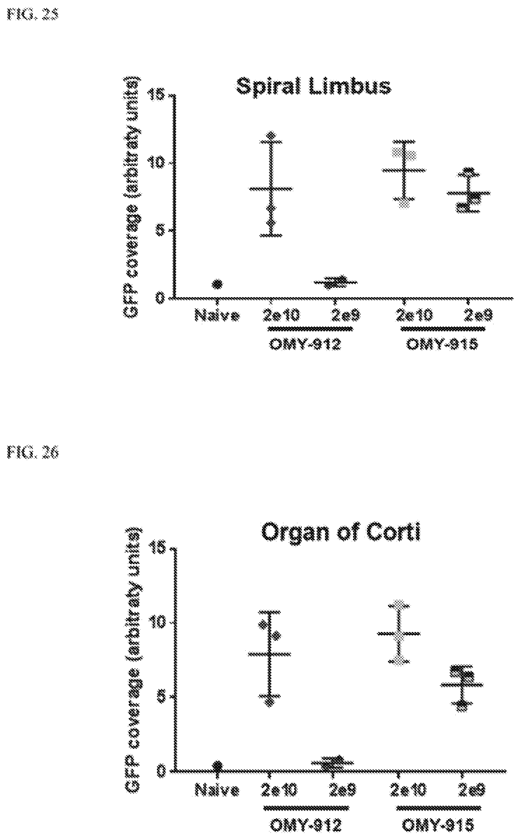

FIGS. 23-26 show a comparison of AAV capsid variant GFP coverage at different dosages. FIG. 23 shows fluorescent images comparing OMY-912 capsid variant GFP coverage at two doses: 2e9 vg and 2e10 vg. FIG. 24 shows fluorescent images comparing OMY-915 capsid variant GFP coverage at two doses: 2e9 vg and 2e10 vg. FIG. 25 shows a bar graph comparing OMY-912 and OMY-915 capsid variant GFP coverage in the spiral limbus. FIG. 26 shows a bar graph comparing OMY-912 and OMY-915 capsid variant GFP coverage in the organ of Corti. These data show that OMY-915 had higher overall GFP coverage at both doses tested.

### Expression of Connexin 26 Protein in Rat Cochlear Explants Exposed to AAV-GJB2-Flag

Methods: Cochlear explants from P2-P8 rats were treated with media containing an AAV vector carrying a FLAG-labeled version of the GJB2 gene. Explants were fixed 48-96 hours after treatment and immunostained with antibodies against Connexin 26 and FLAG; tissues were also stained with phalloidin and DAPI to label nuclei.

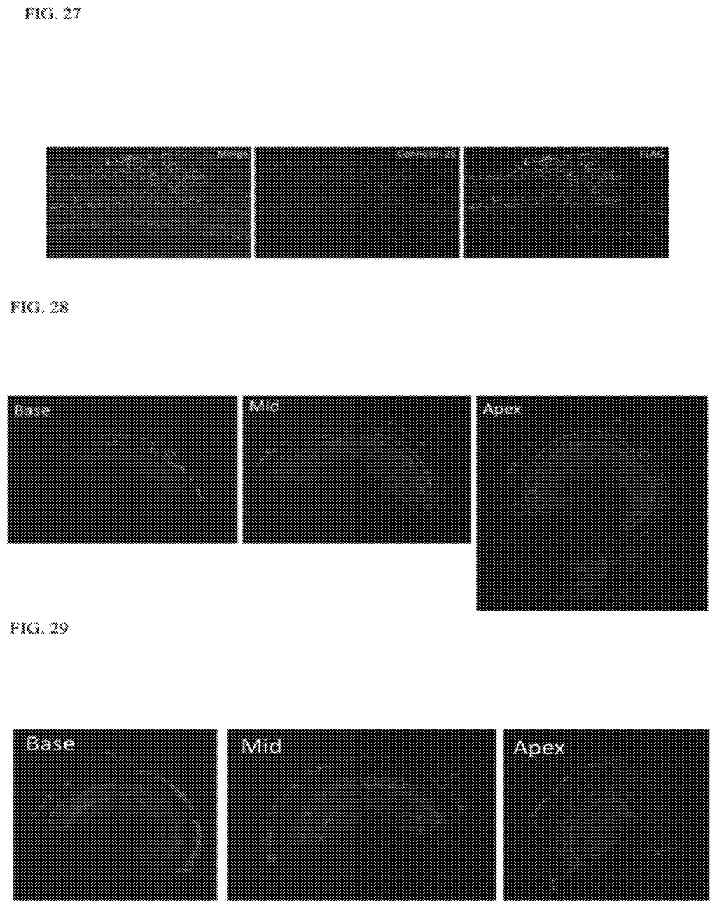

Results: FIG. 27 shows representative images of a cochlear explant exposed to OMY-914 capsid variant expressing FLAG-labeled connexin 26 showing that this virus properly delivers connexin 26 protein to the membranes and gap junction plaques of cochlear supporting cells such as in the organ of Corti, spiral limbus and spiral ligament. FLAG staining clearly overlapped with areas of connexin 26 expression demonstrating that the FLAG labeled protein is targeted to normal sites of connexin 26 expression. These data demonstrate that AAV-GJB2-Flag correctly delivers FLAG-labeled connexin 26 protein to the support cells of the cochlea ex vivo and overlaps with endogenous connexin 26 expression.

Expression of Connexin 26 Protein after Intracochlear Injection of AAV-GJB2-Flag In Vivo in Juvenile or Adult Mice

Methods: Deeply anesthetized postnatal day 8 (P8) or adult C57BL/6J mice were injected intracochlearly through the round window membrane with 1.0 μl of AAV containing a FLAG-labeled version of the GJB2 gene at a titer of 1e12 vg/mL. Mice were euthanized at age 2-6 weeks after injection and cochleae were fixed and immunostained with antibodies against Connexin 26 and FLAG; tissues were also stained with phalloidin and DAPI to label nuclei.

Results: FIG. 28 shows a representative example of intracochlear injection in young mice (P8) OMY-914 capsid variant containing FLAG-labeled connexin 26 showing that this gene therapy product properly delivers connexin 26 protein to the membranes and gap junction plaques of cochlear supporting cells such as in the organ of Corti, spiral limbus and spiral ligament.

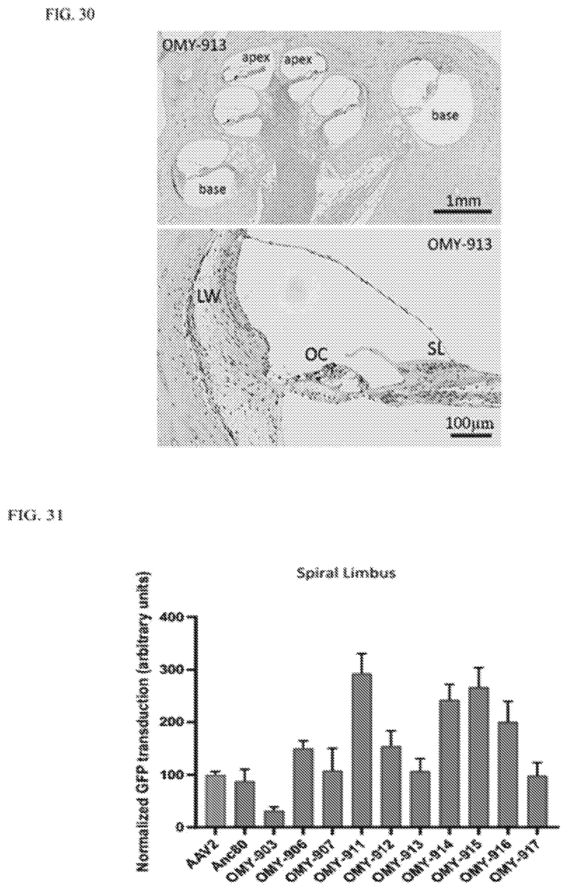

FIG. 29 shows a representative example of intracochlear injection in adult mice (2-3 months of age) of OMY-914 capsid variant expressing FLAG-labeled connexin 26 showing that this viral construct properly delivers connexin 26 protein to the membranes and gap junction plaques of adult cochlear supporting cells such as in the organ of Corti, spiral limbus and spiral ligament.

These data demonstrate that AAV-GJB2-Flag correctly delivers Connexin 26 protein to the support cells of the cochlea in vivo.

### AAV Delivery and Tropism In Vivo in Non-Human Primates

Methods for non-human primate (NHP) tropism study: Cynomolgus monkeys (“NHPs”), age 3-5 years, were pre-screened for AAV neutralizing antibodies and then dosed bilaterally (10¹⁰ vg/ear in 30 μL volume) via injection into the cochlea via the round window membrane (RWM). NHPs were euthanized and cochlear sections were evaluated 12 weeks after AAV administration for expression of GFP by immunohistochemistry.

Results: Non-human primate (NHP) cochleae were evaluated by immunohistochemistry 12 weeks after intracochlear injection of AAVs (FIG. 30). In FIG. 30 DAB staining for GFP expression has been pseudocolored red. FIG. 30 (Top panel) shows low magnification image of the entire cochlea and demonstrates that consistent expression from OMY-913 that can be observed from base to apex throughout the cochlea after a single AAV intracochlear injection administered near the base via round window membrane (RWM) injection. FIG. 30 (Bottom panel) shows that OMY-913 expression is observed in the regions relevant to GJB2 rescue, including the lateral wall (LW), organ of Corti (OC) support cells, and spiral limbus (SL).

Overall, these data demonstrate that AAVs described herein, in some embodiments, are capable of transducing GJB2-relevant cells throughout the NHP cochlea after a single intracochlear injection through round window membrane (RWM).

Based on those results, and without wishing to be bound by any particular theory, it was contemplated herein that AAV capsid variants with similar transduction efficiencies at high doses may show different transduction efficiencies at lower doses. Further, the AAV capsid variants exhibit high levels of GFP coverage in cochlear explants in comparison to AAV-Anc80. Anc80 exhibits a different tropism pattern in rat compared to mouse explants. Additionally, the AAV capsid variants were capable of transducing GJB2-relevant cells throughout the NHP cochlea after a single RWM injection, including support cells of the organ of Corti and spiral limbus, and fibrocytes of the spiral ligament.

### Example 2: AAV-Mediated GJB2 Gene Therapy Rescues Hearing Loss and Cochlear Damage in Mouse Models of Congenital Hearing Loss Caused by Conditional Connexin26 Knockout

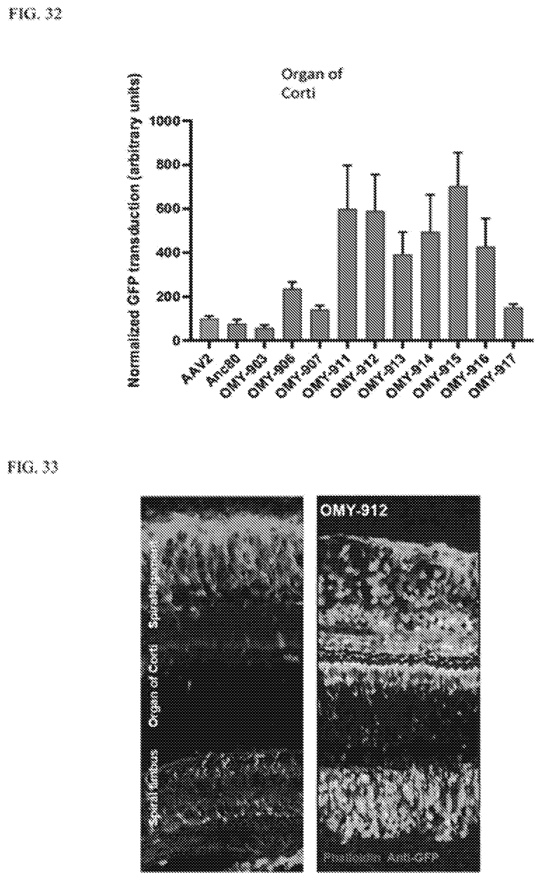

Results from various mouse and human studies have revealed that mutations in Cx26 can ultimately lead to near total degeneration of cochlear hair cells. Since the constitutive homozygous Cx26 knockout is embryonic lethal, we utilized conditional knockouts to study the effect of losing CX26 protein in the cells of the inner ear. We utilized two different conditional knockout strains (Cx26 cKO) generated by crossing Cx26^{loxp/loxp} mice with either an inducible cre mouse line or with a constitutive cre mouse line. Using the inducible cre line, we knocked out Cx26 with temporal control and observed varying degrees of hearing loss and developmental defects dependent on the time of cre induction. Early postnatal cre induction caused severe to profound hearing loss in the Cx26 cKO mice when assessed at postnatal day 30 (FIG. 31), whereas later induction of cre resulted in mild to moderate hearing loss that was progressive in nature. Constitutive cre Cx26 cKO animals, by virtue of embryonic cre expression in the inner ear tissues, displayed severe to profound hearing loss across the 4, 8, 16, 32 and 48 kHz frequencies (FIG. 32). The availability of these various mouse models enabled us to evaluate AAV-mediated GJB2 gene therapy across a spectrum of hearing loss severity that mimics known human phenotypes. An example AAV-GJB2 gene therapeutic (“THERAPEUTIC A”) was constructed with one of the top AAV capsid performers in Example 3, a promotor selected from Sequence IDs 1-6, and GJB2co369 as the gene to be delivered.

In experiments designed to evaluate the ability of THERAPEUTIC A to rescue the Cx26 cKO phenotype, we performed intracochlear injection via the posterior semicircular canal (PSCC) route of THERAPEUTIC A or vehicle into both models of Cx26 cKO mice postnatally. Compared with vehicle, administration of THERAPEUTIC A to inducible cre Cx26 cKO animals substantially restored CX26 expression and provided a marked improvement in hearing across multiple frequencies as measured by ABR. In addition, THERAPEUTIC A-injected Cx26 cKO mice had greatly improved cochlear morphology relative to those injected with vehicle, corresponding with the ABR data (FIG. 33). Sub-cellular localization of the CX26 protein in rescued animals was normal and apparent in inner sulcus, Claudius, Hensen, pillar, and Deiters cells as well as in the spiral prominence, and fibrocytes of the spiral limbus and lateral wall, and these animals showed increased numbers of surviving hair cells relative to vehicle treated controls.

### Example 3. Further Characterization of Novel AAV Capsid Variants for Delivery of GJB2 Gene Therapy for Congenital Hearing Loss

Mutations in over 100 genes have been causally linked to hearing loss. Adeno-associated viruses (AAVs) have been shown to be safe and effective delivery vectors for gene therapy with a track record of positive clinical outcomes. AAV capsids represent critical regulatory elements that influence tropism. This study evaluates novel and previously described AAV capsid variants for ideal tropism in cochlear explants and non-human primates (NHP) studies to identify optimal capsids for GJB2 gene therapy. We designed an AAV vector with an optimized capsid, promoter and human GJB2 gene elements (THERAPEUTIC A) that provides excellent expression of CX26 in cochlear support cells and fibrocytes. We also generated an identical AAV vector that expresses CX26 with a FLAG-tag to allow identification of virally expressed CX26 (THERAPEUTIC A-FLAG). The top performing capsid was then packaged with the GJB2 transgene (THERAPEUTIC A) and pharmacodynamics of the connexin 26 (CX26) protein was evaluated following in vivo administration. As described herein, in cell-based assays, utilizing HeLa cells that do not normally express CX26, both THERAPEUTIC A and THERAPEUTIC A-FLAG induced expression of CX26 that was correctly trafficked to the cell membrane. Additionally, injection of THERAPEUTIC A-FLAG into the cochleae of mice provided near total expression of CX26-FLAG in the cells of interest throughout the cochlea (from base to apex).

### Therapeutic a Enhances FRAP Signal in HeLa Cells

Methods for FRAP assay: HeLa cells were seeded into 96-well plates at a density of 20,000 cells/well. After 24 hours, the cells were transduced with AAV vectors (MOI=10,000) and Adenovirus serotype 5 (MOI=5). The FRAP assay was conducted after an additional 48 hours to allow for transgene expression. For the FRAP assay, cells were incubated with Calcein-AM (2.5 uM) for 30 minutes, washed in HBSS, and then imaged on the Operetta high-content imaging system. The center field was photobleached using the 40× objective and exposure to 488 nm fluorescent illumination (5000 ms×12 repetitions). Immediately afterwards, the wells were imaged at 10× magnification for 30 minutes. Fluorescence recovery was measured as the difference in 488 nm intensity between the photobleached region and the surrounding, unbleached region, which was then normalized to the surrounding unbleached region to account for fluctuations in light intensity from the Xenon arc lamp fluorescent light source.

A buffered solution of THERAPEUTIC A for intracochlear administration is used in the following experiments. The THERAPEUTIC A construct can optionally include a FLAG tag.

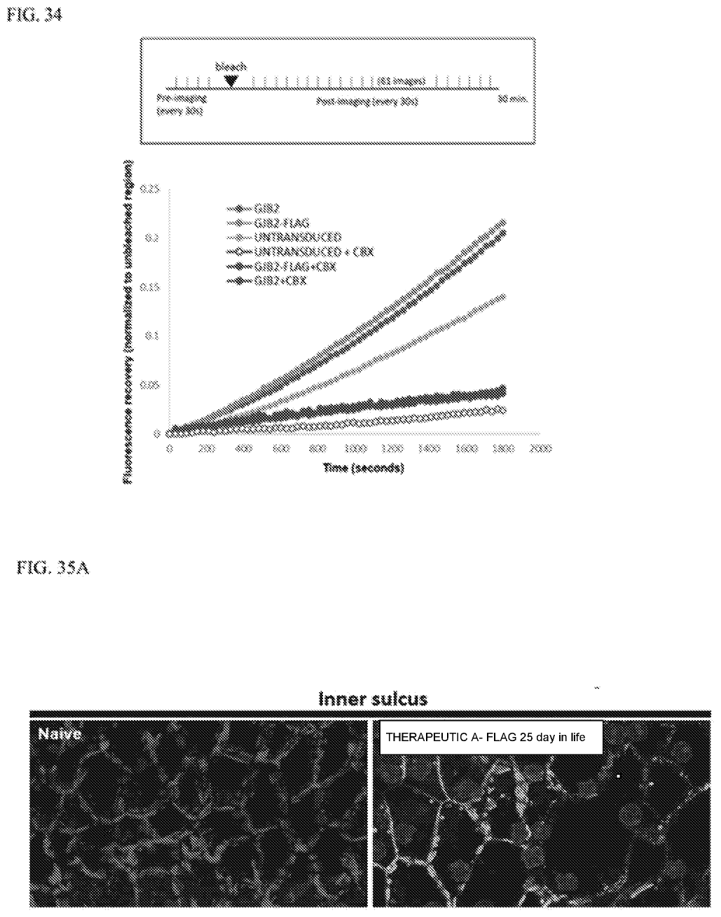

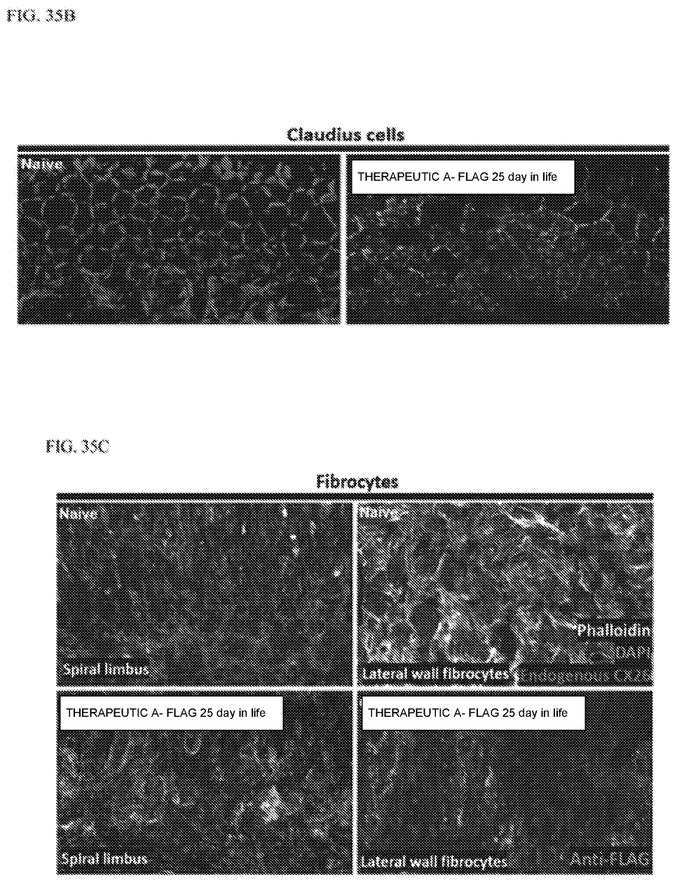

Results: HeLa cells, which do not natively express CX26, were used to evaluate the functionality of THERAPEUTIC A driven CX26 expression. Prior to photobleaching, HeLa cells were co-incubated with THERAPEUTIC A and Ad5 for 5 hours followed by a 48-hour recovery to allow for transgene expression. Calcein-AM hydrolyzes upon cellular uptake to become fluorescent and membrane impermeable. Intracellular Calcein dye is known to transfer to adjacent cells via functional gap junctions. The pan gap junction inhibitor carbenoxolone (CBX) was used to determine the contribution of non-gap junction mediated fluorescence recovery. FIG. 34 (top panel) shows a timeline of photobleaching and image capture for each FRAP trial. Fluorescence recovery was measured for 30 minutes post photobleaching. FIG. 34 (bottom panel) shows both THERAPEUTIC A and THERAPEUTIC A-FLAG recover fluorescence faster than untransduced HeLa cells signifying that the transgene driven protein is likely forming functional gap junctions. The addition of carbenoxolone reduces most of the fluorescence recovery indicating that functioning gap junctions are the major contributor of cell to cell dye transfer.

Overall, these data demonstrate that THERAPEUTIC A mediated delivery of GJB2 or GJB2-FLAG into HeLa cells, which do not natively express CX26, enhances Calcein-AM dye FRAP signal indicating that the GJB2 transgene forms functional gap junctions.

### Tropism of THERAPEUTIC a Following PSCC Delivery in P6 Mouse Pups

Methods for pup injections: P6 C57BL/6J mouse pups were anesthetized via mild hypothermia and injected with 1 μL of THERAPEUTIC A-FLAG via the posterior semicircular canal (PSCC). Mice were sacrificed and perfused at 25 days post injection and cochleae were harvested for downstream immunohistochemical processing. Detection of virally-expressed CX26-FLAG was made using a FLAG antibody. Cochleae were processed with the following antibodies or stains: Phalloidin (1:500), anti-FLAG (1:250), anti-CX26 (1:250), and DAPI (1:1000).

Results: FIGS. 35A-35C show intracochlear injection of THERAPEUTIC A-FLAG (green) via the posterior semicircular canal (PSCC) in P6 mouse pups exhibits a high degree of transduction relative to endogenous CX26 expression (magenta). CX26-FLAG expression is present at high levels throughout the length of the cochlea and forms membranous, plaque-like structures in the inner sulcus (FIG. 35A), Claudius cells (FIG. 35B), and other support cell types (FIG. 35C). CX26-FLAG expression is also present in fibrocytes of the spiral limbus and lateral wall, and is consistent with the morphology and pattern of endogenous CX26 expression.

Overall, these data demonstrate that direct intracochlear delivery of THERAPEUTIC A-FLAG into P6 mouse pups via PSCC injection results in broad CX26-FLAG transduction in cell types that natively express CX26.

### Safety and Tropism Profile of THERAPEUTIC a Following RWM+PSCC Fenestration Intracochlear Delivery in P30 Adult Mice

Methods for adult injections: Adult (P30) C57BL/6J mice were injected with 1 μL of either THERAPEUTIC A or THERAPEUTIC A-FLAG via direct intracochlear injection through the round window membrane (RWM). Prior to injection, a fenestration was made in the posterior semicircular canal (PSCC) to allow fluid flow. Mice were sacrificed and cardiac perfused with 4% PFA to fix the tissues at either 14 or 42 days post-injection, and cochleae were harvested for downstream immunohistochemical processing. Cochleae were processed with the following antibodies or stains: Phalloidin (1:500), anti-FLAG (1:250), anti-CX26 (1:250), and DAPI (1:1000).

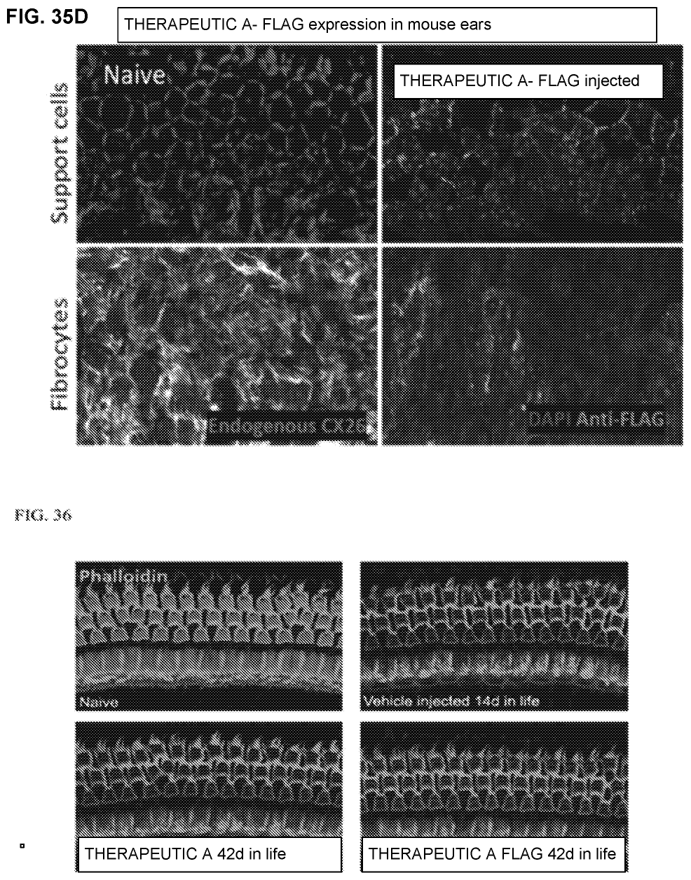

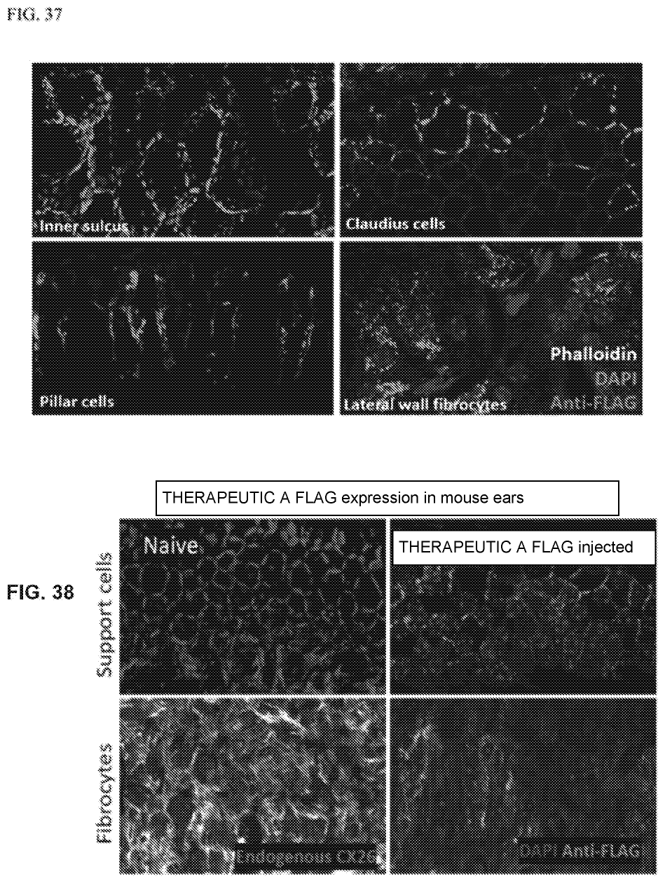

Results: FIG. 36 shows that intracochlear injection of THERAPEUTIC A or THERAPEUTIC A-FLAG via the round window membrane with fenestration in the posterior semicircular canal of adult mice at age P30 was safe and did not cause damage to the inner or outer hair cells at 42 days post-surgery. FIG. 37 and FIG. 38 show CX26-FLAG transduction (green) in the inner sulcus, Claudius cells and lateral wall fibrocytes cells at 14 days post-surgery. CX26-FLAG expression in the inner sulcus and Claudius cells is membranous and forms plaque-like structures similar to endogenous CX26.

Overall these data demonstrate that adult intracochlear delivery of THERAPEUTIC A-FLAG via direct intracochlear injection through the round window membrane (RWM) with posterior semicircular canal (PSCC) fenestration results in transduction of CX26-FLAG with a clean safety profile.

### Example 4. Rescue of Hearing Loss and Cochlear Degeneration in a Clinically Relevant Inducible Mouse Model of GJB2 Congenital Hearing Loss

GJB2 gene mutations cause the most common form of congenital non-syndromic deafness in humans. GJB2 encodes the gap junction protein Connexin 26 (CX26), required in the inner ear for the function of non-sensory cells such as support cells and fibrocytes. In general, the onset of hearing loss is prelingual and moderate to severe, however, in some subjects, hearing loss due to loss of CX26 can be mild and progressive. Human temporal bone studies have revealed degeneration of hair and support cells in GJB2 mutant cochleae, whereas spiral ganglion neurons remain primarily unaffected. In this study, THERAPEUTIC A, an AAV-based gene therapy candidate, is evaluated in an inducible mouse model of GJB2-deficiency.

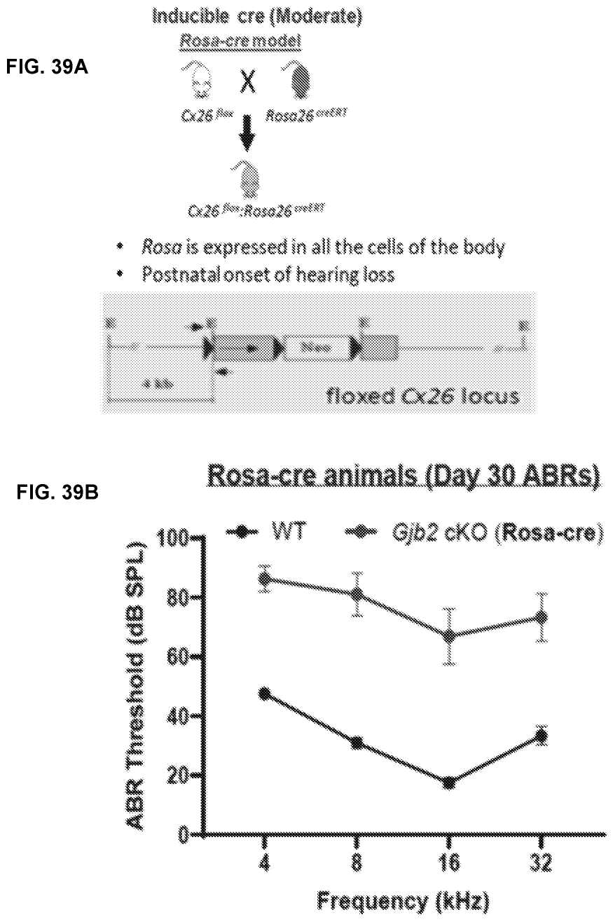

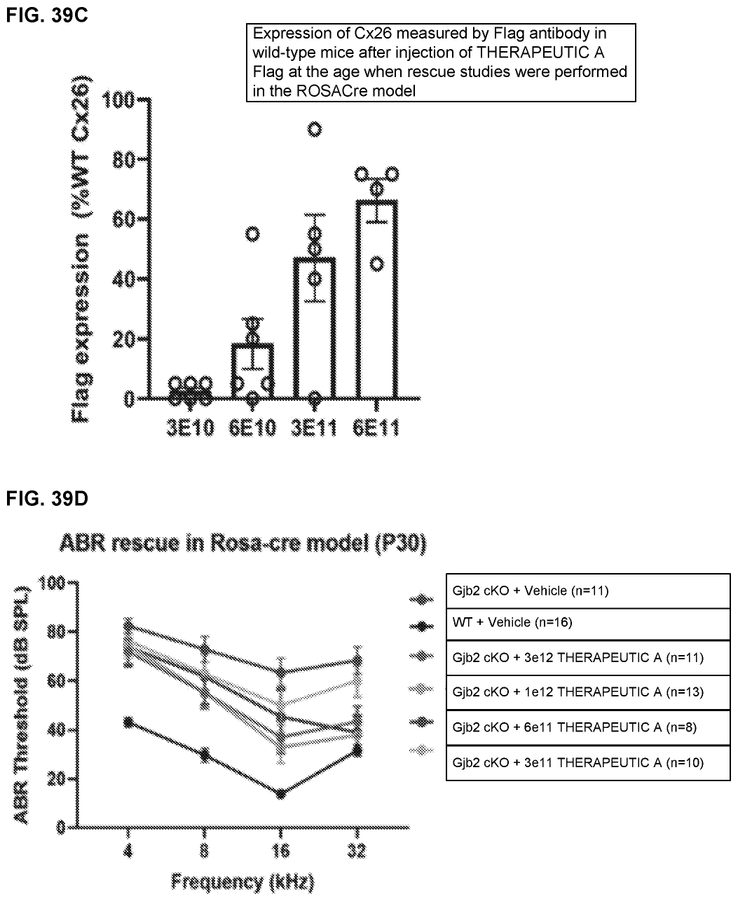

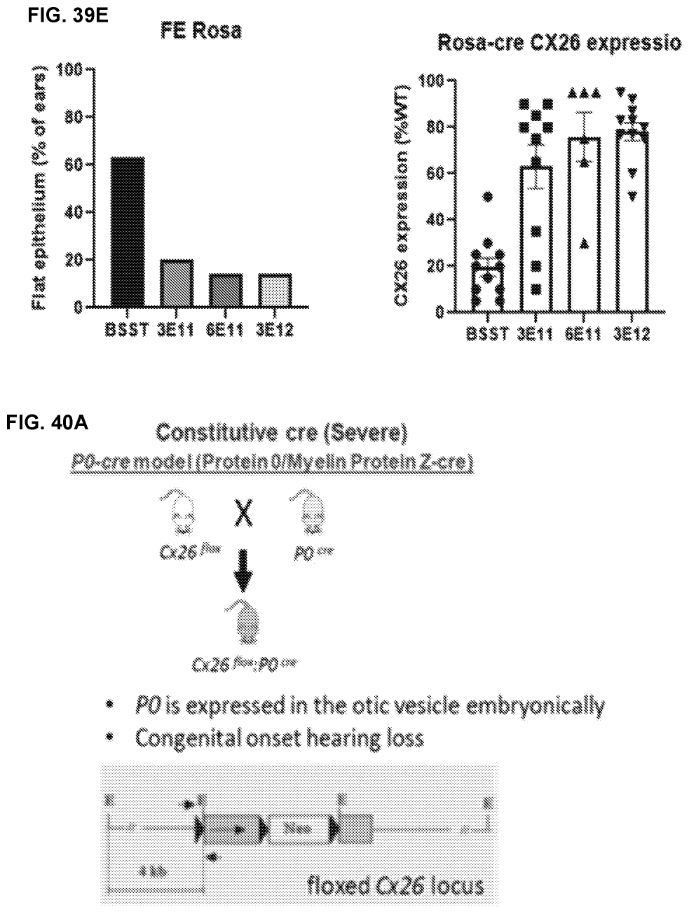

Methods: Since homozygous Cx26 knockout is embryonic lethal in mice, we utilized Cx26 conditional knockout (Cx26 cKO), generated by crossing Cx26^{loxp/loxp} mice with a tamoxifen inducible cre (Rosa-cre^{ER}) mouse line to study the effect of losing Cx26 protein in the cells of the inner ear, as illustrated in FIGS. 39A and 39B. In the Cx26flox animal, the coding region in exon 2 was flanked by a loxP site in intron 1 and a floxed neo cassette inserted into exon 2. Further, we developed an AAV based gene therapy candidate (THERAPEUTIC A) after screening different capsids, promoters, and optimized GJB2 codons. We also created THERAPEUTIC A-FLAG that expresses a FLAG-tagged CX26 and administered it via the intracochlear (IC) route in wildtype animals to determine the tropism of AAV derived CX26 in the inner ear by tracking FLAG expression. To study the efficacy of gene therapy, THERAPEUTIC A or vehicle were administered to Cx26 cKO mice postnatally via the IC route. Auditory Brainstem Responses were measured at postnatal day (P) 30, and the cochleae were processed for histology to determine the morphology and CX26 expression.

Results: Adjusting the timing of tamoxifen administration allowed temporal control of Cx26 knockout, resulting in varying degrees of hearing loss and cochlear defects dependent on the time of cre activation. Early postnatal cre activation caused severe to profound hearing loss in the Cx26 cKO mice at P30, whereas later cre activation caused a progressive mild to moderate type of hearing loss. Histological examination revealed little to no Cx26 expression in the Cx26 cKO mice. As shown in FIG. 39C, intracochlear injection of THERAPEUTIC A-FLAG into wildtype mice during the postnatal period provides extensive cochlear coverage including all cell types that natively express CX26. Intracochlear (IC) administration of THERAPEUTIC A-FLAG to naïve mice confirmed AAV transduction in the support cells and fibrocytes. As shown in FIGS. 39D and 39E, Cx26 cKO animals injected with THERAPEUTIC A demonstrated substantial rescue of ABR thresholds across multiple frequencies, restoration of CX26 expression and preservation of cochlear morphology, relative to vehicle injected Cx26 cKOs.

Based on those results, and without wishing to be bound by any particular theory, it is contemplated in this disclosure that intracochlear administration of THERAPEUTIC A successfully restored hearing, the expression of CX26 in the relevant cochlear cell types and rescued cochlear morphology in a Cx26 cKO mouse models that mimic, in part, the auditory deficits found in human GJB2 patients.

### Example 5. Rescues Hearing Loss and Cochlear Degeneration in a Clinically Relevant Mouse Model of GJB2 Congenital Hearing Loss

GJB2 mutations represent the most common cause of genetic hearing loss in humans. GJB2 encodes for connexin 26 (CX26) a gap junction protein that is natively expressed in fibrocytes of the spiral limbus and spiral ligament as well as supporting cells within the organ of Corti. Results from mouse and human studies have shown that GJB2 mutations lead to elevated auditory brain response (ABR) thresholds and degeneration of supporting and hair cells. To rescue this GJB2 deficient phenotype we sought to deliver functional copies of GJB2 via intracochlear administration of AAV. We first identified a novel class of AAV capsids that efficiently transduce cochlear cell types that natively express CX26. We then further optimized the AAV construct by packaging the novel capsid, promoter, and human GJB2 gene elements with and without a Flag tag (THERAPEUTIC A-Flag and THERAPEUTIC A, respectively). Here, we have utilized a constitutive Cre mouse model of GJB2 hearing loss to evaluate the therapeutic potential of THERAPEUTIC A.

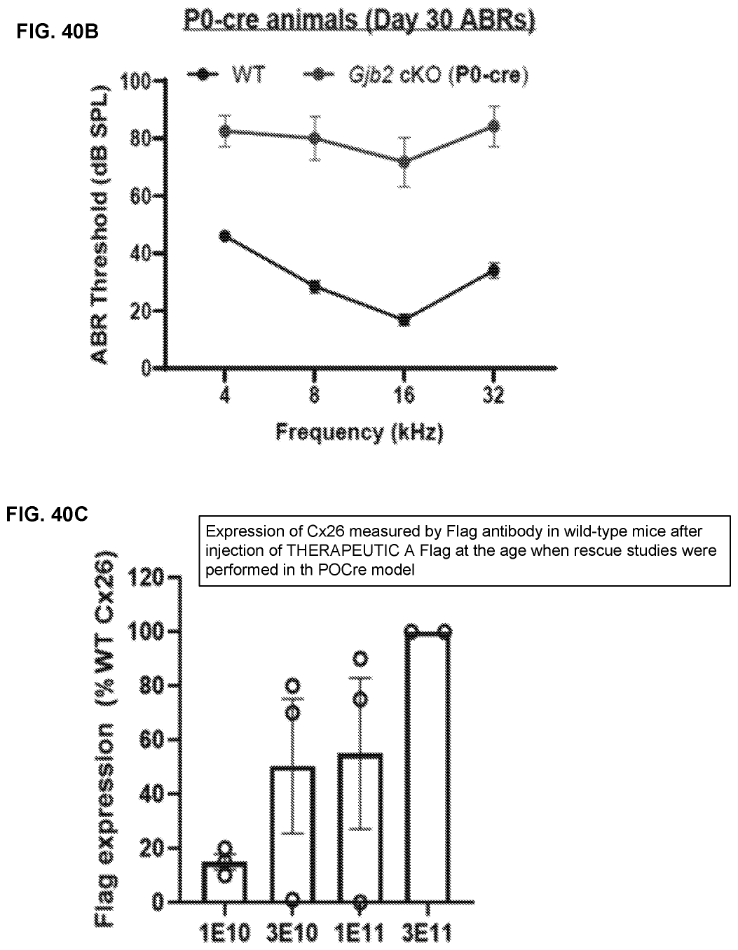

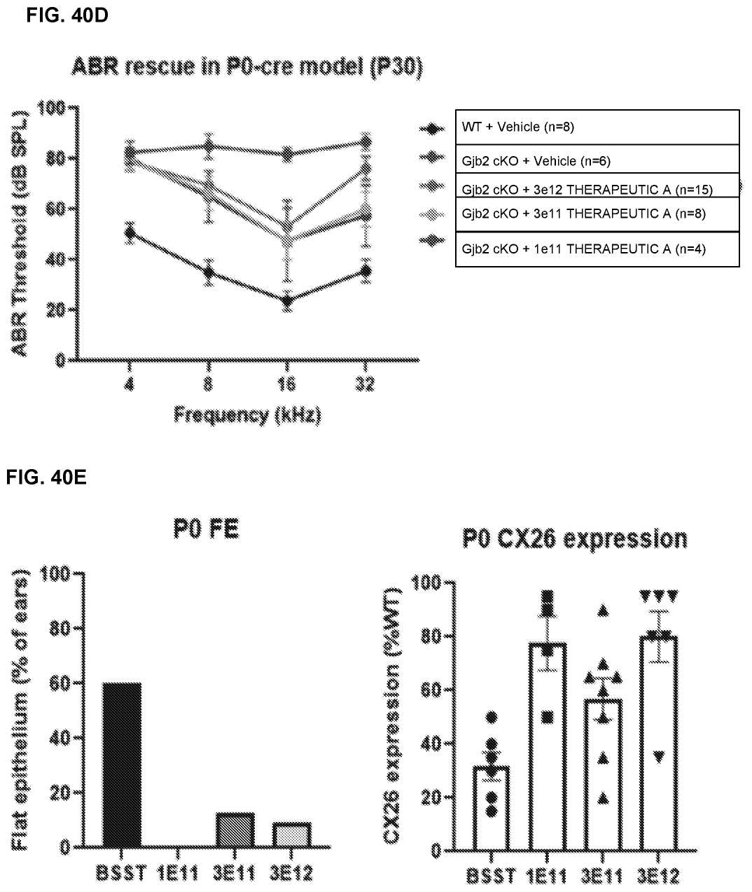

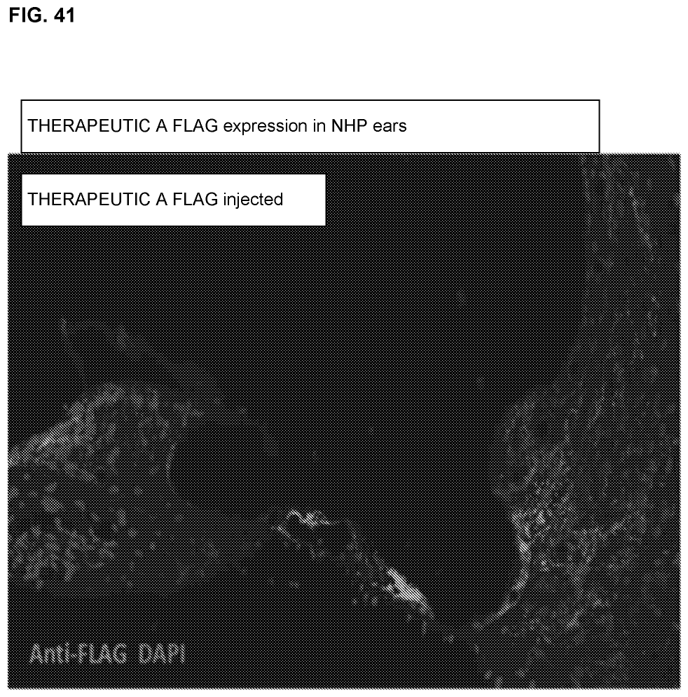

Methods: To assess in vivo rescue of CX26 deficiency we generated a mouse model with inner ear deletion of GJB2 by crossing Cx26^{loxp/loxp} mice with mice expressing Cre driven by the inner ear specific promoter P0 (P0-Cre), as illustrated in FIGS. 40A and 40B. In the Cx26flox animal, the coding region in exon 2 was flanked by a loxP site in intron 1 and a floxed neo cassette inserted into exon 2. The onset of P0-Cre occurs embryonically and previous studies have reported disrupted plaque formation as early as E14.5 in this model. Postnatal mice were injected with 1 μL THERAPEUTIC A, THERAPEUTIC A-FLAG, or vehicle via the posterior semicircular canal and later assessed for various efficacy endpoints as early as P30. Auditory sensitivity was measured by ABR after which cochleae were collected and immunohistochemically processed with anti-CX26, anti-FLAG, and phalloidin to assess for tropism and cochlear morphology. For evaluation of tropism, cochlear and lateral wall whole mounts were imaged on a Zeiss LSM880 confocal microscope and FLAG or CX26 coverage was quantified.

Results: As shown in FIG. 40C, intracochlear injection of THERAPEUTIC A-FLAG into wildtype mice during the postnatal period provides extensive cochlear coverage including all cell types that natively express CX26. P0-Cre mice exhibit a substantial reduction in CX26 expression and the presence of a flat epithelium phenotype where there is a complete loss of hair cells and supporting cells and severe to profound hearing loss. As shown in FIGS. 40D and 40E, intracochlear administration of THERAPEUTIC A to P0-Cre mice substantially restored CX26 expression and greatly reduced the occurrence of a flat epithelium phenotype, increased the number of hair cells present, and more importantly demonstrated functional improvement in hearing across multiple frequencies as measured using ABRs.

Based on those results, and without wishing to be bound by any particular theory, it is contemplated in this disclosure that intracochlear injection of THERAPEUTIC A is capable of rescuing CX26 deficient hearing loss and cochlear pathologies.

### Example 6. Delivery and Tropism of THERAPEUTIC a in Non-Human Primates

Methods for non-human primate (NHP) tropism study: Cynomolgus monkeys (“NHPs”), age 3-5 years, were pre-screened for AAV neutralizing antibodies and then dosed bilaterally (10¹⁰ vg/ear in 30 μL volume) via injection into the cochlea via the round window membrane (RWM). NHPs were euthanized and cochlear sections were evaluated 12 weeks after administration of THERAPEUTIC A-FLAG for expression of CX26-FLAG by immunohistochemistry. Cochleae were processed with the following antibodies or stains: Phalloidin (1:500), anti-FLAG (1:250), anti-CX26 (1:250), and DAPI (1:1000).

Results: FIG. 41 shows that intracochlear injection of THERAPEUTIC A-FLAG exhibits a high degree of transduction. CX26-FLAG expression is present at high levels in the regions relevant to GJB2 rescue, including the lateral wall (LW), organ of Corti (OC) support cells, and spiral limbus (SL). CX26-FLAG expression is consistent with the morphology and pattern of endogenous CX26 expression.

Based on those results, and without wishing to be bound by any particular theory, in is contemplated herein that THERAPEUTIC A-FLAG is capable of transducing GJB2-relevant cells throughout the NHP cochlea after intracochlear administration.

Although the present disclosure has been described in some detail by way of illustration and example for purposes of clarity of understanding, it should be understood that certain changes and modifications may be practiced within the scope of the appended claims. Modifications of the above-described modes for carrying out the present disclosure that would be understood in view of the foregoing disclosure or made apparent with routine practice or implementation of the present disclosure to persons of skill in gene therapy, molecular biology, otology and/or related fields are intended to be within the scope of the following claims.

All publications (e.g., Non-Patent Literature), patents, patent application publications, and patent applications mentioned in this specification are indicative of the level of skill of those skilled in the art to which this disclosure pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

While the foregoing disclosure has been described in connection with certain preferred embodiments, it is not to be limited thereby.

1. A composition for treating or preventing hearing loss associated with deficiency of a gap junction beta-2 (GJB2) gene, wherein the composition comprises a recombinant adeno-associated virus (rAAV) virion comprising:
(i) a variant AAV capsid polypeptide which exhibits increased tropism in inner ear tissues or cells, optionally, as compared to a non-variant AAV capsid polypeptide; and
(ii) a polynucleotide comprising a nucleic acid sequence encoding the gene.

2. The composition of claim 1, wherein the variant AAV capsid polypeptide is selected from the group consisting of a variant AAV1 capsid polypeptide; a variant AAV2 capsid polypeptide; a variant AAV3 capsid polypeptide; a variant AAV4 capsid polypeptide; a variant AAV5 capsid polypeptide; a variant AAV6 capsid polypeptide; a variant AAV7 capsid polypeptide; a variant AAV8 capsid polypeptide; a variant AAV9 capsid polypeptide; a variant rh-AAV10 capsid polypeptide; a variant AAV10 capsid polypeptide; a variant AAV11 capsid polypeptide; and a variant AAV12 capsid polypeptide.

3. The composition of claim 1, wherein the variant AAV capsid polypeptide is a variant AAV2 capsid polypeptide.

4. The composition of claim 1, wherein the variant AAV capsid polypeptide comprises an amino acid sequence selected from SEQ ID NOs: 18-39, or an amino acid sequence having at least about 85%, 90%, 95%, or 99% sequence identity thereto, optionally, wherein the AAV capsid is selected from the group consisting of a VP1, VP2, and VP3 capsid polypeptide.

5. The composition of claim 4, wherein the variant AAV capsid polypeptide comprises an amino acid sequence having one or more amino acid substitutions, insertions, and/or deletions relative to a wildtype AAV2 capsid polypeptide (SEQ ID NO: 1), optionally, wherein the one or more amino acid substitutions, insertions, and/or deletions occurs at an amino acid residue selected from the group consisting of Q263, S264, Y272, Y444, R487, P451, T454, T455, R459, K490, T491, S492, A493, D494, E499, Y500, T503, K527, E530, E531, Q545, G546, S547, E548, K549, T550, N551, V552, D553, E555, K556, R585, R588, and Y730.

6. The composition of claim 4, wherein the variant AAV capsid polypeptide comprises an amino acid sequence having one or more amino acid substitutions relative to a wildtype AAV2 capsid polypeptide (SEQ ID NO: 1) selected from the group consisting of Q263N, Q263A, S264A, Y272F, Y444F, R487G, P451A, T454N, T455V, R459T, K490T, T491Q, S492D, A493G, D494E, E499D, Y500F, T503P, K527R, E530D, E531D, Q545E, G546D, G546S, S547A, E548T, E548A, K549E, K549G, T550N, T550A, N551D, V552I, D553A, E555D, K556R, K556S, R585S, R588T, and Y730F.

7. The composition of claim 1, wherein the variant AAV capsid polypeptide comprises:
(i) an amino acid sequence of any one of SEQ ID NOs: 27, 29, 31, 33, or 35;
(ii) an amino acid sequence having at least about 85%, 90%, 95%, or 99% sequence identity to any one of SEQ ID NOs: 27, 29, 31, 33, or 35; or
(iii) an amino acid sequence encoded by the nucleic acid sequence of any one of SEQ ID NOs: 26, 28, 30, 32, or 34.

8-14. (canceled)

15. The composition of claim 1, wherein the nucleic acid sequence encoding GJB2 encodes mammalian GJB2.

16. (canceled)

17. The composition of claim 1, wherein the nucleic acid sequence encoding GJB2 comprises SEQ ID NO: 10.

18. The composition of claim 1, wherein the nucleic acid sequence encoding GJB2 is codon optimized for mammalian expression.

19. The composition of claim 18, wherein the nucleic acid sequence encoding GJB2 is selected from the group consisting of: SEQ ID NO: 11, SEQ ID NO: 12 and SEQ ID NO: 13.

20. (canceled)

21. The composition of claim 1, wherein the nucleic acid sequence encoding GJB2 is a cDNA sequence.

22. The composition of claim 1, wherein the nucleic acid sequence encoding GJB2 further comprises
(i) an operably linked C-terminal tag or N-terminal tag,
(ii) a promoter,
(iii) an operably linked 3′UTR regulatory region, and/or
(iv) an operably linked polyadenylation signal.

23. The composition of claim 22, wherein the tag is a FLAG-tag or a HA-tag.

24. (canceled)

25. The composition of claim 22, wherein the promoter is selected from the group consisting of: an ubiquitously-active CBA, a small CBA promoter (smCBA), an EF1a promoter, a CASI promoter, a cochlear-support cell promoter, a GJB2 expression-specific GFAP promoter, a small GJB2 promoter, a medium GJB2 promoter, a large GJB2 promoter, a sequential combination of 2-3 individual GJB2 expression-specific promoters, and a synthetic promoter.

26. The composition of claim 22, wherein the promoter is optimized to drive sufficient GJB2 expression to treat or prevent hearing loss.

27. (canceled)

28. The composition of claim 22, wherein the nucleic acid sequence encoding GJB2 further comprises an operably linked 3′UTR regulatory region comprising a Woodchuck Hepatitis Virus Postranscriptional Regulatory Element (WPRE).

29. (canceled)

30. The composition of claim 22, wherein the polyadenylation signal is an SV40 polyadenylation signal or a human growth hormone (hGH) polyadenylation signal.

31. (canceled)

32. The composition of claim 1, wherein the polynucleotide further comprises an AAV genomic cassette, optionally, wherein:
(i) the AAV genomic cassette is flanked by two sequence-modulated inverted terminal repeats, preferably about 143-bases in length; or
(ii) the AAV genomic cassette is flanked by a self-complimentary AAV (scAAV) genomic cassette consisting of two inverted identical repeats, preferably no longer than 2.4 kb, separated by an about 113-bases scAAV-enabling ITR (ITRΔtrs) and flanked on either end by about 143-bases sequence-modulated ITRs.

33. The composition of claim 1, wherein the polynucleotide comprises a codon/sequence-optimized human GJB2 cDNA with or without a hemagglutinin C-terminal tag or a Flag tag, preferably about 27-nucleotide in length, optionally about a 0.68 kilobase (kb) in size; operably linked to one of the following promoter elements optimized to drive high GJB2 expression: (a) an ubiquitously-active CBA, preferably about 1.7 kb in size, a small CBA (smCBA), preferably about 0.96 kb in size, an EF1a, preferably about 0.81 kb in size, or a CASI promoter, preferably about 1.06 kb in size; (b) a cochlear-support cell or a GJB2 expression-specific GFAP promoter, preferably about 1.68 kb in size, a small GJB2 promoter, preferably about 0.13 kb in size, a medium GJB2 promoter, preferably about 0.54 kb in size, a large GJB2 promoter, preferably about 1.0 kb in size, or a sequential combination of 2-3 individual GJB2 expression-specific promoters; operably linked to a 0.9 kb 3′-UTR regulatory region comprising the Woodchuck Hepatitis Virus Posttranscriptional Regulatory Element (WPRE) followed by either a SV40 or human growth hormone (hGH) polyadenylation signal, and further comprising either two about 143-base sequence-modulated inverted terminal repeats (ITRs) flanking the AAV genomic cassette or a self-complimentary AAV (scAAV) genomic cassette consisting of two inverted identical repeats, preferably no longer than 2.4 kb, separated by an about 113-base scAAV-enabling ITR (ITRΔtrs) and flanked on either end by about 143-base sequence-modulated ITRs.


SEQUENCE LISTING (41 sequences)

SEQ ID NO: 1 - DNA, 1680 bp, synthetic construct
  misc_feature 1..1680: note = Description of Artificial Sequence: Synthetic polynucleotide
  source 1..1680: mol_type = other DNA
ctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctaca

SEQ ID NO: 2 - DNA, 806 bp, synthetic construct
  misc_feature 1..806: note = Description of Artificial Sequence: Synthetic polynucleotide
  source 1..806: mol_type = other DNA
gagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacag

SEQ ID NO: 3 - DNA, 1055 bp, synthetic construct
  misc_feature 1..1055: note = Description of Artificial Sequence: Synthetic polynucleotide
  source 1..1055: mol_type = other DNA
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 4 - DNA, 961 bp, synthetic construct
  misc_feature 1..961: note = Description of Artificial Sequence: Synthetic polynucleotide
  source 1..961: mol_type = other DNA
cctcagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 5 - DNA, 1675 bp, synthetic construct
  misc_feature 1..1675: note = Description of Artificial Sequence: Synthetic polynucleotide
  source 1..1675: mol_type = other DNA
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 6 - DNA, 1000 bp, synthetic construct
  misc_feature 1..1000: note = Description of Artificial Sequence: Synthetic polynucleotide
  source 1..1000: mol_type = other DNA
ctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagct

SEQ ID NO: 7 - DNA, 143 bp, synthetic construct
  misc_feature 1..143: note = Description of Artificial Sequence: Synthetic polynucleotide
  source 1..143: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttc

SEQ ID NO: 8 - DNA, 143 bp, synthetic construct
  misc_feature 1..143: note = Description of Artificial Sequence: Synthetic polynucleotide
  source 1..143: mol_type = other DNA
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 9 - DNA, 106 bp, synthetic construct
  misc_feature 1..106: note = Description of Artificial Sequence: Synthetic polynucleotide
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 678 bp, synthetic construct
  misc_feature 1..678: note = Description of Artificial Sequence: Synthetic polynucleotide
  source 1..678: mol_type = other DNA
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 11 - DNA, 678 bp, synthetic construct
  misc_feature 1..678: note = Description of Artificial Sequence: Synthetic polynucleotide
  source 1..678: mol_type = other DNA
atggactggggcaccctgcagactatcctggggggcgtcaataagcattcaactagcatcggaaagatttggctgactgtcctgtttatctttcggatcatgatcctggtggtggcagcaaaggaagtgtggggcgacgagcaggccgatttcgtgtgcaacacactgcagccaggctgcaagaacgtgtgctacgaccactattttcccatctctcacatcaggctgtgggccctgcagctgatcttcgtgagcacccctgccctgctggtggcaatgcacgtggcctatcggagacacgagaagaagcgcaagtttatcaagggcgagatcaagagcgagttcaaggatatcgaggagatcaagacacagaaggtgaggatcgagggctccctgtggtggacctacacaagctccatcttctttcgcgtgatcttcgaggccgcctttatgtacgtgttctatgtgatgtacgacggcttttctatgcagcggctggtgaagtgcaacgcctggccctgtcctaatacagtggattgtttcgtgtccagacccaccgagaagacagtgttcaccgtgtttatgatcgccgtgtctggcatctgcatcctgctgaacgtgaccgagctgtgctatctgctgatccggtactgtagtggaaagagcaaaaaacccgtg

SEQ ID NO: 12 - DNA, 678 bp, synthetic construct
  misc_feature 1..678: note = Description of Artificial Sequence: Synthetic polynucleotide
  source 1..678: mol_type = other DNA
atggactggggaacattgcaaactattttgggaggagtcaacaagcattcaactagcatcgggaagatctggctgaccgtgctgttcatctttcgcatcatgattctcgtggtggccgctaaggaagtctggggcgatgaacaggccgacttcgtgtgtaacacgctgcagcccggttgcaaaaacgtctgctacgatcactacttccccatctcacacattagactgtgggcgctgcagctgattttcgtgtccaccccggcacttcttgtggcgatgcacgtggcctaccggcggcacgagaagaaaaggaagttcattaagggcgaaatcaagtccgagttcaaggacatcgaagaaatcaagacccagaaggtccgcattgagggctccctctggtggacctacacctcgtccatcttcttccgggtcatattcgaggccgcctttatgtacgtgttttacgtgatgtacgacggtttcagcatgcaaagactcgtcaagtgcaacgcttggccttgccccaataccgtggattgcttcgtgtcccgcccgaccgagaaaactgtgttcactgtgttcatgatcgccgtgtccggcatctgcatcctgctgaacgtgaccgagctgtgctatctcctgatccggtactgtagcggaaagtcgaagaagcctgtg

SEQ ID NO: 13 - DNA, 678 bp, synthetic construct
  misc_feature 1..678: note = Description of Artificial Sequence: Synthetic polynucleotide
  source 1..678: mol_type = other DNA
atggattgggggacgctccagactatacttggcggggtaaacaaacattccacctcaattggcaaaatctggctcacagtcctcttcatcttcagaataatgatactcgtggttgccgctaaagaagtttggggtgacgagcaagccgatttcgtctgtaacaccctccaaccaggttgcaaaaatgtctgttacgatcactactttcctattagccatattagactctgggccctgcaacttatcttcgtttccactcctgctctgctcgtcgctatgcacgttgcctatcgccgccatgaaaaaaaacggaaattcattaagggagagattaagagtgaattcaaggatattgaagagattaaaacgcaaaaagttagaattgagggatcactgtggtggacttataccagtagcatcttttttagggtcattttcgaagctgctttcatgtatgttttctatgtaatgtacgacggtttctccatgcaacgcttggttaaatgtaacgcctggccatgccctaatacggttgattgctttgtctcccgccctactgaaaagacagtgtttaccgttttcatgatcgccgtaagtggaatttgtatccttcttaacgtgaccgagttgtgctatctccttattcgctactgttcaggaaaaagtaaaaaaccagta

SEQ ID NO: 14 - DNA, 27 bp, synthetic construct
  misc_feature 1..27: note = Description of Artificial Sequence: Synthetic oligonucleotide
  source 1..27: mol_type = other DNA
tacccatacgatgttccagattacgct

SEQ ID NO: 15 - DNA, 589 bp, synthetic construct
  misc_feature 1..589: note = Description of Artificial Sequence: Synthetic polynucleotide
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 16 - DNA, 122 bp, synthetic construct
  misc_feature 1..122: note = Description of Artificial Sequence: Synthetic polynucleotide
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 17 - DNA, 208 bp, synthetic construct
  misc_feature 1..208: note = Description of Artificial Sequence: Synthetic polynucleotide
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 18 - DNA, 2208 bp, Adeno-associated dependoparvovirus A
  source 1..2208: mol_type = unassigned DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 19 - protein, 735 aa, Adeno-associated dependoparvovirus A [sequence omitted]

SEQ ID NO: 20 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttaagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaattctcgtggaccggtgctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtaccagattcctgactcgtaatctgtaa

SEQ ID NO: 21 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 22 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcaacgcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacagatggagaaaacaacaacagtgatttctcgtggactggagctaccaagtaccacctcaatggcagagactctctggtgaatccgggcccggccatggcaagccacaaggacgatgaagaaaagttttttcctcagagcggggttctcatctttgggaagcaaagcgcagcgggagcagatgtggcaattgatagtgtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 23 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKQSAAGADVAIDSVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcaacgcaggagccagcaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttgagcagaacaaacaccgcgagcggaaacgtcacgcagtcaacgcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacagggacgatgacgacaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 25 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTASGNVTQSTLQFSQAGASDIRDQSRNWLPGPCYRQQRVSKTSADNNNSEYSWTGATKYHLNGRDSLVNPGPAMASHRDDDDKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagagagtctcaaaaacagacggcgagaacaacaacagtgacttctcctggacaggagctaccaagtaccacctcaatggcagagactctctggtgaatcccggaccagctatggcaagccacaaggacgatgaagaaaagttttttcctcagagcggggttctcatctttgggaaggaagacgccaccgaaaacaatatcgacatcgaccgggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 27 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKEDATENNIDIDRVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - DNA, 2208 bp, synthetic construct; identical to SEQ ID NO: 18
  misc_feature 1..2208: note = Description of Artificial Sequence: Synthetic polynucleotide
  source 1..2208: mol_type = other DNA

SEQ ID NO: 29 - protein, 735 aa, synthetic construct [sequence omitted]

SEQ ID NO: 30 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcaacgcaggagccagcaacgacaatcacttctttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagatgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttaagcagaacaaacactccaagtggaaccaccacgcagtcaaggcttcagttttctcaggccggagcgagtgacattcgggaccagtctaggaactggcttcctggaccctgttaccgccagcagagagtctcaaaagtcgacggcgagaacaacaacagtgacttctcctggacaggagctaccaagtaccacctcaatggcagagactctctggtgaatccgggaccggccatggcaagccacaaggacgatgaagaaaagttttttcctcagagcggggttctcatctttgggaagcaaagcgccgccggagccgatgtcgcgatcgacagcgtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtaccagattcctgactcgtaatctgtaa

SEQ ID NO: 31 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKVDGENNNSDFSWTGATKYHLNGRDSLVNPGPAMASHKDDEEKFFPQSGVLIFGKQSAAGADVAIDSVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 32 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcatcaggagccagcaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagagagtctcaaaacaagacgccgagaacaacaacagtgagttctcctggccaggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaatagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 33 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKQDAENNNSEFSWPGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 34 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttgagcagaacaaacaccggggccggaaacatgacgacctcagcgcttaggttttctcaggccggagcgagtgacattcgggaccagtctaggaactggcttcctggaccctgttaccgccagcagcgagtatcaacaacacccgccgacaacaacaacagtgacttctcgtggactggagctaccaagtaccacctcaatggcagagactctctggtgaatccgggcccggccatggcaagccacaaggacgatgacgagaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 35 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTGAGNMTTSALRFSQAGASDIRDQSRNWLPGPCYRQQRVSTTPADNNNSDFSWTGATKYHLNGRDSLVNPGPAMASHKDDDEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 36 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagatgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagagagtctcaaaaacagacggcgagaacaacaacagtgacttctcctggacaggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggagatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 37 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - DNA, 2208 bp, synthetic construct
  misc_feature 1..2208: note = Description of Artificial Sequence: Synthetic polynucleotide
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcaacgcaggagccagcaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagatgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttaagcagaacaaacactccaagtggaaccaccacgcagtcaaggcttcagttttctcaggccggagcgagtgacattcgggaccagtctaggaactggcttcctggaccctgttaccgccagcagagagtctcaaaaacagacggcgagaacaacaacagtgacttctcctggacaggagctaccaagtaccacctcaatggcagagactctctggtgaatccgggaccggccatggcaagccacaaggacgatgaagaaaagttttttcctcagagcggggttctcatctttgggaagcaaagcgccgccggagccgatgtcgcgatcgacagcgtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtaccagattcctgactcgtaatctgtaa

SEQ ID NO: 39 - protein, 735 aa, synthetic construct
  REGION 1..735: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKQSAAGADVAIDSVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 40 - DNA, 24 bp, synthetic construct
  misc_feature 1..24: note = Description of Artificial Sequence: Synthetic oligonucleotide
  source 1..24: mol_type = other DNA
gactacaaagacgatgacgacaag

SEQ ID NO: 41 - protein, 8 aa, synthetic construct
  REGION 1..8: note = Description of Artificial Sequence: Synthetic peptide
DYKDDDDK